(19) United States
(12) Patent Application Publication
(10) Pub. No.: US 2022/0411782 A1
(43) Pub. Date: Dec. 29, 2022

(54) PHAGE COMPOSITIONS COMPRISING CRISPR-CAS SYSTEMS AND METHODS OF USE THEREOF

(71) Applicant: Locus Biosciences, Inc., Morrisville, NC (US)

(72) Inventors: David G. OUSTEROUT, Morrisville, NC (US); Kurt SELLE, Morrisville, NC (US)

(21) Appl. No.: 17/774,360

(22) PCT Filed: Nov. 5, 2020
(86) PCT No.: PCT/US2020/059218

§ 371 Date: May 4, 2022

Related U.S. Application Data
(60) Provisional application No. 62/931,797, filed on Nov. 6, 2019.

(51) Int. Cl. C12N 15/10; A61K 31/713; A61K 35/74; A61K 35/76; C12N 15/86; C12N 7/00

(52) U.S. Cl. CPC C12N 15/102; A61K 31/713; A61K 35/74; A61K 35/76; C12N 15/86; C12N 7/00; C12N 2310/20; C12N 2795/00041; C12N 2795/10043

(57) ABSTRACT

Disclosed here are phage compositions comprising Type I CRISPR-Cas systems and methods of use thereof. In some embodiments, disclosed herein is a nucleic acid sequence comprising (a) a first CRISPR array designed to be operable with a first Type I CRISPR-Cas system, and (b) a second CRISPR array designed to be operable with a second Type I CRISPR-Cas system, wherein the first Type I CRISPR-Cas system and the second Type I CRISPR-Cas system are different Type I CRISPR-Cas systems.

### CROSS-REFERENCE

This application claim the benefit of U.S. Patent Application No. 62/931,797, filed Nov. 6, 2019, which is hereby incorporated by reference in its entirety.

### SUMMARY

Disclosed herein, in certain embodiments, are phage compositions comprising Type I CRISPR-Cas systems and methods of use thereof.

In some embodiments, disclosed herein is a nucleic acid sequence comprising (a) a first CRISPR array designed to be operable with a first Type I CRISPR-Cas system, and (b) a second CRISPR array designed to be operable with a second Type I CRISPR-Cas system, wherein the first Type I CRISPR-Cas system and the second Type I CRISPR-Cas system are different Type I CRISPR-Cas systems. In some embodiments, the first CRISPR array comprises a first spacer sequence and the second CRISPR array comprises a second spacer sequence. In some embodiments, the first CRISPR array and the second CRISPR array further comprises at least one repeat sequence. In some embodiments, the at least one repeat sequence is operably linked to the first spacer sequence at either its 5′ end or its 3′ end and/or the second spacer sequence at either its 5′ end or its 3′ end. In some embodiments, the first and/or second spacer sequence is complementary to a target nucleotide sequence of an essential gene in a target bacterium. In some embodiments, the target nucleotide sequence comprises all or a part of a promoter sequence of the essential gene. In some embodiments, the target nucleotide sequence comprises all or a part of a nucleotide sequence located on a coding strand of a transcribed region of the essential gene. In some embodiments, the essential gene is ftsA. In some embodiments, the first and/or second spacer sequence is complementary to a target nucleotide sequence in a non-essential gene. In some embodiments, the first and/or second spacer is completely to a target nucleic acid sequence in a noncoding sequence. In some embodiments, the first CRISPR array and the second CRISPR array are on same nucleic acid sequence. In some embodiments, the nucleic acid sequence further comprises a leuO coding sequence. In some embodiments, the nucleic acid sequence further comprises a leader sequence. In some embodiments, the nucleic acid sequence further comprises a promoter sequence. In some embodiments, the nucleic acid sequence is at least 80%, at least 90%, at least 95%, at least 99%, or 100% identical to a nucleic acid sequence set forth as SEQ ID NO: 1. In some embodiments, the first Type I CRISPR-Cas system is a Type I-A system, Type I-B system, Type I-C system, Type I-D system, Type I-E system, or Type I-F system. In some embodiments, the first Type I CRISPR-Cas system is a Type I-E system. In some embodiments, the second Type I CRISPR-Cas system is a Type I-A system, Type I-B system, Type I-C system, Type I-D system, Type I-E system, or Type I-F system. In some embodiments, the second Type I CRISPR-Cas system is a Type I-F system. In some embodiments, the first Type I CRISPR-Cas system, the second Type I CRISPR-Cas system, or both are endogenous to the target bacterium. In some embodiments, the first Type I CRISPR-Cas system, the second Type I CRISPR-Cas system, or both are exogenous to the target bacterium. In some embodiments, the target bacterium is E. coli. In some embodiments, the E. coli is a multidrug-resistant (MDR) strain. In some embodiments, the E. coli is an extended spectrum beta-lactamase (ESBL) strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the E. coli causes urinary tract infection. In some embodiments, the E. coli causes inflammatory bowel disease (IBD). A bacteriophage comprising a nucleic acid sequence comprising (a) a first CRISPR array designed to be operable with a first Type I CRISPR-Cas system, and (b) a second CRISPR array designed to be operable with a second Type I CRISPR-Cas system, wherein the first Type I CRISPR-Cas system and the second Type I CRISPR-Cas system are different Type I CRISPR-Cas systems. In some embodiments, the first CRISPR array comprises a first spacer sequence and the second CRISPR array comprises a second spacer sequence. In some embodiments, the first CRISPR array and the second CRISPR array further comprises at least one repeat sequence. In some embodiments, the at least one repeat sequence is operably linked to the first spacer sequence at either its 5′ end or its 3′ end and/or the second spacer sequence at either its 5′ end or its 3′ end. In some embodiments, the first spacer sequence and/or the second spacer sequence is complementary to a target nucleotide sequence of an essential gene in a target bacterium. In some embodiments, the target nucleotide sequence comprises all or a part of a promoter sequence of the essential gene. In some embodiments, the target nucleotide sequence comprises all or a part of a nucleotide sequence located on a coding strand of a transcribed region of the essential gene. In some embodiments, the essential gene is ftsA. In some embodiments, the first CRISPR array and the second CRISPR array are on same nucleic acid sequence. In some embodiments, the nucleic acid sequence further comprises a leuO coding sequence. In some embodiments, the nucleic acid sequence further comprises a leader sequence. In some embodiments, the nucleic acid sequence further comprises a promoter sequence. In some embodiments, the nucleic acid sequence is at least 80%, at least 90%, at least 95%, at least 99%, or 100% identical to a nucleic acid sequence set forth as SEQ ID NO: 1. In some embodiments, the first Type I CRISPR-Cas system is a Type I-A system, Type I-B system, Type I-C system, Type I-D system, Type I-E system, or Type I-F system. In some embodiments, the first Type I CRISPR-Cas system is a Type I-E system. In some embodiments, the second Type I CRISPR-Cas system is a Type I-A system, Type I-B system, Type I-C system, Type I-D system, Type I-E system, or Type I-F system. In some embodiments, the second Type I CRISPR-Cas system is a Type I-F system. In some embodiments, the first Type I CRISPR-Cas system, the second Type I CRISPR-Cas system, or both are endogenous to the target bacterium. In some embodiments, the first Type I CRISPR-Cas system, the second Type I CRISPR-Cas system, or both are exogenous to the target bacterium. In some embodiments, the target bacterium is killed solely by lytic activity of the bacteriophage. In some embodiments, the target bacterium is killed solely by activity of the first Type I CRISPR-Cas system or the second Type I CRISPR-Cas system. In some embodiments, the target bacterium is killed by lytic activity of the bacteriophage in combination with activity of the first Type I CRISPR-Cas system or the second Type I CRISPR-Cas system. In some embodiments, the target bacterium is killed by the activity of the first Type I CRISPR-Cas system or the second Type I CRISPR-Cas system, independently of the lytic activity of the bacteriophage. In some embodiments, the activity of the first Type I CRISPR-Cas system or the second Type I CRISPR-Cas system supplements or enhances the lytic activity of the bacteriophage. In some embodiments, the lytic activity of the bacteriophage and the activity of the first Type I CRISPR-Cas system or the second Type I CRISPR-Cas system are synergistic. In some embodiments, the lytic activity of the bacteriophage, the activity of the first Type I CRISPR-Cas system or the second Type I CRISPR-Cas system, or both, is modulated by a concentration of the bacteriophage. In some embodiments, the target bacterium is E. coli. In some embodiments, the E. coli is a multidrug-resistant (MDR) strain. In some embodiments, the E. coli is an extended spectrum beta-lactamase (ESBL) strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the E. coli causes urinary tract infection. In some embodiments, the E. coli causes inflammatory bowel disease (IBD). In some embodiments, the bacteriophage is an obligate lytic bacteriophage. In some embodiments, the bacteriophage is a temperate bacteriophage with a lysogeny gene removed, replaced, or inactivated, thereby rendering the bacteriophage lytic. In some embodiments, the bacteriophage is PTA-126317, PTA-126320, PTA-126316, PTA-126324, PTA-126315, or PTA-126319. In some embodiments, the nucleic acid sequence is inserted into a non-essential bacteriophage gene. In some embodiments, the bacteriophage is at least 80%, at least 90%, at least 95%, at least 99%, or 100% identical to a bacteriophage deposited under ATCC Patent Deposit number PTA-126317. In some embodiments, the bacteriophage is at least 80%, at least 90%, at least 95%, at least 99%, or 100% identical to a bacteriophage deposited under ATCC Patent Deposit number PTA-126316. In some embodiments, the bacteriophage is at least 80%, at least 90%, at least 95%, at least 99%, or 100% identical to a bacteriophage deposited under ATCC Patent Deposit number PTA-126324. In some embodiments, the bacteriophage is at least 80%, at least 90%, at least 95%, at least 99%, or 100% identical to a bacteriophage deposited under ATCC Patent Deposit number PTA-126315. In some embodiments, the bacteriophage is at least 80%, at least 90%, at least 95%, at least 99%, or 100% identical to a bacteriophage deposited under ATCC Patent Deposit number PTA-126319.

In some embodiments, disclosed herein is a PTA-126317 bacteriophage comprising a nucleic acid sequence comprising (a) a first CRISPR array designed to be operable with a first Type I CRISPR-Cas system, or (b) a leuO coding sequence. In some embodiments, disclosed herein is a PTA-126320 bacteriophage comprising a nucleic acid sequence comprising (a) a first CRISPR array designed to be operable with a first Type I CRISPR-Cas system, or (b) a leuO coding sequence. In some embodiments, disclosed herein is a PTA-126316 bacteriophage comprising a nucleic acid sequence comprising (a) a first CRISPR array designed to be operable with a first Type I CRISPR-Cas system, or (b) a leuO coding sequence. In some embodiments, disclosed herein is a PTA-126324 bacteriophage comprising a nucleic acid sequence comprising (a) a first CRISPR array designed to be operable with a first Type I CRISPR-Cas system, or (b) a leuO coding sequence. In some embodiments, disclosed herein is a PTA-126315 bacteriophage comprising a nucleic acid sequence comprising (a) a first CRISPR array designed to be operable with a first Type I CRISPR-Cas system, or (b) a leuO coding sequence. In some embodiments, disclosed herein is a PTA-126319 bacteriophage comprising a nucleic acid sequence comprising (a) a first CRISPR array designed to be operable with a first Type I CRISPR-Cas system, or (b) a leuO coding sequence. In some embodiments, the nucleic acid sequence comprises (a) a first CRISPR array designed to be operable with a first Type I CRISPR-Cas system. In some embodiments, the nucleic acid sequence comprises (b) a leuO coding sequence. In some embodiments, the nucleic acid sequence comprises (a) a first CRISPR array designed to be operable with a first Type I CRISPR-Cas system, and (b) a leuO coding sequence. In some embodiments, the nucleic acid sequence further comprises (c) a second CRISPR array designed to be operable with a second Type I CRISPR-Cas system. In some embodiments, the first Type I CRISPR-Cas system and the second Type I CRISPR-Cas system are different Type I CRISPR-Cas systems. In some embodiments, the first CRISPR array comprises first spacer sequence and the second CRISPR array comprises a second spacer sequence. In some embodiments, the first CRISPR array and the second CRISPR array further comprises at least one repeat sequence. In some embodiments, the at least one repeat sequence is operably linked to the first spacer sequence at either its 5′ end or its 3′ end and/or the second spacer sequence at either its 5′ end or its 3′ end. In some embodiments, the first spacer sequence and/or the second spacer sequence is complementary to a target nucleotide sequence of an essential gene in a target bacterium. In some embodiments, the target nucleotide sequence comprises all or a part of a promoter sequence of the essential gene. In some embodiments, the target nucleotide sequence comprises all or a part of a nucleotide sequence located on a coding strand of a transcribed region of the essential gene. In some embodiments, the essential gene is ftsA. In some embodiments, the first CRISPR array and the second CRISPR array are on same nucleic acid sequence. In some embodiments, the nucleic acid sequence further comprises a leader sequence. In some embodiments, the nucleic acid sequence further comprises a promoter sequence. In some embodiments, the nucleic acid sequence is at least 80%, at least 90%, at least 95%, at least 99%, or 100% identical to a nucleic acid sequence set forth as SEQ ID NO: 1. In some embodiments, the first Type I CRISPR-Cas system is a Type I-A system, Type I-B system, Type I-C system, Type I-D system, Type I-E system, or Type I-F system. In some embodiments, the first Type I CRISPR-Cas system is a Type I-E system. In some embodiments, the second Type I CRISPR-Cas system is a Type I-A system, Type I-B system, Type I-C system, Type I-D system, Type I-E system, or Type I-F system. In some embodiments, the second Type I CRISPR-Cas system is a Type I-F system. In some embodiments, the first Type I CRISPR-Cas system, the second Type I CRISPR-Cas system, or both are endogenous to the target bacterium. In some embodiments, the first Type I CRISPR-Cas system, the second Type I CRISPR-Cas system, or both are exogenous to the target bacterium. In some embodiments, the target bacterium is killed solely by lytic activity of the bacteriophage. In some embodiments, the target bacterium is killed solely by activity of the first Type I CRISPR-Cas system or the second Type I CRISPR-Cas system. In some embodiments, the target bacterium is killed by lytic activity of the bacteriophage in combination with activity of the first Type I CRISPR-Cas system or the second Type I CRISPR-Cas system. In some embodiments, the target bacterium is killed by the activity of the first Type I CRISPR-Cas system or the second Type I CRISPR-Cas system, independently of the lytic activity of the bacteriophage. In some embodiments, the activity of the first Type I CRISPR-Cas system or the second Type I CRISPR-Cas system supplements or enhances the lytic activity of the bacteriophage. In some embodiments, the lytic activity of the bacteriophage and the activity of the first Type I CRISPR-Cas system or the second Type I CRISPR-Cas system are synergistic. In some embodiments, the lytic activity of the bacteriophage, the activity of the first Type I CRISPR-Cas system or the second Type I CRISPR-Cas system, or both, is modulated by a concentration of the bacteriophage. In some embodiments, the target bacterium is E. coli. In some embodiments, the E. coli is a multidrug-resistant (MDR) strain. In some embodiments, the E. coli is an extended spectrum beta-lactamase (ESBL) strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the E. coli causes urinary tract infection. In some embodiments, the E. coli causes inflammatory bowel disease (IBD). In some embodiments, the bacteriophage is an obligate lytic bacteriophage. In some embodiments, the nucleic acid sequence is inserted into a non-essential bacteriophage gene. In some embodiments, the bacteriophage is at least 80%, at least 90%, at least 95%, at least 99%, or 100% identical to a bacteriophage deposited under ATCC Patent Deposit number PTA-126317. In some embodiments, the bacteriophage is at least 80%, at least 90%, at least 95%, at least 99%, or 100% identical to a bacteriophage deposited under ATCC Patent Deposit number PTA-126320. In some embodiments, the bacteriophage is at least 80%, at least 90%, at least 95%, at least 99%, or 100% identical to a bacteriophage deposited under ATCC Patent Deposit number PTA-126316. In some embodiments, the bacteriophage is at least 80%, at least 90%, at least 95%, at least 99%, or 100% identical to a bacteriophage deposited under ATCC Patent Deposit number PTA-126324. In some embodiments, the bacteriophage is at least 80%, at least 90%, at least 95%, at least 99%, or 100% identical to a bacteriophage deposited under ATCC Patent Deposit number PTA-126315. In some embodiments, the bacteriophage is at least 80%, at least 90%, at least 95%, at least 99%, or 100% identical to a bacteriophage deposited under ATCC Patent Deposit number PTA-126319.

In some embodiments, disclosed herein is a composition comprising: at least two bacteriophages selected from a list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126316, (iv) a bacteriophage comprising at least 80% identity to a bacteriophage deposited under ATCC Patent Deposit number PTA-126324, (v) a bacteriophage comprising at least 80% identity to a bacteriophage deposited under ATCC Patent Deposit number PTA-126315, and (vi) a bacteriophage comprising at least 80% identity to a bacteriophage deposited under ATCC Patent Deposit number PTA-126319.

In some embodiments, disclosed herein is a composition comprising: at least three bacteriophages selected from a list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126316, (iv) a bacteriophage comprising at least 80% identity to a bacteriophage deposited under ATCC Patent Deposit number PTA-126324, (v) a bacteriophage comprising at least 80% identity to a bacteriophage deposited under ATCC Patent Deposit number PTA-126315, and (vi) a bacteriophage comprising at least 80% identity to a bacteriophage deposited under ATCC Patent Deposit number PTA-126319.

In some embodiments, disclosed herein is a composition comprising: at least six bacteriophages selected from a list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126316, (iv) a bacteriophage comprising at least 80% identity to a bacteriophage deposited under ATCC Patent Deposit number PTA-126324, (v) a bacteriophage comprising at least 80% identity to a bacteriophage deposited under ATCC Patent Deposit number PTA-126315, and (vi) a bacteriophage comprising at least 80% identity to a bacteriophage deposited under ATCC Patent Deposit number PTA-126319.

In some embodiments, disclosed herein is a composition comprising: (a) a bacteriophage comprising at least 80% identity to a bacteriophage deposited under ATCC Patent Deposit number PTA-126324; (b) a bacteriophage comprising at least 80% identity to a bacteriophage deposited under ATCC Patent Deposit number PTA-126315; and (c) a bacteriophage comprising at least 80% identity to a bacteriophage deposited under ATCC Patent Deposit number PTA-126319. In some embodiments, (a) the bacteriophage comprises at least 90%, at least 95%, at least 99%, or 100% identity to a bacteriophage deposited under ATCC Patent Deposit number PTA-126324. In some embodiments, (b) the bacteriophage comprises at least 90%, at least 95%, at least 99%, or 100% identity to a bacteriophage deposited under ATCC Patent Deposit number PTA-126315. In some embodiments, (c) the bacteriophage comprises at least 90%, at least 95%, at least 99%, or 100% identity to a bacteriophage deposited under ATCC Patent Deposit number PTA-126319.

In some embodiments, disclosed herein is a composition comprising: (a) a bacteriophage comprising at least 80% identity to a bacteriophage deposited under ATCC Patent Deposit number PTA-126317, and (b) at least one more bacteriophage selected from the list consisting of: (i) a bacteriophage comprising at least 80% identity to a bacteriophage deposited under ATCC Patent Deposit number PTA-126320, (ii) a bacteriophage comprising at least 80% identity to a bacteriophage deposited under ATCC Patent Deposit number PTA-126316, (iii) a bacteriophage comprising at least 80% identity to a bacteriophage deposited under ATCC Patent Deposit number PTA-126324, (iv) a bacteriophage comprising at least 80% identity to a bacteriophage deposited under ATCC Patent Deposit number PTA-126315, and (v) a bacteriophage comprising at least 80% identity to a bacteriophage deposited under ATCC Patent Deposit number PTA-126319.

In some embodiments, disclosed herein is a composition comprising: (a) a bacteriophage comprising at least 80% identity to a bacteriophage deposited under ATCC Patent Deposit number PTA-126320, and (b) at least one more bacteriophage selected from the list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126316, (iii) a bacteriophage comprising at least 80% identity to a bacteriophage deposited under ATCC Patent Deposit number PTA-126324, (iv) a bacteriophage comprising at least 80% identity to a bacteriophage deposited under ATCC Patent Deposit number PTA-126315, and (v) a bacteriophage comprising at least 80% identity to a bacteriophage deposited under ATCC Patent Deposit number PTA-126319.

In some embodiments, disclosed herein is a composition comprising: (a) a bacteriophage comprising at least 80% identity to a bacteriophage deposited under ATCC Patent Deposit number PTA-126316, and (b) at least one more bacteriophage selected from the list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126324, (iv) a bacteriophage comprising at least 80% identity to a bacteriophage deposited under ATCC Patent Deposit number PTA-126315, and (v) a bacteriophage comprising at least 80% identity to a bacteriophage deposited under ATCC Patent Deposit number PTA-126319.

In some embodiments, disclosed herein is a composition comprising: (a) a bacteriophage comprising at least 80% identity to a bacteriophage deposited under ATCC Patent Deposit number PTA-126324, and (b) at least one more bacteriophage selected from the list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126316, (iv) a bacteriophage comprising at least 80% identity to a bacteriophage deposited under ATCC Patent Deposit number PTA-126315, and (v) a bacteriophage comprising at least 80% identity to a bacteriophage deposited under ATCC Patent Deposit number PTA-126319.

In some embodiments, disclosed herein is a composition comprising: (a) a bacteriophage comprising at least 80% identity to a bacteriophage deposited under ATCC Patent Deposit number PTA-126315, and (b) at least one more bacteriophage selected from the list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126316, (iv) a bacteriophage comprising at least 80% identity to a bacteriophage deposited under ATCC Patent Deposit number PTA-126324, (v) a bacteriophage comprising at least 80% identity to a bacteriophage deposited under ATCC Patent Deposit number PTA-126319.

In some embodiments, disclosed herein is a composition comprising: (a) a bacteriophage comprising at least 80% identity to a bacteriophage deposited under ATCC Patent Deposit number PTA-126319, and (b) at least one more bacteriophage selected from the list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126316, (iv) a bacteriophage comprising at least 80% identity to a bacteriophage deposited under ATCC Patent Deposit number PTA-126324, and (v) a bacteriophage comprising at least 80% identity to a bacteriophage deposited under ATCC Patent Deposit number PTA-126315. In some embodiments, disclosed herein is a pharmaceutical composition comprising: (a) (i) the nucleic acid sequence disclosed herein) the bacteriophage disclosed herein, or (iii) the composition disclosed herein; and (b) a pharmaceutically acceptable excipient. In some embodiments, the pharmaceutical composition is in the form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof. In some embodiments, disclosed herein is a method of killing a target bacterium comprising introducing into a target bacterium (a) the bacteriophage disclosed herein, (b) the composition disclosed herein, or (c) the pharmaceutical composition disclosed herein. In some embodiments, disclosed herein is a method modifying a mixed population of bacterial cells having a first bacterial species that comprises a target nucleotide sequence in the essential gene and a second bacterial species that does not comprise a target nucleotide sequence in the essential gene, the method comprising introducing into the mixed population of bacterial cells (a) the bacteriophage disclosed herein, (b) the composition disclosed herein, or (c) the pharmaceutical composition disclosed herein. In some embodiments, disclosed herein is a method of treating a disease in an individual in need thereof, the method comprising administering to the individual (a) the bacteriophage disclosed herein, (b) the composition disclosed herein, or (c) the pharmaceutical composition disclosed herein. In some embodiments, the disease is a bacterial infection. In some embodiments, the disease is a urinary tract infection (UTI). In some embodiments, the disease is inflammatory bowel disease (IBD). In some embodiments, the individual is a mammal. In some embodiments, the administering is intra-arterial, intravenous, intraurethral, intramuscular, oral, subcutaneous, inhalation, or any combination thereof. In some embodiments, (a) the bacteriophage, (b) the composition, or (c) the pharmaceutical composition is administered at a dose of phage between 10⁶ and 10¹⁰ PFU. In some embodiments, (a) the bacteriophage, (b) the composition, or (c) the pharmaceutical composition is administered 1, 2, 3, 4, or 5 times daily. In some embodiments, (a) the bacteriophage, (b) the composition, or (c) the pharmaceutical composition is administered 2 times daily. In some embodiments, (a) the bacteriophage, (b) the composition, or (c) the pharmaceutical composition is administered every 12 hours. In some embodiments, disclosed herein is a method of treating a urinary tract infection (UTI) in an individual in need thereof, the method comprising administering to the individual (a) the bacteriophage disclosed herein, (b) the composition disclosed herein, or (c) the pharmaceutical composition disclosed herein. In some embodiments, the UTI is caused by E. coli. In some embodiments, the E. coli is a multidrug-resistant (MDR) strain. In some embodiments, the E. coli is an extended spectrum beta-lactamase (ESBL) strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the individual is a mammal. In some embodiments, the administering is intra-arterial, intravenous, intraurethral, intramuscular, oral, subcutaneous, inhalation, or any combination thereof. In some embodiments, (a) the bacteriophage, (b) the composition, or (c) the pharmaceutical composition is administered at a dose of phage between 10⁶ and 10¹⁰ PFU. In some embodiments, (a) the bacteriophage, (b) the composition, or (c) the pharmaceutical composition is administered 1, 2, 3, 4, or 5 times daily. In some embodiments, (a) the bacteriophage, (b) the composition, or (c) the pharmaceutical composition is administered 2 times daily. In some embodiments, (a) the bacteriophage, (b) the composition, or (c) the pharmaceutical composition is administered every 12 hours.

### BRIEF DESCRIPTION OF THE DRAWINGS

The novel features of the disclosures are set forth with particularity in the appended claims. A better understanding of the features and advantages of the present disclosures will be obtained by reference to the following detailed description that sets forth illustrative embodiments, in which the principles of the disclosures are utilized, and the accompanying drawings of which:

FIG. 1 illustrates the crRNA array and leuO expression cassettes used to determine crRNA strain coverage and activity.

FIG. 2A-FIG. 2C exemplify E. coli strain panel and crRNA coverage and activity. FIG. 2A exemplifies pathotype distribution of E. coli genomes analyzed. UPEC-uropathogenic E. coli, STEC-shiga toxin E. coli, DEC-diarrheagenic E. coli, EPEC-enteropathogenic E. coli. FIG. 2B exemplifies strain coverages of CRISPR machinery based on genomic query for the ftsA gene target and both Type I-E and Type I-F CRISPR-Cas systems. FIG. 2C exemplifies functional assessment of lethality by the combined Type I-E and Type I-F ftsA-targeting crRNAs complexed with the leuO expression cassette. A plasmid expressing each individual crRNA was transformed into recipient E. coli strains containing Type I-E, Type I-F, or no CRISPR-Cas3 systems, respectively.

FIG. 3 exemplifies M13-derived phagemid delivery of CRISPR constructs designed to test dependence of CRISPR-mediated lethality on leuO expression. Indicated E. coli strains were infected with 10⁹ transducing units/mL of each M13 phagemid and plated on selective media to recover transduced cells and count surviving colony forming units (transductants). The X-axis denotes E. coli strains tested: EMG2 (wild-type K12 strain), ΔHns (K12 strain lacking H-NS repression), BW25113 (engineered strain constitutively expressing a Type I-E CRISPR-Cas operon), and BW25113ΔCRISPR (engineered strain with deletion of entire Type I-E CRISPR-Cas operon). Each strain was transduced with the following phagemids indicated in the legend: Control, generic M13 transduction control; pCRISPR, phagemid that constitutively expresses non-targeting crRNA; leuO, phagemid that constitutively expresses the E. coli leuO gene; ftsA, phagemid that constitutively expresses crRNA targeting conserved ftsA gene present in E. coli; and ftsA::leuO, phagemid that constitutively expresses leuO gene and crRNA targeting ftsA.

FIG. 4A-FIG. 4C exemplify colony forming unit counts after treatment with wild-type phage (wt) or CRISPR-enhanced phage (cr) at 2 hours for crT7M (FIG. 4A), 5 hours for crT4 (FIG. 4B), and 2 hours for crT7 (FIG. 4C). The crT7M data are from a single experiment, the data from crT4 are from 3 independent experiments, and the data from crT7 are from 2 independent experiments. The x-axis denotes each phage and the MOI.

FIG. 5 exemplifies dose-response of host range percentage and durability percentage with the crPhage cocktail in MOI ranges from 10⁻⁷ to 10

FIG. 6A-FIG. 6D exemplify concentration of E. coli (CFU/g) in the bladders and kidneys of infected mice at 30 (6 h after the first dose by IV or IU) hours after infection (FIG. 6A-FIG. 6B) and 78 (6 hours after the fifth dose) hours after infection (FIG. 6C-FIG. 6D). Vertical bars represent the standard deviation.

FIG. 7A-FIG. 7C exemplify concentration of E. coli (CFU/g) in the bladder, spleen, and kidneys of infected mice at 54 (6 h after the first dose by IV) hours after infection. FIG. 7D-FIG. 7F exemplify concentration of E. coli (CFU/g) in the bladder, spleen, and kidneys of infected mice at 54 (6 h after the first dose by IU) hours after infection. FIG. 7G-FIG. 7I exemplify concentration of E. coli (CFU/g) in the bladder, spleen, and kidneys of infected mice at 54 (6 h after the first dose by IV+IU) hours after infection. FIG. 7J-FIG. 7L exemplify concentration of E. coli (CFU/g) in the bladder, spleen, and kidneys of infected mice at 102 (6 h after the fifth dose by IV) hours after infection. FIG. 7M-FIG. 7O exemplify concentration of E. coli (CFU/g) in the bladder, spleen, and kidneys of infected mice at 102 (6 h after the fifth dose by IU) hours after infection. FIG. 7P-FIG. 7R exemplify concentration of E. coli (CFU/g) in the bladder, spleen, and kidneys of infected mice at 102 (6 h after the fifth dose by IV+IU) hours after infection. Vertical bars represent the standard deviation.

FIG. 8A-FIG. 8B exemplify concentration of E. coli (CFU/g) in the bladder and kidneys of infected mice at 54 (6 h after the first dose by IV) hours after infection. FIG. 8C-FIG. 8D exemplify concentration of E. coli (CFU/g) in the bladder and kidneys of infected mice at 54 (6 h after the first dose by IU) hours after infection. FIG. 8E-FIG. 8F exemplify concentration of E. coli (CFU/g) in the bladder and kidneys of infected mice at 102 (6 h after the fifth dose by IV) hours after infection. FIG. 8G-FIG. 8H exemplify concentration of E. coli (CFU/g) in the bladder and kidneys of infected mice at 102 (6 h after the fifth dose by IU) hours after infection.

FIG. 9A-FIG. 9B exemplify concentration of E. coli (CFU/g) in the bladder and kidneys of infected mice at 54 (6 h after the first dose by IV) hours after infection. FIG. 9C-FIG. 9D exemplify concentration of E. coli (CFU/g) in the bladder and kidneys of infected mice at 54 (6 h after the first dose by IU) hours after infection. FIG. 9E-FIG. 9F exemplify concentration of E. coli (CFU/g) in the bladder and kidneys of infected mice at 102 (6 h after the fifth dose by IV) hours after infection. FIG. 9G-FIG. 9H exemplify concentration of E. coli (CFU/g) in the bladder and kidneys of infected mice at 102 (6 h after the fifth dose by IU) hours after infection.

FIG. 10A-FIG. 10B exemplify CFUs following IU in the bladder of mice. FIG. 10C-FIG. 10D exemplify CFUs following IU Administration in the kidney of mice. CFU=colony forming unit; Cipro=ciprofloxacin; crPhage=engineered bacteriophages; LR=Lactated Ringer's; IU=intraurethral; IV=intravenous.

FIG. 11A-FIG. 11B exemplify PFUs following IU Administration in the bladder of mice. FIG. 11C-FIG. 11D exemplify PFUs following IU Administration in the kidney of mice. FIG. 11E-FIG. 11F exemplify PFUs following IU Administration in the blood of mice. FIG. 11G exemplify PFUs following IU Administration in the urine of mice. crPhage=engineered bacteriophages; PFU=plaque forming unit; IU=intraurethral.

FIG. 12 exemplifies change in body weight (g) of mice treated with saline, crT7M, crT4, crT7 or crPhage Cocktail.

FIG. 13 exemplifies mean body temperatures (° C.) of mice treated with saline, crT7M, crT4, crT7 or crPhage Cocktail.

FIG. 14A-FIG. 14C exemplify treatment outline for tolerability (FIG. 14A), peritonitis model (FIG. 14B), and Thigh model (FIG. 14C).

FIG. 15A-FIG. 15F exemplify tolerability studies after IP administrations. Tolerability studies after IP administration of 2.0×10¹¹ PFU/mouse of crT7 (FIG. 15A-FIG. 15B), 3.7×10⁹ PFU/mouse of crT7M (FIG. 15C-FIG. 15D) or 6.0×10⁸ PFU/mouse of crT4 (FIG. 15E-FIG. 15F). Control animals were treated with saline injections only. Controls are indicated with a black closed circle, test condition is gray.

FIG. 16A-FIG. 16C exemplify protection in a lethal challenge peritonitis model of E. coli. Animals were injected IP with 5×10⁷ CFU/mouse of ATCC 8739 (fecal isolate) in 5% mucin (FIG. 16A-FIG. 16B) or 6×10⁷ CFU/mouse MG1655 in 2% mucin (FIG. 16C). Thirty minutes later, this was followed by a single IP dose 2.0×10¹¹ PFU/mouse of crT7 (FIG. 16A), 3.7×10⁹ PFU/mouse of crT7M (FIG. 16B) or 6.0×10⁸ PFU/mouse of crT4 (FIG. 16C). Control animals treated with saline injections only.

FIG. 17A-FIG. 17D exemplify bioburden reduction in a mouse thigh model. E. coli strain MG1655 was injected directly into the thigh muscle of mice 30 minutes prior to intramuscular injection with the indicated crPhage or 1× tris-buffered saline (phage vehicle). Each mouse (N=3 per condition/time point) received approximately 2.0×10¹¹ PFU/dose of crT7 (FIG. 17A), 2.0×10¹⁰ PFU/dose of crT4 (FIG. 17B), 4.0×10¹¹ PFU/dose of crT7M (FIG. 17C), or a cocktail (‘Cocktail’) containing 1.0×10¹⁰ PFU/dose of each crT7, crT7M and crT4 (FIG. 17D). Whole thigh muscles were excised at the indicated time points, homogenized and immediately diluted and plated to count surviving bacterial colonies per gram of tissue.

FIG. 18 exemplifies phage titration detection of crPhages in the murine urinary tract and other organs after single IU dose (Study 15(a)). A single-dose of a solution containing approximately 1.0×10⁹ PFU of each crPhage was administered by intraurethral instillation to N=3 mice per condition/time point. Animals were sacrificed at various time points and whole tissue homogenates were diluted and subjected to phage titration analysis to quantify the total combined amount of crT7 and crT7M. Means±SEM shown are the result of 3 technical replicates from 3 animals and quantify PFUs per gram (bladder, kidney, liver, or spleen) or per milliliter (blood) of either crT7 or crT7M (assay able to detect either phage). IU=intraurethral; PFU=plaque forming unit; SEM=standard error of the mean.

FIG. 19 exemplifies quantitative PCR detection of crPhages in the murine GI tract and other organs after single oral dose (Study 15(b)). A single-dose of a solution containing 2.7×10⁹ PFU of each phage was administered by oral gavage to N=3 mice per condition/time point. Animals were sacrificed at various time points and total DNA from whole tissue homogenates was extracted and subjected to qPCR analysis to quantify the amount of crT7 (A), crT4 (B) or crT7M (C) present. Means±SEM shown are the result of 3 technical replicates from 3 animals. The dotted line on each plot shows the limit of linearity for the assay; values below this line are shown for completeness, but are outside of the linear dynamic range of the assay. Values with an asterisk indicate the no signal was detectable. DNA=deoxyribonucleic acid; GI=gastrointestinal; PCR=polymerase chain reaction; PFU=plaque forming unit; SEM=standard error of the mean.

FIG. 20A-FIG. 20E exemplify phage dose-responses in E. coli bacterial growth measured by OD 630 nm readings. E. coli strain MG1655 was grown to mid-log phase and treated with MOI as follows: crT7 was incubated at MOIs of 0.0001 (thick dashed line), 0.01 (dotted line), and 1.0 (dashed line) (FIG. 20A). crT7M was incubated at MOIs of 0.0009 (thick line), 0.09 (dotted line), and 9.0 (dashed line) (FIG. 20B). crT4 was incubated at MOIs of 0.0006 (thick dashed line), 0.06 (dotted line), and 6.0 (dashed line) (FIG. 20C). Each phage was mixed in equal amounts to create a crPhage cocktail (‘Cocktail’) and was incubated at MOIs (for each crPhage) of 0.0006 (thick dashed line), 0.06 (dotted line), and 6.0 (dashed line) (FIG. 20D). FIG. 20E shows the first 2 hours from graph (FIG. 20D).

FIG. 21 exemplifies dose-responses measured as time-to-lysis. E. coli strain MG1655 was grown to mid-log phase and treated with MOI as indicated for each crPhage. Growth curves were fitted to the curve and the first derivatives of the smoothed lines were determined using the PRISM software suite. Time-to-lysis was calculated as the time where the first derivative reaches 0 immediately following the initial observed population decline.

FIG. 22A depicts a comparison of the host range percent of phage cocktails with combinations of 3 to 6 different phages.

FIG. 22B depicts the host range percent of a the LBP-EC01 cocktail.

FIG. 23A depicts a representative example of a plaquing assay of LBP-EC01 phage cocktail against 352 clinical E. coli. Isolates. FIG. 23B depicts the summary of plaquing results across all strains tested.

### DETAILED DESCRIPTION

### Certain Terminology

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

Unless the context indicates otherwise, it is specifically intended that the various features of the disclosure described herein are able of being used in any combination. Moreover, the present disclosure also contemplates that in some embodiments, any feature or combination of features set forth herein are excluded or omitted. To illustrate, if the specification states that a composition comprises components A, B and C, it is specifically intended that any of A, B or C, or a combination thereof, are omitted and disclaimed singularly or in any combination.

One of skill in the art will understand the interchangeability of terms designating the various CRISPR-Cas systems and their components due to a lack of consistency in the literature and an ongoing effort in the art to unify such terminology.

As used in the description and the appended claims, the singular forms “a,” “an” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The term “about” as used herein when referring to a measurable value such as a dosage or time period and the like refers to variations of ±20%, ±10%, ±5%, ±1%, +0.5%, or even ±0.1% of the specified amount. As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise”, “comprises”, and “comprising”, “includes”, “including”, “have” and “having”, as used herein, specify the presence of the stated features, steps, operations, elements, and/or components, but do not preclude the presence or addition of one or more other features, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

The term “consists of” and “consisting of”, as used herein, excludes any features, steps, operations, elements, and/or components not otherwise directly stated. The use of “consisting of” limits only the features, steps, operations, elements, and/or components set forth in that clause and does exclude other features, steps, operations, elements, and/or components from the claim as a whole.

The terms “complementary” or “complementarity”, as used herein, refer to the natural binding of polynucleotides under permissive salt and temperature conditions by base-pairing. For example, the sequence “A-G-T” binds to the complementary sequence “T-C-A.” Complementarity between two single-stranded molecules is “partial,” in which only some of the nucleotides bind, or it is complete when total complementarity exists between the single stranded molecules. The degree of complementarity between nucleic acid strands has significant effects on the efficiency and strength of hybridization between nucleic acid strands.

“Complement” as used herein means 100% complementarity or identity with the comparator nucleotide sequence or it means less than 100% complementarity (e.g., about 80%, 81%, 82%, 83%, 84%, 85%, 86%, 87%, 88%, 89%, 90%, 91%, 92%, 93%, 94%, 95%, 96%, 97%, 98%, 99%, and the like, complementarity). Complement or complementable may also be used in terms of a “complement” to or “complementing” a mutation.

As used herein, the phrase “substantially identical,” or “substantial identity” in the context of two nucleic acid molecules, nucleotide sequences or protein sequences, refers to two or more sequences or subsequences that have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity refers to two or more sequences or subsequences that have at least about 70%, at least about 75%, at least about 80%, at least about 85%, at least about 90%, at least about 95, 96, 96, 97, 98, or 99% identity. For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences is to a full-length polynucleotide sequence or to a portion thereof, or to a longer polynucleotide sequence. In some instances, “Percent identity” is determined using BLASTX version 2.0 for translated nucleotide sequences and BLASTN version 2.0 for polynucleotide sequences.

As used herein, a “target nucleotide sequence” refers to the portion of a target gene (i.e., target region in the genome or the “protospacer sequence,” which is adjacent to a protospacer adjacent motif (PAM) sequence) that is fully complementary or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a CRISPR array.

As used herein, the term “protospacer adjacent motif” or “PAM” refers to a DNA sequence present on the target DNA molecule adjacent to the nucleotide sequence matching the spacer sequence. This motif is found in the target gene next to the region to which a spacer sequence binds as a result of being complementary to that region and identifies the point at which base pairing with the spacer nucleotide sequence begins. The exact PAM sequence that is required varies between each different CRISPR-Cas system. Non-limiting examples of PAMs include CCA, CCT, CCA, TTC, AAG, AGG, ATG, GAG, and/or CC. In some instances, in Type I systems, the PAM is located immediately 5′ to the sequence that matches the spacer, and thus is 3′ to the sequence that base pairs with the spacer nucleotide sequence, and is directly recognized by Cascade. In some instances, for B. halodurans Type I-C systems, the PAM is YYC, where Y can be either T or C. In some instances, for the P. aeruginosa Type I-C system, the PAM is TTC. Once a cognate protospacer and PAM are recognized, Cas3 is recruited, which then cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a—protospacer adjacent motif recognition domain at the C-terminus of Cas9). As used herein, the term “gene” refers to a nucleic acid molecule capable of being used to produce mRNA, tRNA, rRNA, miRNA, anti-microRNA, regulatory RNA, and the like. Genes may or may not be capable of being used to produce a functional protein or gene product. Genes include both coding and non-coding regions (e.g., introns, regulatory elements, promoters, enhancers, termination sequences and/or 5′ and 3′ untranslated regions). A gene is “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, the term “CRISPR phage”, “CRISPR enhanced phage”, and “crPhage” refers to a bacteriophage particle comprising bacteriophage DNA comprising at least one heterologous polynucleotide that encodes at least one component of a CRISPR-Cas system (e.g., CRISPR array, crRNA; e.g., P1 bacteriophage comprising an insertion of a targeting crRNA). In some embodiments, the polynucleotide encodes at least one transcriptional activator of a CRISPR-Cas system. In some embodiments, the polynucleotide encodes at least one component of an anti-CRISPR polypeptide of a CRISPR-Cas system.

By the terms “treat,” “treating,” or “treatment,” it is intended that the severity of the subject's condition is reduced or at least partially improved or modified and that some alleviation, mitigation or decrease in at least one clinical symptom is achieved, and/or there is a delay in the progression of the disease or condition, and/or delay of the onset of a disease or illness. With respect to an infection, a disease or a condition, the term refers to a decrease in the symptoms or other manifestations of the infection (including bacterial burden in the subject's tissues), disease or condition. In some embodiments, treatment provides a reduction in symptoms or other manifestations of the infection, disease or condition by at least about 5%, e.g., about 10%, 15%, 20%, 25%, 30%, 35%, 40%, 45%, 50% or more.

The terms “prevent,” “preventing,” and “prevention” (and grammatical variations thereof) refer to prevention and/or delay of the onset of an infection, disease, condition and/or a clinical symptom(s) in a subject and/or a reduction in the severity of the onset of the infection, disease, condition and/or clinical symptom(s) relative to what would occur in the absence of carrying out the methods disclosed herein prior to the onset of the disease, disorder and/or clinical symptom(s). Thus, in some embodiments, to prevent infection, food, surfaces, medical tools and devices are treated with compositions and by methods disclosed herein.

The terms with respect to an “infection”, “a disease”, or “a condition”, used herein, refer to any adverse, negative, or harmful physiological condition in a subject. In some embodiments, the source of an “infection”, “a disease”, or “a condition”, is the presence of a target bacterial population in and/or on a subject. In some embodiments, the bacterial population comprises one or more target bacterial species. In some embodiments, the one or more bacteria species in the bacterial population comprise one or more strains of one or more bacteria. In some embodiments, the target bacterial population causes an “infection”, “a disease”, or “a condition” that is acute or chronic. In some embodiments, the target bacterial population causes an “infection”, “a disease”, or “a condition” that is localized or systemic. In some embodiments, the target bacterial population causes an “infection”, “a disease”, or “a condition” that is idiopathic. In some embodiments, the target bacterial population causes an “infection”, “a disease”, or “a condition” that is acquired through means, including but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias.

The terms “individual”, or “subject” as used herein includes any animal that has or is susceptible to an infection, disease or condition involving bacteria. Thus, in some embodiments, subjects are mammals, avians, reptiles, amphibians, fish, crustaceans, or mollusks. Mammalian subjects include but are not limited to humans, non-human primates (e.g., gorilla, monkey, baboon, and chimpanzee, etc.), dogs, cats, goats, horses, pigs, cattle, sheep, and the like, and laboratory animals (e.g., rats, guinea pigs, mice, gerbils, hamsters, and the like). Avian subjects include but are not limited to chickens, ducks, turkeys, geese, quail, pheasants, and birds kept as pets (e.g., parakeets, parrots, macaws, cockatoos, canaries, and the like). Fish subjects include but are not limited to species used in aquaculture (e.g., tuna, salmon, tilapia, catfish, carp, trout, cod, bass, perch, snapper, and the like). Crustacean subjects include but are not limited to species used in aquaculture (e.g., shrimp, prawn, lobster, crayfish, crab and the like). Mollusk subjects include but are not limited to species used in aquaculture (e.g., abalone, mussel, oyster, clams, scallop and the like). In some embodiments, suitable subjects include both males and females and subjects of any age, including embryonic (e.g., in-utero or in-ovo), infant, juvenile, adolescent, adult and geriatric subjects. In some embodiments, a subject is a human.

As used here the term “isolated” in context of a nucleic acid sequence is a nucleic acid sequence that exists apart from its native environment.

As used herein, “expression cassette” means a recombinant nucleic acid molecule comprising a nucleotide sequence of interest (e.g., the recombinant nucleic acid molecules and CRISPR arrays disclosed herein), wherein the nucleotide sequence is operably associated with at least a control sequence (e.g., a promoter).

As used herein, “chimeric” refers to a nucleic acid molecule or a polypeptide in which at least two components are derived from different sources (e.g., different organisms, different coding regions).

As used herein, “selectable marker” means a nucleotide sequence that when expressed imparts a distinct phenotype to the host cell expressing the marker and thus allows such transformed cells to be distinguished from those that do not have the marker.

As used herein, “vector” refers to a composition for transferring, delivering or introducing a nucleic acid (or nucleic acids) into a cell.

As used herein, “pharmaceutically acceptable” means a material that is not biologically or otherwise undesirable, i.e., the material is administered to a subject without causing any undesirable biological effects such as toxicity.

As used herein the term “biofilm” means an accumulation of microorganisms embedded in a matrix of polysaccharide. Biofilms form on solid biological or non-biological surfaces and are medically important, accounting for over 80 percent of microbial infections in the body.

As used herein, the term “in vivo” is used to describe an event that takes place in a subject's body.

As used herein, the term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

### CRISPR/CAS Systems

CRISPR-Cas systems are naturally adaptive immune systems found in bacteria and archaea. The CRISPR system is a nuclease system involved in defense against invading phages and plasmids that provides a form of acquired immunity. There is a diversity of CRISPR-Cas systems based on the set of cas genes and their phylogenetic relationship. There are at least six different types (I through VI) where Type I represents over 50% of all identified systems in both bacteria and archaea. In some embodiments, a Type I, Type II, Type III, Type IV, Type V, or Type VI CRISPR-Cas system is used herein.

Type I systems are divided into seven subtypes including: Type I-A, Type I-B, Type I-C, Type I-D, Type I-E, Type I-F, and Type I-U. Type I CRISPR-Cas systems include a multi-subunit complex called Cascade (for complex associated with antiviral defense), Cas3 (a protein with nuclease, helicase, and exonuclease activity that is responsible for degradation of the target DNA), and CRISPR array encoding crRNA (stabilizes Cascade complex and directs Cascade and Cas3 to DNA target). Cascade forms a complex with the crRNA, and the protein-RNA pair recognizes its genomic target by complementary base pairing between the 5′ end of the crRNA sequence and a predefined protospacer. This complex is directed to homologous loci of pathogen DNA via regions encoded within the crRNA and protospacer-adjacent motifs (PAMs) within the pathogen genome. Base pairing occurs between the crRNA and the target DNA sequence leading to a conformational change. In the Type I-E system, the PAM is recognized by the CasA protein within Cascade, which then unwinds the flanking DNA to evaluate the extent of base pairing between the target and the spacer portion of the crRNA. Sufficient recognition leads Cascade to recruit and activate Cas3. Cas3 then nicks the non-target strand and begins degrading the strand in a 3′-to-5′ direction.

In the Type I-C system, the proteins Cas5, Cas8c, and Cas7 form the Cascade effector complex. Cas5 processes the pre-crRNA (which can take the form of a multi-spacer array, or a single spacer between two repeats) to produce individual crRNA(s) made up of a hairpin structure formed from the remaining repeat sequence and a linear spacer. The effector complex then binds to the processed crRNA and scans DNA to identify PAM sites. In the Type I-C system, the PAM is recognized by the Cas8c protein, which then acts to unwind the DNA duplex. If the sequence 3′ of the PAM matches the crRNA spacer that is bound to effector complex, a conformational change in the complex occurs and Cas3 is recruited to the site. Cas3 then nicks the non-target strand and begins degrading the DNA.

In some embodiments, the CRISPR-Cas system is endogenous to the target bacterium. In some embodiments, the CRISPR-Cas system is a Type I CRISPR-Cas system In some embodiments, the CRISPR-Cas system is a Type I-A CRISPR-Cas system. In some embodiments, the CRISPR-Cas system is a Type I-B CRISPR-Cas system. In some embodiments, the CRISPR-Cas system is a Type I-C CRISPR-Cas system. In some embodiments, the CRISPR-Cas system is a Type I-C CRISPR-Cas system derived from Pseudomonas aeruginosa. In some embodiments, the CRISPR-Cas system is a Type I-D CRISPR-Cas system. In some embodiments, the CRISPR-Cas system is a Type I-E CRISPR-Cas system. In some embodiments, the CRISPR-Cas system is a Type I-F CRISPR-Cas system. In some embodiments, the CRISPR-Cas system is a Type I-U CRISPR-Cas system. In some embodiments, the CRISPR-Cas system is a Type II CRISPR-Cas system. In some embodiments, the CRISPR-Cas system is a Type III CRISPR-Cas system. In some embodiments, the CRISPR-Cas system is a Type IV CRISPR-Cas system. In some embodiments, the CRISPR-Cas system is a Type V CRISPR-Cas system. In some embodiments, the CRISPR-Cas system is a Type VI CRISPR-Cas system.

In some embodiments, processing of a CRISPR-array disclosed herein includes, but is not limited to, the following processes: 1) transcription of the nucleic acid encoding a pre-crRNA, 2) recognition of the pre-crRNA by Cascade and/or specific members of Cascade, such as Cas6., and (3) processing of the pre-crRNA by Cascade or members of Cascade, such as Cas6, into mature crRNAs. In some embodiments, the mode of action for a Type I CRISPR system includes, but is not limited to, the following processes: 4) mature crRNA complexation with Cascade; 5) target recognition by the complexed mature crRNA/Cascade complex; and 6) nuclease activity at the target leading to DNA degradation.

CRISPR Phages

Disclosed herein, in certain embodiments, are bacteriophage compositions comprising CRISPR-Cas systems and methods of use thereof.

Bacteriophages or “phages” represent a group of bacterial viruses and are engineered or sourced from environmental sources. Individual bacteriophage host ranges are usually narrow, meaning, phages are highly specific to one strain or few strains of a bacterial species and this specificity makes them unique in their antibacterial action. Bacteriophages are bacterial viruses that rely on the host's cellular machinery to replicate. Bacteriophages are generally classified as virulent or temperate phages depending on their lifestyle. Virulent bacteriophages, also known as lytic bacteriophages, can only undergo lytic replication. Lytic bacteriophages infect a host cell, undergo numerous rounds of replication, and trigger cell lysis to release newly made bacteriophage particles. In some embodiments, the lytic bacteriophages disclosed herein retain their replicative ability. In some embodiments, the lytic bacteriophages disclosed herein retain their ability to trigger cell lysis. In some embodiments, the lytic bacteriophages disclosed herein retain both they replicative ability and the ability to trigger cell lysis. In some embodiments, the bacteriophages disclosed herein comprise a CRISPR array. In some embodiments, the CRISPR array does not affect the bacteriophages ability to replicate and/or trigger cell lysis. Temperate or lysogenic bacteriophages can undergo lysogeny in which the phage stops replicating and stably resides within the host cell, either integrating into the bacterial genome or being maintained as an extrachromosomal plasmid. Temperate phages can also undergo lytic replication similar to their lytic bacteriophage counterparts. Whether a temperate phage replicates lytically or undergoes lysogeny upon infection depends on a variety of factors including growth conditions and the physiological state of the cell. A bacterial cell that has a lysogenic phage integrated into its genome is referred to as a lysogenic bacterium or lysogen. Exposure to adverse conditions may trigger reactivation of the lysogenic phage, termination of the lysogenic state and resumption of lytic replication by the phage. This process is called induction. Adverse conditions which favor the termination of the lysogenic state include desiccation, exposure to UV or ionizing radiation, and exposure to mutagenic chemicals. This leads to the expression of the phage genes, reversal of the integration process, and lytic multiplication. In some embodiments, the temperate bacteriophages disclosed herein are rendered lytic. The term “lysogeny gene” refers to any gene whose gene product promotes lysogeny of a temperate phage. Lysogeny genes can directly promote, as in the case of integrase proteins that facilitate integration of the bacteriophage into the host genome. Lysogeny genes can also indirectly promote lysogeny as in the case of CI transcriptional regulators which prevent transcription of genes required for lytic replication and thus favor maintenance of lysogeny.

Bacteriophages package and deliver synthetic DNA using three general approaches. Under the first approach, the synthetic DNA is recombined into the bacteriophage genome in a targeted manner, which usually involves a selectable marker. Under the second approach, restriction sites within the phage are used to introduce synthetic DNA in-vitro. Under the third approach, a plasmid generally encoding the phage packaging sites and lytic origin of replication is packaged as part of the assembly of the bacteriophage particle. The resulting plasmids have been coined “phagemids.”

Phages are limited to a given bacterial strain for evolutionary reasons. In some cases, injecting their genetic material into an incompatible strain is counterproductive. Phages have therefore evolved to specifically infect a limited cross-section of bacterial strains. However, some phages have been discovered that inject their genetic material into a wide range of bacteria. The classic example is the P1 phage, which has been shown to inject DNA in a range of gram-negative bacteria.

Disclosed herein, in some embodiments, are bacteriophages comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic. In some embodiments, the bacteriophage is a temperate bacteriophage. In some embodiments, the bacteriophage is rendered lytic by removal, replacement, or inactivation of a lysogenic gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the bacteriophage is rendered lytic by the removal of a regulatory element of a lysogeny gene. In some embodiments, the bacteriophage is rendered lytic by the removal of a promoter of a lysogeny gene. In some embodiments, the bacteriophage is rendered lytic by the removal of a functional element of a lysogeny gene. In some embodiments, the lysogenic gene is an activator gene. In some embodiments, the lysogenic gene is cII gene. In some embodiments, the lysogenic gene is lexA gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, the bacteriophage is rendered lytic via a second CRISPR array comprising a second spacer directed to a lysogenic gene. In some embodiments, the bacteriophage is rendered lytic by the insertion of one or more lytic genes. In some embodiments, the bacteriophage is rendered lytic by the insertion of one or more genes that contribute to the induction of a lytic cycle. In some embodiments, the bacteriophage is rendered lytic by altering the expression of one or more genes that contribute to the induction of a lytic cycle. In some embodiments, the bacteriophage phenotypically changes from a lysogenic bacteriophage to a lytic bacteriophage. In some embodiments, the phenotypic change is via a self-targeting CRISPR-Cas system to render a bacteriophage lytic since it is incapable of lysogeny. In some embodiments, the self-targeting CRISPR-Cas comprises a self-targeting crRNA from the prophage genome and kills lysogens. In some embodiments, the bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI)). In some embodiments, the bacteriophage that is rendered lytic is prevented from reverting to lysogenic state. In some embodiments, the bacteriophage that is rendered lytic is prevented from reverting back to lysogenic state by way of introducing an additional CRISPR array. In some embodiments, the bacteriophage does not confer any new properties onto the target bacterium beyond cellular death cause by lytic activity of the bacteriophage and/or the activity of the CRISPR array. Further disclosed herein, in some embodiments, are temperate bacteriophages comprising a first nucleic acid sequence encoding a first spacer sequence or a crRNA transcribed therefrom, wherein the first spacer sequence is complementary to a target nucleotide sequence from a target gene in a target bacterium, provided the bacteriophage is rendered lytic. In some embodiments, the bacteriophage infects multiple bacterial strains. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the target bacterium. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. In some embodiments, the target nucleotide sequence is in a non-essential gene. In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a target bacterium. In some embodiments, the spacer sequence is complementary to a target nucleotide sequence of a sequence present in the target bacterium. In some embodiments, the spacer sequence is complementary to a target nucleotide sequence that comprises all or a part of a promoter sequence of the essential gene. In some embodiments, the first nucleic acid sequence comprises a first CRISPR array comprising at least one repeat sequence. In some embodiments, the at least one repeat sequence is operably linked to the first spacer sequence at either its 5′ end or its 3′ end.

In some embodiments, the bacteriophage is T4 E. coli bacteriophage. In some embodiments, the bacteriophage is T7 E. coli bacteriophage. In some embodiments, the bacteriophage is T7M E. coli bacteriophage.

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle. In some embodiments, the non-essential gene to be removed and/or replaced from the bacteriophage is the hoc gene from a T4 E. coli bacteriophage. In some embodiments, the non-essential gene to be removed and/or replaced includes gp0.7, gp4.3, gp4.5, gp4.7, or any combination thereof from a T7 E. coli bacteriophage. In some embodiments, the non-essential gene to be removed and/or replaced is gp0.6, gp0.65, gp0.7, gp4.3, gp4.5, or any combination thereof from a T7M E. coli bacteriophage.

In some embodiments, a plurality of bacteriophages are used together. In some embodiments, the plurality of bacteriophages used together targets the same or different bacteria within a sample or subject. In some embodiments, the bacteriophages used together comprises T4 phage, T7 phage, T7M phage, or any combination of bacteriophages described herein.

In some embodiments, bacteriophages of interest are obtained from environmental sources. or commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

In some embodiments, the nucleic acid is inserted into the bacteriophage genome. In some embodiments, the nucleic acid comprises a crArray, a Cas system, or a combination thereof. In some embodiments, the nucleic acid is inserted into the bacteriophage genome at a transcription terminator site at the end of an operon of interest. In some embodiments, the nucleic acid is inserted into the bacteriophage genome as a replacement for one or more removed non-essential genes. In some embodiments, the nucleic acid is inserted into the bacteriophage genome as a replacement for one or more removed lysogenic genes. In some embodiments, the replacement of non-essential and/or lysogenic genes with the nucleic acid enhances the lytic activity of the bacteriophage. In some embodiments, the replacement of non-essential and/or lysogenic genes with the nucleic acid renders a lysogenic bacteriophage lytic.

In some embodiments, the nucleic acid is introduced into the bacteriophage genome at a first location while one or more non-essential and/or lysogenic genes are separately removed and/or inactivated from the bacteriophage genome at a separate location. In some embodiments, the removal of one or more non-essential and/or lysogenic genes renders a lysogenic bacteriophage into a lytic bacteriophage. Similarly, in some embodiments, one or more lytic genes are introduced into the bacteriophage so as to render a non-lytic, lysogenic bacteriophage into a lytic bacteriophage.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

Disclosed herein, in certain embodiments, are nucleic acid sequences comprising (a) a first CRISPR array designed to be operable with a first Type I CRISPR-Cas system, and (b) a second CRISPR array designed to be operable with a second Type I CRISPR-Cas system, wherein the first Type I CRISPR-Cas system and the second Type I CRISPR-Cas system are different Type I CRISPR-Cas systems.

Also disclosed herein, are bacteriophages comprising a nucleic acid sequence comprising (a) a first CRISPR array designed to be operable with a first Type I CRISPR-Cas system, or (b) a leuO coding sequence. In some embodiments, the bacteriophages comprise a nucleic acid sequence comprising (a) a first CRISPR array designed to be operable with a first Type I CRISPR-Cas system, and (b) a leuO coding sequence. In some embodiments, the nucleic acid sequence further comprises (c) a second CRISPR array designed to be operable with a second Type I CRISPR-Cas system.

### CRISPR Array

In some embodiments, the first CRISPR array (crArray) and the second CRISPR array are on the same nucleic acid sequence. In some embodiments, the first CRISPR array and/or the second CRISPR array encodes a processed, mature crRNA. In some embodiments, the mature crRNA is introduced into a phage or a target bacterium. In some embodiments, an endogenous or exogenous Cas6 processes the first CRISPR array and/or the second CRISPR array into mature crRNA. In some embodiments, an exogenous Cas6 is introduced into the phage. In some embodiments, the phage comprises an exogenous Cas6. In some embodiments, an exogenous Cas6 is introduced into a target bacterium.

In some embodiments, the first CRISPR array comprises a spacer sequence. In some embodiments, the second CRISPR array comprises a spacer sequence. In some embodiments, the first CRISPR array further comprises at least one repeat sequence. In some embodiments, the second CRISPR array further comprises at least one repeat sequence. In some embodiments, the at least one repeat sequence is operably linked to the spacer sequence at either its 5′ end or its 3′ end. In some embodiments, first CRISPR array and/or the second CRISPR array is of any length and comprises any number of spacer nucleotide sequences alternating with repeat nucleotide sequences necessary to achieve the desired level of killing of a target bacterium by targeting one or more essential genes. In some embodiments, the first CRISPR array and/or the second CRISPR array comprises, consists essentially of, or consists of 1 to about 100 spacer nucleotide sequences, each linked on its 5′ end and its 3′ end to a repeat nucleotide sequence. In some embodiments, first CRISPR array and/or the second CRISPR array comprises, consists essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

### Spacer Sequence

In some embodiments, the spacer sequence is complementary to a target nucleotide sequence in a target bacterium. In some embodiments, the target nucleotide sequence is a coding region. In some embodiments, the coding region is an essential gene. In some embodiments, the coding region is a nonessential gene. In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a target bacterium. In some embodiments, the spacer sequence is complementary to a target nucleotide sequence of a sequence present in the target bacterium. In some embodiments, the spacer sequence is complementary to a target nucleotide sequence that comprises all or a part of a promoter sequence of the essential gene. In some embodiments, the spacer sequence comprises one, two, three, four, or five mismatches as compared to the target nucleotide sequence. In some embodiments, the mismatches are contiguous. In some embodiments, the mismatches are noncontiguous. In some embodiments, the spacer sequence has 70% complementarity to a target nucleotide sequence. In some embodiments, the spacer sequence has 80% complementarity to a target nucleotide sequence. In some embodiments, the spacer sequence is 85%, 90%, 95%, 96%, 97%, 98%, 99% or 100% complementarity to a target nucleotide sequence. In some embodiments, the spacer sequence has 100% complementarity to the target nucleotide sequence. In some embodiments, the spacer sequence has complete complementarity or substantial complementarity over a region of a target nucleotide sequence that are at least about 8 nucleotides to about 150 nucleotides in length. In some embodiments, a spacer sequence has complete complementarity or substantial complementarity over a region of a target nucleotide sequence that is at least about 20 nucleotides to about 100 nucleotides in length. In some embodiments, the 5 ‘ region of the spacer sequence is 100% complementary to a target nucleotide sequence while the 3’ region of the spacer is substantially complementary to the target nucleotide sequence and therefore the overall complementarity of the spacer sequence to the target nucleotide sequence is less than 100%. For example, in some embodiments, the first 7, 8, 9, 10, 11, 12, 13, 14, 15, 16 nucleotides in the 3′ region of a 20 nucleotide spacer sequence (seed region) is 100% complementary to the target nucleotide sequence, while the remaining nucleotides in the 5′ region of the spacer sequence are substantially complementary (e.g., at least about 70% complementary) to the target nucleotide sequence. In some embodiments, the first 7 to 12 nucleotides of the 3′ end of the spacer sequence is 100% complementary to the target nucleotide sequence, while the remaining nucleotides in the 5′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleotide sequence. In some embodiments, the first 7 to 10 nucleotides in the 3′ end of the spacer sequence is 75%-99% complementary to the target nucleotide sequence, while the remaining nucleotides in the 5′ region of the spacer sequence are at least about 50% to about 99% complementary to the target nucleotide sequence. In some embodiments, the first 7 to 10 nucleotides in the 3′ end of the spacer sequence is 100% complementary to the target nucleotide sequence, while the remaining nucleotides in the 5′ region of the spacer sequence are substantially complementary (e.g., at least about 70% complementary) to the target nucleotide sequence. In some embodiments, the first 10 nucleotides (within the seed region) of the spacer sequence is 100% complementary to the target nucleotide sequence, while the remaining nucleotides in the 5′ region of the spacer sequence are substantially complementary (e.g., at least about 70% complementary) to the target nucleotide sequence. In some embodiment, the 5′ region of a spacer sequence (e.g., the first 8 nucleotides at the 5′ end, the first 10 nucleotides at the 5′ end, the first 15 nucleotides at the 5′ end, the first 20 nucleotides at the 5′ end) have about 75% complementarity or more (75% to about 100% complementarity) to the target nucleotide sequence, while the remainder of the spacer sequence have about 50% or more complementarity to the target nucleotide sequence. In some embodiments, the first 8 nucleotides at the 5′ end of the spacer sequence have 100% complementarity to the target nucleotide sequence or have one or two mutations and therefore is about 88% complementary or about 75% complementary to the target nucleotide sequence, respectively, while the remainder of the spacer nucleotide sequence is at least about 50% or more complementary to the target nucleotide sequence.

In some embodiments, the spacer sequence is about 15 nucleotides to about 150 nucleotides in length. In some embodiments, the spacer nucleotide sequence is about 15 nucleotides to about 100 nucleotides in length (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides or more). In some embodiments, the spacer nucleotide sequence is a length of about 8 to about 150 nucleotides, about 8 to about 100 nucleotides, about 8 to about 50 nucleotides, about 8 to about 40 nucleotides, about 8 to about 30 nucleotides, about 8 to about 25 nucleotides, about 8 to about 20 nucleotides, about 10 to about 150 nucleotides, about 10 to about 100 nucleotides, about 10 to about 80 nucleotides, about 10 to about 50 nucleotides, about 10 to about 40, about 10 to about 30, about 10 to about 25, about 10 to about 20, about 15 to about 150, about 15 to about 100, about 15 to about 50, about 15 to about 40, about 15 to about 30, about 20 to about 150 nucleotides, about 20 to about 100 nucleotides, about 20 to about 80 nucleotides, about 20 to about 50 nucleotides, about 20 to about 40, about 20 to about 30, about 20 to about 25, at least about 8, at least about 10, at least about 15, at least about 20, at least about 25, at least about 30, at least about 32, at least about 35, at least about 40, at least about 44, at least about 50, at least about 60, at least about 70, at least about 80, at least about 90, at least about 100, at least about 110, at least about 120, at least about 130, at least about 140, at least about 150 nucleotides in length, or more, and any value or range therein. In some embodiments, the P. aeruginosa Type I-C Cas system has a spacer length of about 30 to 39 nucleotides, about 31 to about 38 nucleotides, about 32 to about 37 nucleotides, about 33 to about 36 nucleotides, about 34 to about 35 nucleotides, or about 35 nucleotides In some embodiments, the P. aeruginosa Type I-C Cas system has a spacer length of about 34 nucleotides. In some embodiments, the P. aeruginosa Type I-C Cas system has a spacer length of at least about 10, at least about 15, at least about 20, at least about 21, at least about 22, at least about 23, at least about 24, at least about 25, at least about 26, at least about 27, at least about 29, at least about 29, at least about 30, at least about 31, at least about 32, at least about 33, at least about 34, at least about, at least about 35, at least about 36, at least about 37, at least about 38, at least about 39, at least about 20, at least about 41, at least about 42, at least about 43, at least about 44, at least about 45, or more than about 45 nucleotides.

In some embodiments, the identity of two or more spacer sequences of the first CRISPR and/or the second CRISPR array is the same. In some embodiments, the identity of two or more spacer sequences of the first CRISPR and/or the second CRISPR array is different. In some embodiments, the identity of two or more spacer sequences of the first CRISPR and/or the second CRISPR array is different but are complementary to one or more target nucleotide sequences. In some embodiments, the identity of two or more spacer nucleotide sequences of the first CRISPR and/or the second CRISPR array is different and are complementary to one or more target nucleotide sequences that are overlapping sequences. In some embodiments, the identity of two or more spacer nucleotide sequences of the first CRISPR and/or the second CRISPR array is different and are complementary to one or more target nucleotide sequences that are not overlapping sequences. In some embodiments, the target nucleotide sequence is about 10 to about 40 consecutive nucleotides in length located immediately adjacent to a PAM sequence (PAM sequence located immediately 3′ of the target region) in the genome of the organism. In some embodiments, a target nucleotide sequence is located adjacent to or flanked by a PAM (protospacer adjacent motif).

The PAM sequence is found in the target gene next to the region to which a spacer sequence binds as a result of being complementary to that region and identifies the point at which base pairing with the spacer nucleotide sequence begins. The exact PAM sequence that is required varies between each different CRISPR-Cas system and is identified through established bioinformatics and experimental procedures. Non-limiting examples of PAMs include CCA, CCT, CCG, TTC, AAG, AGG, ATG, GAG, and/or CC. For Type I systems, the PAM is located immediately 5′ to the sequence that matches the spacer, and thus is 3′ to the sequence that base pairs with the spacer nucleotide sequence, and is directly recognized by Cascade. Once a protospacer is recognized, Cascade generally recruits the endonuclease Cas3, which cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a—protospacer adjacent motif recognition domain at the C-terminus of Cas9)

In some embodiments, the target nucleotide sequence in the bacterium to be killed is any essential target nucleotide sequence of interest. In some embodiments, the target nucleotide sequence is a non-essential sequence. In some embodiments, a target nucleotide sequence comprises, consists essentially of or consist of all or a part of a nucleotide sequence encoding a promoter, or a complement thereof, of the essential gene. In some embodiments, the spacer nucleotide sequence is complementary to a promoter, or a part thereof, of the essential gene. In some embodiments, the target nucleotide sequence comprises all or a part of a nucleotide sequence located on a coding or a non-coding strand of the essential gene. In some embodiments, the target nucleotide sequence comprises all or a part of a nucleotide sequence located on a coding of a transcribed region of the essential gene.

In some embodiments, the essential gene is any gene of an organism that is critical for its survival. However, being essential is highly dependent on the circumstances in which an organism lives. For instance, a gene required to digest starch is only essential if starch is the only source of energy. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the target bacterium. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. In some embodiments, a non-essential gene is any gene of an organism that is not critical for survival. However, being non-essential is highly dependent on the circumstances in which an organism lives.

In some embodiments, non-limiting examples of the target nucleotide sequence of interest includes a target nucleotide sequence encoding a transcriptional regulator, a translational regulator, a polymerase gene, a metabolic enzyme, a transporter, an RNase, a protease, a DNA replication enzyme, a DNA modifying or degrading enzyme, a regulatory RNA, a transfer RNA, or a ribosomal RNA. In some embodiments, the target nucleotide sequence is from a gene involved in cell-division, cell structure, metabolism, motility, pathogenicity, virulence, or antibiotic resistance. In some embodiments, the target nucleotide sequence is from a hypothetical gene whose function is not yet characterized. Thus, for example, these genes are any genes from any bacterium.

The appropriate spacer sequences for a full-construct phage may be identified by locating a search set of representative genomes, searching the genomes with relevant parameters, and determining the quality of a spacer for use in a CRISPR engineered phage.

First, a suitable search set of representative genomes is located and acquired for the organism/species/target of interest. The set of representative genomes may be found in a variety of databases, including without limitations the NCBI GenBank or the PATRIC database. NCBI GenBank is one of the largest databases available and contains a mixture of reference and submitted genomes for nearly every organism sequenced to date. Specifically, for pathogenic prokaryotes, the PATRIC (Pathosystems Resource Integration Center) database provides an additional comprehensive resource of genomes and provides a focus on clinically relevant strains and genomes relevant to a drug product. Both of the above databases allow for bulk downloading of genomes via FTP (File Transfer Protocol) servers, enabling rapid and programmatic dataset acquisition

Next, the genomes are searched with relevant parameters to locate suitable spacer sequences. Genomes may be read from start to end, in both the forward and reverse complement orientations, to locate contiguous stretches of DNA that contain a PAM (Protospacer Adjacent Motif) site. The spacer sequence will be the N-length DNA sequence 3′ or 5′ adjacent to the PAM site (depending on the CRISPR system type), where N is specific to the Cas system of interest and is generally known ahead of time. Characterizing the PAM sequence and spacer sequences may be performed during the discovery and initial research of a Cas system. Every observed PAM-adjacent spacer may be saved to a file and/or database for downstream use. The exact PAM sequence that is required varies between each different CRISPR-Cas system and is identified through established bioinformatics and experimental procedures.

Next, the quality of a spacer for use in a CRISPR engineered phage is determined. Each observed spacer may be evaluated to determine how many of the evaluated genomes they are present in. The observed spacers may be evaluated to see how many times they may occur in each given genome. Spacers that occur in more than one location per genome may be advantageous because the Cas system may not be able to recognize the target site if a mutation occurs, and each additional “backup” site increases the likelihood that a suitable, non-mutated target location will be present. The observed spacers may be evaluated to determine whether they occur in functionally annotated regions of the genome. If such information is available, the functional annotations may be further evaluated to determine whether those regions of the genome are “essential” for the survival and function of the organism. By focusing on spacers that occur in all, or nearly all, evaluated genomes of interest (>=99%), the spacer selection may be broadly applicable to many targeted genomes. Provided a large selection pool of conserved spacers exists, preference may be given to spacers that occur in regions of the genome that have known function, with higher preference given if those genomic regions are “essential” for survival and occur more than 1 time per genome.

### Repeat Nucleotide Sequences

In some embodiments, a repeat nucleotide sequence of the first CRISPR and/or the second CRISPR array comprises a nucleotide sequence of any known repeat nucleotide sequence of a Type I CRISPR-Cas system. In some embodiments, a repeat nucleotide sequence is of a synthetic sequence comprising the secondary structure of a native repeat from a Type I CRISPR-Cas system (e.g., an internal hairpin). In some embodiments, the repeat nucleotide sequences are distinct from one another based on the known repeat nucleotide sequences of a CRISPR-Cas system. In some embodiments, the repeat nucleotide sequences are each composed of distinct secondary structures of a native repeat from a CRISPR-Cas system (e.g., an internal hairpin). In some embodiments, the repeat nucleotide sequences are a combination of distinct repeat nucleotide sequences operable with a CRISPR-Cas system.

In some embodiments, the spacer nucleotide sequence is linked at its 5′ end to a first repeat sequence and linked at its 3′ end to a second repeat sequence to form a repeat-spacer-repeat sequence. In some embodiments, the spacer sequence is linked at its 5′ end to the 3′ end of a first repeat sequence and is linked at its 3′ end to the 5′ of a second repeat sequence where the spacer sequence and the second repeat sequence are repeated to form a repeat-(spacer-repeat)n sequence such that n is any integer from 1 to 100. In some embodiments, a repeat-(spacer-repeat)n sequence comprises, consists essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

In some embodiments, the repeat sequence is identical to or substantially identical to a repeat sequence from a wild-type CRISPR loci. In some embodiments, the repeat sequence is a repeat sequence found in Table 3. In some embodiments, the repeat sequence is a sequence described herein. In some embodiments, the repeat sequence comprises a portion of a wild type repeat sequence (e.g., 1, 2, 3, 4, 5, 6, 7, 8, 9, 10, 11, 12, 13, 14, 15 or more contiguous nucleotides of a wild type repeat sequence). In some embodiments, the repeat sequence comprises, consists essentially of, or consists of at least one nucleotide (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, or more nucleotides, or any range therein). In some embodiments, the repeat sequence comprises, consists essentially of, or consists of no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 nucleotides. In some embodiments, the repeat sequence comprises about 20 to 40, 21 to 40, 22 to 40 23 to 40, 24 to 40, 25 to 40, 26 to 40, 27 to 40, 28 to 40, 29 to 40, 30 to 30, 31 to 40, 32 to 40, 33 to 40, 34 to 40, 35 to 40, 36 to 40, 37 to 40, 38 to 40, 39 to 40, 20 to 39, 20 to 38, 20 to 37, 20 to 36, 20 to 35, 20 to 34, 20 to 33, 20 to 32, 20 to 31, 20 to 30, 20 to 29, 20 to 28, 20 to 26, 20 to 25, 20 to 24, 20 to 23, 20 to 22, or 20 to 21 nucleotides. In some embodiments, the repeat sequence comprises about 20 to 35, 21 to 35, 22 to 35 23 to 35, 24 to 35, 25 to 35, 26 to 35, 27 to 35, 28 to 35, 29 to 35, 30 to 30, 31 to 35, 32 to 35, 33 to 35, 34 to 35, 25 to 40, 25 to 39, 25 to 38, 25 to 37, 25 to 36, 25 to 35, 25 to 34, 25 to 33, 25 to 32, 25 to 31, 25 to 30, 25 to 29, 25 to 28, 25 to 26 nucleotides. In some embodiments, the system is a P. aeruginosa Type I-C Cas system. In some embodiments, the P. aeruginosa Type I-C Cas system has a repeat length of about 25 to 38 nucleotides.

### Transcriptional Activators

In some embodiments, the nucleic acid sequence further comprises a transcriptional activator. In some embodiments, the transcriptional activator encoded regulates the expression of genes of interest within the target bacterium. In some embodiments, the transcriptional activator activates the expression of genes of interest within the target bacterium whether exogenous or endogenous. In some embodiments, the transcriptional activator activates the expression genes of interest within the target bacterium by disrupting the activity of one or more inhibitory elements within the target bacterium. In some embodiments, the inhibitory element comprises a transcriptional repressor. In some embodiments, the inhibitory element comprises a global transcriptional repressor. In some embodiments the inhibitory element is a histone-like nucleoid-structuring (H-NS) protein or homologue or functional fragment thereof. In some embodiments, the inhibitory element is a leucine responsive regulatory protein (LRP). In some embodiments, the inhibitory element is a CodY protein.

In some bacteria, the CRISPR-Cas system is poorly expressed and considered silent under most environmental conditions, for example laboratory conditions. In these bacteria, the regulation of the CRISPR-Cas system is the result of the activity of transcriptional regulators, for example histone-like nucleoid-structuring (H-NS) protein which is widely involved in transcriptional regulation of the host genome. H-NS exerts control over host transcriptional regulation by multimerization along AT-rich sites resulting in DNA bending. In some bacteria, such as E. coli, the regulation of the Type I CRISPR-Cas3 operon is regulated by H-NS.

Similarly, in some bacteria, the repression of the CRISPR-Cas system is controlled by an inhibitory element, for example the leucine responsive regulatory protein (LRP). LRP has been implicated in binding to upstream and downstream regions of the transcriptional start sites. Notably, the activity of LRP in regulating expression of the CRISPR-Cas system varies from bacteria to bacteria. Unlike, H-NS which has broad inter-species repression activity, LRP has been shown to differentially regulate the expression of the host CRISPR-Cas system. As such, in some instances, LRP reflects a host-specific means of regulating CRISPR-Cas system expression in different bacteria.

In some instances, the repression of CRISPR-Cas system is also controlled by inhibitory element CodY. CodY is a GTP-sensing transcriptional repressor that acts through DNA binding. The intracellular concentration of GTP acts as an indicator for the environmental nutritional status. Under normal culture conditions, GTP is abundant and binds with CodY to repress transcriptional activity. However, as GTP concentrations decreases, CodY becomes less active in binding DNA, thereby allowing transcription of the formerly repressed genes to occur. As such, CodY acts as a stringent global transcriptional repressor.

In some embodiments, the transcriptional activator is a LeuO polypeptide, any homolog or functional fragment thereof, a leuO coding sequence, or an agent that upregulates LeuO. In some embodiments, the transcriptional activator comprises any ortholog or functional equivalent of LeuO. In some bacteria, LeuO acts in opposition to H-NS by acting as a global transcriptional regulator that responds to environmental nutritional status of a bacterium. Under normal conditions, LeuO is poorly expressed. However, under amino acid starvation and/or reaching of the stationary phase in the bacterial life cycle, LeuO is upregulated. Increased expression of LeuO leads to it antagonizing H-NS at overlapping promoter regions to effect gene expression. Overexpression of LeuO upregulates the expression of the CRISPR-Cas system. In E. coli and S. typhimurium, LeuO drives increased expression of the casABCDE operon which has predicted LeuO and H-NS binding sequences upstream of CasA.

In some embodiments, the expression of LeuO leads to disruption of an inhibitory element. In some embodiments, the disruption of an inhibitory element due to expression of LeuO removes the transcriptional repression of a CRISPR-Cas system. In some embodiments, the expression of LeuO removes transcriptional repression of a CRISPR-Cas system due to activity of H-NS. In some embodiments, the disruption of an inhibitory element due to the expression of LeuO causes an increase in the expression of a CRISPR-Cas system. In some embodiments, the increase in the expression of a CRISPR-Cas system due to the disruption of an inhibitory element caused by the expression of LeuO causes an increase in the CRISPR-Cas processing of a nucleic acid sequence comprising a CRISPR array. In some embodiments, the increase in the expression of a CRISPR-Cas system due to the disruption of an inhibitory element by the expression of LeuO causes an increase in the CRISPR-Cas processing of a nucleic acid sequence comprising a CRISPR array so as to increase the level of lethality of the CRISPR array against a bacterium. In some embodiments, transcriptional activator causes increase activity of a bacteriophage and/or the CRISPR-Cas system.

### Regulatory Elements

In some embodiments, the nucleic acid sequences are operatively associated with a variety of promoters, terminators and other regulatory elements for expression in various organisms or cells. In some embodiments, the nucleic acid sequence further comprises a leader sequence. In some embodiments, the nucleic acid sequence further comprises a promoter sequence. In some embodiments, at least one promoter and/or terminator is operably linked to the CRISPR array. Any promoter useful with this disclosure is used and includes, for example, promoters functional with the organism of interest as well as constitutive, inducible, developmental regulated, tissue-specific/preferred-promoters, and the like, as disclosed herein. A regulatory element as used herein is endogenous or heterologous. In some embodiments, an endogenous regulatory element derived from the subject organism is inserted into a genetic context in which it does not naturally occur (e.g. a different position in the genome than as found in nature), thereby producing a recombinant or non-native nucleic acid.

In some embodiments, expression of the nucleic acid sequence is constitutive, inducible, temporally regulated, developmentally regulated, or chemically regulated. In some embodiments, the expression of the nucleic acid sequence is made constitutive, inducible, temporally regulated, developmentally regulated, or chemically regulated by operatively linking the nucleic acid sequence to a promoter functional in an organism of interest. In some embodiments, repression is made reversible by operatively linking the nucleic acid sequence to an inducible promoter that is functional in an organism of interest. The choice of promoter disclosed herein varies depending on the quantitative, temporal and spatial requirements for expression, and also depending on the host cell to be transformed.

Exemplary promoters for use with the methods, bacteriophages and compositions disclosed herein include promoters that are functional in bacteria. For example, L-arabinose inducible (araBAD, P_{BAD}) promoter, any lac promoter, L-rhamnose inducible (rhaPBAD) promoter, T7 RNA polymerase promoter, trc promoter, tac promoter, lambda phage promoter (p_{L}p_{L}-9G-50), anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, a-amylase (Pamy), Ptms, P43 (comprised of two overlapping RNA polymerase a factor recognition sites, σA, σB), Ptms, P43, rplK-rplA, ferredoxin promoter, and/or xylose promoter. In some embodiments, the promoter is a BBa_J23102 promoter. In some embodiments, the promoter works in a broad range of bacteria, such as BBa_J23104, BBa_J23109. In some embodiments the promoter is derived from the target bacterium, such as endogenous CRISPR promoter, endogenous Cas operon promoter, p16, plpp, or ptat. In some embodiments, the promoter is a phage promoter, such as the promoter for gp105 or gp245.

In some embodiments, inducible promoters are used. In some embodiments, chemical-regulated promoters are used to modulate the expression of a gene in an organism through the application of an exogenous chemical regulator. The use of chemically regulated promoters enables RNAs and/or the polypeptides encoded by the nucleic acid sequence to be synthesized only when, for example, an organism is treated with the inducing chemicals. In some embodiments where a chemical-inducible promoter is used, the application of a chemical induces gene expression. In some embodiments wherein a chemical-repressible promoter is used, the application of the chemical represses gene expression. In some embodiments, the promoter is a light-inducible promoter, where application of specific wavelengths of light induces gene expression. In some embodiments, a promoter is a light-repressible promoter, where application of specific wavelengths of light represses gene expression.

### Nucleic Acid Sequence

In some embodiments, the nucleic acid sequence is at least 70%, at least 75%, at least 80%, at least 85%, at least 90%, at least 95%, at least 96%, at least 97%, at least 98%, at least 99%, or 100% identical to a nucleic acid sequence set forth as SEQ ID NO: 1. In some embodiments, the nucleotide acid sequence is isolated. In some instances, an isolated nucleic acid sequence exists in a purified form that is at least partially separated from at least some of the other components of the naturally occurring organism or virus, for example, the cell or viral structural components or other polypeptides or nucleic acids commonly found associated with the polynucleotide. In some embodiments, the isolated nucleic acid sequence is at least about 1%, 5%, 10%, 20%, 30%, 40%, 50%, 60%, 70%, 80%, 90%, 95%, or more pure.

Nucleic acid sequence
[SEQ ID NO: 1]
GATAATTAGTGCTGCGGGTAGGTAAGGATAAAAAAG
GGTGGCAGCAGGAGATTGAGATGGTTTTGCTTTAT
TAACAACGGGCTAAACGTGTAGTATTTGAGCTAGC
GAATTCGAGCTCGGTACCACCTCGAGTTCCCCGCG
CCAGCGGGGATAAACCGCTGAAGTAGAAAAACGTG
TTACAGCATCAGTCGAGTTCCCCGCGCCAGCGGGG
ATAAACCGGGAGAGAGTGAGCGATCCTCCGTTAAC
ATAGTTCACTGCCGTACAGGCAGCTTAGAAAGTAT
TATTCGACGGCGGTGGGATTGCTTCACGTTCACTG
CCGTACAGGCAGCTTAGAAAATCAAAATTGCTGTC
TGCCAGGTGATCGCTTTGACAGCTAGCTCAGTCCT
AGGTACTGTGCTAGCATAAAGGAGGTAAATAATGC
CAGAGGTACAAACAGATCATCCAGAGACAGCGGAG
TTAAGCAAACCACAGCTACGCATGGTCGATCTCAA
CTTATTAACCGTTTTCGATGCCGTGATGCAGGAGC
AAAACATTACTCGTGCCGCTCATGTTCTGGGAATG
TCGCAACCTGCGGTCAGTAACGCTGTTGCACGCCT
GAAGGTGATGTTTAATGACGAGCTTTTTGTTCGTT
ATGGCCGTGGTATTCAACCGACTGCTCGCGCATTT
CAACTTTTTGGTTCAGTTCGTCAGGCATTGCAACT
AGTACAAAATGAATTGCCTGGTTCAGGTTTTGAAC
CCGCGAGCAGTGAACGTGTATTTCATCTTTGTGTT
TGCAGCCCGTTAGACAGCATTCTGACCTCGCAGAT
TTATAATCACATTGAGCAGATTGCGCCAAATATAC
ATGTTATGTTCAAGTCGTCATTAAATCAGAACACT
GAACATCAGCTGCGTTATCAGGAAACGGAGTTTGT
GATTAGTTATGAGGACTTCCATCGTCCTGAATTTA
CCAGCGTACCATTATTTAAAGATGAAATGGTGCTG
GTAGCCAGCAAAAATCATCCAACAATTAAGGGCCC
GTTACTGAAACATGATGTTTATAACGAACAACATG
CGGCGGTTTCGCTCGATCGTTTCGCGTCATTTAGT
CAACCTTGGTATGACACGGTAGATAAGCAAGCCAG
TATCGCGTATCAGGGCATGGCAATGATGAGCGTAC
TTAGCGTGGTGTCGCAAACGCATTTGGTCGCTATT
GCGCCGCGTTGGCTGGCTGAAGAGTTCGCTGAATC
CTTAGAATTACAGGTATTACCGCTGCCGTTAAAAC
AAAACAGCAGAACCTGTTATCTCTCCTGGCATGAA
GCTGCCGGGCGCGATAAAGGCCATCAGTGGATGGA
AGAGCAATTAGTCTCAATTTGCAAACGCTAA

### Expression Cassette

In some embodiments, the nucleic acid sequence is an expression cassette or in an expression cassette. In some embodiments, the expression cassettes are designed to express the nucleic acid sequence disclosed herein.

In some embodiments, an expression cassette comprising a nucleic acid sequence of interest is chimeric, meaning that at least one of its components is heterologous with respect to at least one of its other components. In some embodiments, an expression cassette is naturally occurring but has been obtained in a recombinant form useful for heterologous expression.

In some embodiments, an expression cassette includes a transcriptional and/or translational termination region (i.e. termination region) that is functional in the selected host cell. In some embodiments, termination regions are responsible for the termination of transcription beyond the heterologous nucleic acid sequence of interest and for correct mRNA polyadenylation. In some embodiments, the termination region is native to the transcriptional initiation region, is native to the operably linked nucleic acid sequence of interest, is native to the host cell, or is derived from another source (i.e., foreign or heterologous to the promoter, to the nucleic acid sequence of interest, to the host, or any combination thereof). In some embodiments, terminators are operably linked to the nucleic acid sequence disclosed herein.

In some embodiments, an expression cassette includes a nucleotide sequence for a selectable marker. In some embodiments, the nucleotide sequence encodes either a selectable or a screenable marker, depending on whether the marker confers a trait that is selected for by chemical means, such as by using a selective agent (e.g. an antibiotic), or on whether the marker is simply a trait that one identifies through observation or testing, such as by screening (e.g., fluorescence).

### Vectors

In addition to expression cassettes, the nucleic acid sequences disclosed herein (e.g. nucleic acid sequence comprising a CRISPR array) are used in connection with vectors. A vector comprises a nucleic acid molecule comprising the nucleotide sequence(s) to be transferred, delivered or introduced. Non-limiting examples of general classes of vectors include, but are not limited to, a viral vector, a plasmid vector, a phage vector, a phagemid vector, a cosmid vector, a fosmid vector, a bacteriophage, an artificial chromosome, or an agrobacterium binary vector in double or single stranded linear or circular form which may or may not be self-transmissible or mobilizable. A vector transforms prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms. In some embodiments, a shuttle vector replicates in actinomycetes and bacteria and/or eukaryotes. In some embodiments, the nucleic acid in the vector are under the control of, and operably linked to, an appropriate promoter or other regulatory elements for transcription in a host cell. In some embodiments, the vector is a bi-functional expression vector which functions in multiple hosts.

### Codon Optimization

In some embodiments, the nucleic acid sequence is codon optimized for expression in any species of interest. Codon optimization involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species-specific codon usage table with the codons present in the native polynucleotide sequences. Codon optimization of a nucleotide sequence results in a nucleotide sequence having less than 100% identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function as that encoded by the original nucleotide sequence. In some embodiments, the nucleic acid sequences of this disclosure are codon optimized for expression in the organism/species of interest.

### Transformation

In some embodiments, the nucleic acid sequence, and/or expression cassettes disclosed herein are expressed transiently and/or stably incorporated into the genome of a host organism. In some embodiments, a the nucleic acid sequence and/or expression cassettes disclosed herein is introduced into a cell by any method known to those of skill in the art. Exemplary methods of transformation include transformation via electroporation of competent cells, passive uptake by competent cells, chemical transformation of competent cells, as well as any other electrical, chemical, physical (mechanical) and/or biological mechanism that results in the introduction of nucleic acid into a cell, including any combination thereof. In some embodiments, transformation of a cell comprises nuclear transformation. In some embodiments, transformation of a cell comprises plasmid transformation and conjugation.

In some embodiments, when more than one nucleic acid sequence is introduced, the nucleotide sequences are assembled as part of a single nucleic acid construct, or as separate nucleic acid constructs, and are located on the same or different nucleic acid constructs. In some embodiments, nucleotide sequences are introduced into the cell of interest in a single transformation event, or in separate transformation events.

### Type I CRISPR-Cas System

In some embodiments, the first Type I CRISPR-Cas system is a Type I-A system, Type I-B system, Type I-C system, Type I-D system, Type I-E system, or Type I-F system. In some embodiments, the first Type I CRISPR-Cas system is a Type I-A system. In some embodiments, the first Type I CRISPR-Cas system is a Type I-B system. In some embodiments, the first Type I CRISPR-Cas system is a Type I-C system. In some embodiments, the first Type I CRISPR-Cas system is a Type I-D system. In some embodiments, the first Type I CRISPR-Cas system is a Type I-E system. In some embodiments, the first Type I CRISPR-Cas system is a Type I-F system. In some embodiments, the second Type I CRISPR-Cas system is a Type I-A system, Type I-B system, Type I-C system, Type I-D system, Type I-E system, or Type I-F system. In some embodiments, the second Type I CRISPR-Cas system is a Type I-A system. In some embodiments, the second Type I CRISPR-Cas system is a Type I-B system. In some embodiments, the second Type I CRISPR-Cas system is a Type I-C system. In some embodiments, the second Type I CRISPR-Cas system is a Type I-D system. In some embodiments, the second Type I CRISPR-Cas system is a Type I-E system. In some embodiments, the second Type I CRISPR-Cas system is a Type I-F system. In some embodiments, the first Type I CRISPR-Cas system is a Type I-E system and the second Type I CRISPR-Cas system is a Type I-F system. In some embodiments, the first Type I CRISPR-Cas system is a Type I-F system and the second Type I CRISPR-Cas system is a Type I-E system. In some embodiments, the first Type I CRISPR-Cas system is endogenous to the target bacterium. In some embodiments, the second Type I CRISPR-Cas system is endogenous to the target bacterium. In some embodiments, the first Type I CRISPR-Cas system and the second Type I CRISPR-Cas system is endogenous to the target bacterium. In some embodiments, the endogenous Type I CRISPR-Cas system comprises Cascade polypeptides. Type I Cascade polypeptides process CRISPR arrays to produce a processed RNA that is then used to bind the complex to a target sequence that is complementary to the spacer in the processed RNA. In some embodiments, the Type I Cascade complex is a Type I-A Cascade polypeptides, a Type I-B Cascade polypeptides, a Type I-C Cascade polypeptides, a Type I-D Cascade polypeptides, a Type I-E Cascade polypeptides, a Type I-F Cascade polypeptides, or a Type I-U Cascade polypeptides.

In some embodiments, the Type I Cascade complex comprises: (a) a nucleotide sequence encoding a Cas7 (Csa2) polypeptide, a nucleotide sequence encoding a Cas8a1 (Csx13) polypeptide or a Cas8a2 (Csx9) polypeptide, a nucleotide sequence encoding a Cas5 polypeptide, a nucleotide sequence encoding a Csa5 polypeptide, a nucleotide sequence encoding a Cas6a polypeptide, a nucleotide sequence encoding a Cas3′ polypeptide, and a nucleotide sequence encoding a Cas3″ polypeptide having no nuclease activity (Type I-A); (b) a nucleotide sequence encoding a Cas6b polypeptide, a nucleotide sequence encoding a Cas8b (Csh1) polypeptide, a nucleotide sequence encoding a Cas7 (Csh2) polypeptide, and a nucleotide sequence encoding a Cas5 polypeptide (Type I-B); (c) a nucleotide sequence encoding a Cas5d polypeptide, a nucleotide sequence encoding a Cas8c (Csd1) polypeptide, and a nucleotide sequence encoding a Cas7 (Csd2) polypeptide (Type I-C); (d) a nucleotide sequence encoding a Cas1 Od (Csc3) polypeptide, a nucleotide sequence encoding a Csc2 polypeptide, a nucleotide sequence encoding a Csc1 polypeptide, and a nucleotide sequence encoding a Cas6d polypeptide (Type I-D); (e) a nucleotide sequence encoding a Cse1 (CasA) polypeptide, a nucleotide sequence encoding a Cse2 (CasB) polypeptide, a nucleotide sequence encoding a Cas7 (CasC) polypeptide, a nucleotide sequence encoding a Cas5 (CasD) polypeptide, and a nucleotide sequence encoding a Cas6e (CasE) polypeptide (Type I-E); and/or (f) a nucleotide sequence encoding a Cys1 polypeptide, a nucleotide sequence encoding a Cys2 polypeptide, a nucleotide sequence encoding a Cas7 (Cys3) polypeptide, and a nucleotide sequence encoding a Cas6f polypeptide (Type I-F).

### Target Bacterium

In some embodiments, the target bacterium comprises one or more species of the target bacterium. In some embodiments, the target bacterium comprises one or more strains of the target bacterium. In some embodiments, the target bacterium is E. coli. In some embodiments, the E. coli is a multidrug-resistant (MDR) strain. In some embodiments, the E. coli is an extended spectrum beta-lactamase (ESBL) strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the E. coli strain comprises Ec 527, also referred to interchangeably as NC101 and Ec LFP527.

In some embodiments, the target bacterium causes an infection or disease. In some embodiments, the infection or disease is acute or chronic. In some embodiments, the infection or disease is localized or systemic. In some embodiments, infection or disease is idiopathic. In some embodiments, the infection or disease is acquired through means including, but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias. In some embodiments, the target bacterium causes urinary tract infection. In some embodiments, the E. coli causes and/or exacerbates urinary tract infection. In some embodiments, the target bacterium causes and/or exacerbates an inflammatory disease. In some embodiments, the target bacterium causes and/or exacerbates inflammatory bowel disease (IBD). In some embodiments, the E. coli causes and/or exacerbates inflammatory bowel disease (IBD). In some embodiments, the E. coli causes and/or exacerbates Crohn's disease. In some embodiments, the E. coli causes and/or exacerbates Ulcerative colitis.

### Bacteriophage

In some embodiments, the bacteriophage is an obligate lytic bacteriophage. In some embodiments, the bacteriophage is a temperate bacteriophage with a lysogeny gene removed, replaced, or inactivated, thereby rendering the bacteriophage lytic. In some embodiments, the bacteriophages include, but are not limited to, T4, T7, T7M, M13, p0046-9, p0033s-6, p0071-16, p0033L-10, p00ex-2, p0031-8, or p004k-5. In some embodiments, the phage is crT7M. In some embodiments, the phage is crT4. In some embodiments, the phage is crT7. In some embodiments, the phage is crM13. In some embodiments, the phage is p0046-9. In some embodiments, the phage is p0033s-6. In some embodiments, the phage is p0071-16. In some embodiments, the phage is p0033L-10. In some embodiments, the phage is p00ex-2. In some embodiments, the phage is p0031-8. In some embodiments, the phage is p004k-5. In some embodiments, the bacteriophage is T4 E. coli bacteriophage. In some embodiments, the bacteriophage is T7 E. coli bacteriophage. In some embodiments, the bacteriophage is T7M E. coli bacteriophage. In some embodiments, the bacteriophage is M13 E. coli bacteriophage. In some embodiments, the bacteriophage is p0046-9 E. coli bacteriophage. In some embodiments, the bacteriophage is p0033s-6 E. coli bacteriophage. In some embodiments, the bacteriophage is p0071-16 E. coli bacteriophage. In some embodiments, the bacteriophage is p0033L-10 E. coli bacteriophage. In some embodiments, the bacteriophage is p00ex-2 E. coli bacteriophage. In some embodiments, the bacteriophage is p0031-8 E. coli bacteriophage. In some embodiments, the bacteriophage is p004k-5 E. coli bacteriophage. In some embodiments, the bacteriophage comprises a phage listed in Table 1A.

TABLE 1A
Bacteriophage by accession number

| | ATCC |
|---|---|
| | Accession |
| Phage name | number |
| p0031-8 | PTA-126315 |
| p0033L-10 | PTA-126316 |
| p0033s-6 | PTA-126317 |
| p0045-9 | PTA-126318 |
| p004k-5 | PTA-126319 |
| p0071-16 | PTA-126320 |
| p0078-4 | PTA-126321 |
| p00dd-1 | PTA-126322 |
| p00E8-3 | PTA-126323 |
| p00Ex-2 | PTA-126324 |
| p00Jc-2 | PTA-126325 |

In some embodiments, bacteriophages of interest are obtained from environmental sources or from commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

### Insertion Sites

In some embodiments, the insertion of the nucleic acid sequence into a bacteriophage does not disrupt the lytic activity of the bacteriophage. In some embodiments, the insertion of the nucleic acid sequence into a bacteriophage preserves the lytic activity of the bacteriophage. In some embodiments, the nucleic acid sequence is inserted into the bacteriophage genome. In some embodiments, the nucleic acid sequence is inserted into the bacteriophage genome at a transcription terminator site at the end of an operon of interest. In some embodiments, the nucleic acid sequence is inserted into the bacteriophage genome as a replacement for one or more removed non-essential genes. In some embodiments, the nucleic acid sequence is inserted into the bacteriophage genome as a replacement for one or more removed lysogenic genes. In some embodiments, the replacement of non-essential and/or lysogenic genes with the nucleic acid sequence does not affect the lytic activity of the bacteriophage. In some embodiments, the replacement of non-essential and/or lysogenic genes with the nucleic acid sequence preserves the lytic activity of the bacteriophage. In some embodiments, the replacement of non-essential and/or lysogenic genes with the nucleic acid sequence enhances the lytic activity of the bacteriophage. In some embodiments, the replacement of non-essential and/or lysogenic genes with the nucleic acid sequence renders a lysogenic bacteriophage lytic.

In some embodiments, the nucleic acid sequence is introduced into the bacteriophage genome at a first location while one or more non-essential and/or lysogenic genes are separately removed and/or inactivated from the bacteriophage genome at a separate location. In some embodiments, the nucleic acid sequence is introduced into the bacteriophage at a first location while one or more non-essential and/or lysogenic genes are separately removed and/or inactivated from the bacteriophage genome at multiple separate locations. In some embodiments, the removal and/or inactivation of one or more non-essential and/or lysogenic genes does not affect the lytic activity of the bacteriophage. In some embodiments, the removal and/or inactivation of one or more non-essential and/or lysogenic genes preserves the lytic activity of the bacteriophage. In some embodiments, the removal of one or more non-essential and/or lysogenic genes renders a lysogenic bacteriophage into a lytic bacteriophage.

In some embodiments, the bacteriophage is a temperate bacteriophage which has been rendered lytic by any of the aforementioned means. In some embodiments, a temperate bacteriophage is rendered lytic by the removal, replacement, or inactivation of one or more lysogenic genes. In some embodiments, the lytic activity of the bacteriophage is due to the removal, replacement, or inactivation of at least one lysogeny gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the lysogenic gene is an activator gene. In some embodiments, the lysogenic gene is cII gene. In some embodiments, the lysogenic gene is lexA gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, a temperate bacteriophage is rendered lytic by the insertion of one or more lytic genes. In some embodiments, a temperate bacteriophage is rendered lytic by the insertion of one or more genes that contribute to the induction of a lytic cycle. In some embodiments, a temperate bacteriophage is rendered lytic by altering the expression of one or more genes that contribute to the induction of a lytic cycle. In some embodiments, a temperate bacteriophage phenotypically changes from a lysogenic bacteriophage to a lytic bacteriophage. In some embodiments, a temperate bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI). In some embodiments, a temperate bacteriophage that is rendered lytic is prevented from reverting to lysogenic state. In some embodiments, a temperate bacteriophage that is rendered lytic is prevented from reverting back to lysogenic state by way of the self-targeting activity of the first introduced CRISPR array. In some embodiments, a temperate bacteriophage that is rendered lytic is prevented from reverting back to lysogenic state by way of introducing an additional CRISPR array. In some embodiments, the bacteriophage does not confer any new properties onto the target bacterium beyond cellular death cause by lytic activity of the bacteriophage and/or the activity of the first or second CRISPR array.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

### Non-Essential Gene

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle. In some embodiments, the non-essential gene to be removed and/or replaced from the bacteriophage is the hoc gene from a T4 E. coli bacteriophage. In some embodiments, the non-essential gene to be removed and/or replaced include gp0.7, gp4.3, gp4.5, gp4.7, or any combination thereof from a T7 E. coli bacteriophage. In some embodiments, the non-essential gene to be removed and/or replaced is gp0.6, gp0.65, gp0.7, gp4.3, gp4.5, or any combination thereof from a T7M E. coli bacteriophage.

In some embodiments, the bacteriophage is at least 70%, at least 75%, at least 80%, at least 85%, at least 90%, at least 95%, at least 96%, at least 97%, at least 98%, at least 99%, or 100% identical to a T7M bacteriophage comprising a CRISPR array. In some embodiments, the bacteriophage is at least 70%, at least 75%, at least 80%, at least 85%, at least 90%, at least 95%, at least 96%, at least 97%, at least 98%, at least 99%, or 100% identical to a T7M bacteriophage comprising SEQ ID NO: 1. In some embodiments, the bacteriophage is at least 70%, at least 75%, at least 80%, at least 85%, at least 90%, at least 95%, at least 96%, at least 97%, at least 98%, at least 99%, or 100% identical to a T4 bacteriophage comprising a CRISPR array. In some embodiments, the bacteriophage is at least 70%, at least 75%, at least 80%, at least 85%, at least 90%, at least 95%, at least 96%, at least 97%, at least 98%, at least 99%, or 100% identical to a T4 bacteriophage comprising SEQ ID NO: 1 In some embodiments, the bacteriophage is at least 70%, at least 75%, at least 80%, at least 85%, at least 90%, at least 95%, at least 96%, at least 97%, at least 98%, at least 99%, or 100% identical to a T7 bacteriophage comprising a CRISPR array. In some embodiments, the bacteriophage is at least 70%, at least 75%, at least 80%, at least 85%, at least 90%, at least 95%, at least 96%, at least 97%, at least 98%, at least 99%, or 100% identical to a T7 bacteriophage comprising SEQ ID NO:1. In some embodiments, the bacteriophage is at least 70%, at least 75%, at least 80%, at least 85%, at least 90%, at least 95%, at least 96%, at least 97%, at least 98%, at least 99%, or 100% identical to a M13 bacteriophage comprising a CRISPR array. In some embodiments, the bacteriophage is at least 70%, at least 75%, at least 80%, at least 85%, at least 90%, at least 95%, at least 96%, at least 97%, at least 98%, at least 99%, or 100% identical to a M13 bacteriophage comprising SEQ ID NO:1. In some embodiments, the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126317. In some embodiments, the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126320. In some embodiments, the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126316. In some embodiments, the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126324. In some embodiments, the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126315. In some embodiments, the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319. In some embodiments, the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126321. In some embodiments, the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126322. In some embodiments, the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126323. In some embodiments, the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126325.

### Phage Cocktails

Also disclosed herein, in certain embodiments, are compositions comprising a plurality of bacteriophages disclosed herein. In some embodiments, the plurality of bacteriophages used together targets the same or different target bacterium within a sample or subject. In some embodiments, the bacteriophages used together comprises any combination of bacteriophages disclosed herein.

In some embodiments, the composition comprises at least two, at least three, at least four, at least five, at least six, at least seven, at least eight, at least nine, at least ten, or at least 11 bacteriophages selected from a list comprising: (i) the bacteriophage at least 70%, at least 75%, at least 80%, at least 85%, at least 90%, at least 95%, at least 96%, at least 97%, at least 98%, at least 99%, or 100% identical to a T7M bacteriophage comprising a CRISPR array, (ii) the bacteriophage at least 70%, at least 75%, at least 80%, at least 85%, at least 90%, at least 95%, at least 96%, at least 97%, at least 98%, at least 99%, or 100% identical to a T4 bacteriophage comprising a CRISPR array, (iii) the bacteriophage at least 70%, at least 75%, at least 80%, at least 85%, at least 90%, at least 95%, at least 96%, at least 97%, at least 98%, at least 99%, or 100% identical to a T7 bacteriophage comprising a CRISPR array, (iv) the bacteriophage at least 70%, at least 75%, at least 80%, at least 85%, at least 90%, at least 95%, at least 96%, at least 97%, at least 98%, at least 99%, or 100% identical to a M13 bacteriophage comprising a CRISPR array, (v) the bacteriophage at least 70%, at least 75%, at least 80%, at least 85%, at least 90%, at least 95%, at least 96%, at least 97%, at least 98%, at least 99%, or 100% identical to a bacteriophage, (vi) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit Patent Deposit number PTA-126317, (vii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit Patent Deposit number PTA-126320, (viii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit Patent Deposit number PTA-126316, (ix) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit Patent Deposit number PTA-126324, (x) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit Patent Deposit number PTA-126315, (xi) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319, (xii) the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126321, (xiii) the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126322, (xiv) the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126323, and (xv) the bacteriophage is at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126325.

In some embodiments, the composition comprises (a) the bacteriophage at least 70%, at least 75%, at least 80%, at least 85%, at least 90%, at least 95%, at least 96%, at least 97%, at least 98%, at least 99%, or 100% identical to a T7M bacteriophage comprising a CRISPR array, and (b) at least one more bacteriophage selected from the list comprising: (i) the bacteriophage at least 70%, at least 75%, at least 80%, at least 85%, at least 90%, at least 95%, at least 96%, at least 97%, at least 98%, at least 99%, or 100% identical to a T4 bacteriophage comprising a CRISPR array, (ii) the bacteriophage at least 70%, at least 75%, at least 80%, at least 85%, at least 90%, at least 95%, at least 96%, at least 97%, at least 98%, at least 99%, or 100% identical a T7 bacteriophage comprising a CRISPR array, (iii) the bacteriophage at least 70%, at least 75%, at least 80%, at least 85%, at least 90%, at least 95%, at least 96%, at least 97%, at least 98%, at least 99%, or 100% identical to a M13 bacteriophage comprising a CRISPR array, (iv) the bacteriophage at least 70%, at least 75%, at least 80%, at least 85%, at least 90%, at least 95%, at least 96%, at least 97%, at least 98%, at least 99%, or 100% identical to a bacteriophage disclosed herein, (v) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit Patent Deposit number PTA-126317, (vi) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126320, (vii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126316, (viii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126324, (ix) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126315, and (x) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319.

In some embodiments, the composition comprises (a) the bacteriophage at least 70%, at least 75%, at least 80%, at least 85%, at least 90%, at least 95%, at least 96%, at least 97%, at least 98%, at least 99%, or 100% identical a T4 bacteriophage comprising a CRISPR array, and (b) at least one more bacteriophage selected from the list comprising: (i) the bacteriophage at least 70%, at least 75%, at least 80%, at least 85%, at least 90%, at least 95%, at least 96%, at least 97%, at least 98%, at least 99%, or 100% identical to a T7M bacteriophage comprising a CRISPR array, (ii) the bacteriophage at least 70%, at least 75%, at least 80%, at least 85%, at least 90%, at least 95%, at least 96%, at least 97%, at least 98%, at least 99%, or 100% identical to a T7 bacteriophage comprising a CRISPR array, (iii) the bacteriophage at least 70%, at least 75%, at least 80%, at least 85%, at least 90%, at least 95%, at least 96%, at least 97%, at least 98%, at least 99%, or 100% identical to a M13 bacteriophage comprising a CRISPR array, (iv) the bacteriophage at least 70%, at least 75%, at least 80%, at least 85%, at least 90%, at least 95%, at least 96%, at least 97%, at least 98%, at least 99%, or 100% identical to a disclosed herein, (v) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126317, (vi) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126320, (vii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126316, (viii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126324, (ix) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126315, and (x) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319.

In some embodiments, the composition comprises (a) the bacteriophage at least 70%, at least 75%, at least 80%, at least 85%, at least 90%, at least 95%, at least 96%, at least 97%, at least 98%, at least 99%, or 100% identical to a T7 bacteriophage comprising a CRISPR array, and (b) at least one more bacteriophage selected from the list comprising: (i) the bacteriophage at least 70%, at least 75%, at least 80%, at least 85%, at least 90%, at least 95%, at least 96%, at least 97%, at least 98%, at least 99%, or 100% identical to a T7M bacteriophage comprising a CRISPR array, (ii) the bacteriophage at least 70%, at least 75%, at least 80%, at least 85%, at least 90%, at least 95%, at least 96%, at least 97%, at least 98%, at least 99%, or 100% identical to a T4 bacteriophage comprising a CRISPR array, (iii) the bacteriophage at least 70%, at least 75%, at least 80%, at least 85%, at least 90%, at least 95%, at least 96%, at least 97%, at least 98%, at least 99%, or 100% identical to a M13 bacteriophage comprising a CRISPR array, (iv) the bacteriophage at least 70%, at least 75%, at least 80%, at least 85%, at least 90%, at least 95%, at least 96%, at least 97%, at least 98%, at least 99%, or 100% identical to a bacteriophage disclosed herein, (v) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126317, (vi) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126320, (vii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126316, (viii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126324, (ix) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126315, and (x) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319.

In some embodiments, the composition comprises (a) the bacteriophage at least 70%, at least 75%, at least 80%, at least 85%, at least 90%, at least 95%, at least 96%, at least 97%, at least 98%, at least 99%, or 100% identical a M13 bacteriophage comprising a CRISPR array, and (b) at least one more bacteriophage selected from the list comprising: (i) the bacteriophage at least 70%, at least 75%, at least 80%, at least 85%, at least 90%, at least 95%, at least 96%, at least 97%, at least 98%, at least 99%, or 100% identical to a T7M bacteriophage comprising a CRISPR array, (ii) the bacteriophage at least 70%, at least 75%, at least 80%, at least 85%, at least 90%, at least 95%, at least 96%, at least 97%, at least 98%, at least 99%, or 100% identical to a T4 bacteriophage comprising a CRISPR array, (iii) the bacteriophage at least 70%, at least 75%, at least 80%, at least 85%, at least 90%, at least 95%, at least 96%, at least 97%, at least 98%, at least 99%, or 100% identical to a T7 bacteriophage comprising a CRISPR array, (iv) the bacteriophage at least 70%, at least 75%, at least 80%, at least 85%, at least 90%, at least 95%, at least 96%, at least 97%, at least 98%, at least 99%, or 100% identical to a disclosed herein, (v) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126317, (vi) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126320, (vii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126316, (viii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126324, (ix) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126315, and (x) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319.

In some embodiments, the composition comprises (a) the bacteriophage at least 70%, at least 75%, at least 80%, at least 85%, at least 90%, at least 95%, at least 96%, at least 97%, at least 98%, at least 99%, or 100% identical to a bacteriophage disclosed herein, and (b) at least one more bacteriophage selected from the list comprising: (i) the bacteriophage at least 70%, at least 75%, at least 80%, at least 85%, at least 90%, at least 95%, at least 96%, at least 97%, at least 98%, at least 99%, or 100% identical to a T7M bacteriophage comprising a CRISPR array, (ii) the bacteriophage at least 70%, at least 75%, at least 80%, at least 85%, at least 90%, at least 95%, at least 96%, at least 97%, at least 98%, at least 99%, or 100% identical to a T4 bacteriophage comprising a CRISPR array, (iii) the bacteriophage at least 70%, at least 75%, at least 80%, at least 85%, at least 90%, at least 95%, at least 96%, at least 97%, at least 98%, at least 99%, or 100% identical to a T7 bacteriophage comprising a CRISPR array, (iv) the bacteriophage at least 70%, at least 75%, at least 80%, at least 85%, at least 90%, at least 95%, at least 96%, at least 97%, at least 98%, at least 99%, or 100% identical a M13 bacteriophage comprising a CRISPR array, (v) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126317, (vi) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126320, (vii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126316, (viii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126324, (ix) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126315, and (x) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319.

In some embodiments, the composition comprises (a) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126317, and (b) at least one more bacteriophage selected from the list comprising: (i) the bacteriophage at least 70%, at least 75%, at least 80%, at least 85%, at least 90%, at least 95%, at least 96%, at least 97%, at least 98%, at least 99%, or 100% identical to a T7 bacteriophage comprising a CRISPR array, (ii) the bacteriophage at least 70%, at least 75%, at least 80%, at least 85%, at least 90%, at least 95%, at least 96%, at least 97%, at least 98%, at least 99%, or 100% identical to a T4 bacteriophage comprising a CRISPR array, (iii) the bacteriophage at least 70%, at least 75%, at least 80%, at least 85%, at least 90%, at least 95%, at least 96%, at least 97%, at least 98%, at least 99%, or 100% identical to a T7 bacteriophage comprising a CRISPR array, (iv) the bacteriophage at least 70%, at least 75%, at least 80%, at least 85%, at least 90%, at least 95%, at least 96%, at least 97%, at least 98%, at least 99%, or 100% identical to a M13 bacteriophage comprising a CRISPR array, (v) the bacteriophage at least 70%, at least 75%, at least 80%, at least 85%, at least 90%, at least 95%, at least 96%, at least 97%, at least 98%, at least 99%, or 100% identical to a bacteriophage disclosed herein, (vi) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126320, (vii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126316, (viii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126324, (ix) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126315, and (x) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319.

In some embodiments, the composition comprises (a) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126320, and (b) at least one more bacteriophage selected from the list comprising: (i) the bacteriophage at least 70%, at least 75%, at least 80%, at least 85%, at least 90%, at least 95%, at least 96%, at least 97%, at least 98%, at least 99%, or 100% identical to a T7M bacteriophage comprising a CRISPR array, (ii) the bacteriophage at least 70%, at least 75%, at least 80%, at least 85%, at least 90%, at least 95%, at least 96%, at least 97%, at least 98%, at least 99%, or 100% identical to a T4 bacteriophage comprising a CRISPR array, (iii) the bacteriophage at least 70%, at least 75%, at least 80%, at least 85%, at least 90%, at least 95%, at least 96%, at least 97%, at least 98%, at least 99%, or 100% identical to a T7 bacteriophage comprising a CRISPR array, (iv) the bacteriophage at least 70%, at least 75%, at least 80%, at least 85%, at least 90%, at least 95%, at least 96%, at least 97%, at least 98%, at least 99%, or 100% identical to a M13 bacteriophage comprising a CRISPR array, (v) the bacteriophage at least 70%, at least 75%, at least 80%, at least 85%, at least 90%, at least 95%, at least 96%, at least 97%, at least 98%, at least 99%, or 100% identical to a bacteriophage disclosed herein, (vi) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126317, (vii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126316, (viii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126324, (ix) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126315, and (x) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319.

In some embodiments, the composition comprises (a) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126316, and (b) at least one more bacteriophage selected from the list comprising: (i) the bacteriophage at least 70%, at least 75%, at least 80%, at least 85%, at least 90%, at least 95%, at least 96%, at least 97%, at least 98%, at least 99%, or 100% identical to a T7M bacteriophage comprising a CRISPR array, (ii) the bacteriophage at least 70%, at least 75%, at least 80%, at least 85%, at least 90%, at least 95%, at least 96%, at least 97%, at least 98%, at least 99%, or 100% identical to a T4 bacteriophage comprising a CRISPR array, (iii) the bacteriophage at least 70%, at least 75%, at least 80%, at least 85%, at least 90%, at least 95%, at least 96%, at least 97%, at least 98%, at least 99%, or 100% identical to a T7 bacteriophage comprising a CRISPR array, (iv) the bacteriophage at least 70%, at least 75%, at least 80%, at least 85%, at least 90%, at least 95%, at least 96%, at least 97%, at least 98%, at least 99%, or 100% identical to a M13 bacteriophage comprising a CRISPR array, (v) the bacteriophage at least 70%, at least 75%, at least 80%, at least 85%, at least 90%, at least 95%, at least 96%, at least 97%, at least 98%, at least 99%, or 100% identical to a bacteriophage disclosed herein, (vi) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126317, (vii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126320, (viii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126324, (ix) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126315, and (x) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319.

In some embodiments, the composition comprises (a) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126324, and (b) at least one more bacteriophage selected from the list comprising: (i) the bacteriophage at least 70%, at least 75%, at least 80%, at least 85%, at least 90%, at least 95%, at least 96%, at least 97%, at least 98%, at least 99%, or 100% identical to a T7M bacteriophage comprising a CRISPR array, (ii) the bacteriophage at least 70%, at least 75%, at least 80%, at least 85%, at least 90%, at least 95%, at least 96%, at least 97%, at least 98%, at least 99%, or 100% identical to a T4 bacteriophage comprising a CRISPR array, (iii) the bacteriophage at least 70%, at least 75%, at least 80%, at least 85%, at least 90%, at least 95%, at least 96%, at least 97%, at least 98%, at least 99%, or 100% identical to a T7 bacteriophage comprising a CRISPR array, (iv) the bacteriophage at least 70%, at least 75%, at least 80%, at least 85%, at least 90%, at least 95%, at least 96%, at least 97%, at least 98%, at least 99%, or 100% identical to a M13 bacteriophage comprising a CRISPR array, (v) the bacteriophage at least 70%, at least 75%, at least 80%, at least 85%, at least 90%, at least 95%, at least 96%, at least 97%, at least 98%, at least 99%, or 100% identical to a bacteriophage disclosed herein, (vi) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126317, (vii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126320, (viii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126316, (ix) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126315, and (x) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319.

In some embodiments, the composition comprises (a) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126315, and (b) at least one more bacteriophage selected from the list comprising: (i) the bacteriophage at least 70%, at least 75%, at least 80%, at least 85%, at least 90%, at least 95%, at least 96%, at least 97%, at least 98%, at least 99%, or 100% identical to a T7M bacteriophage comprising a CRISPR array, (ii) the bacteriophage at least 70%, at least 75%, at least 80%, at least 85%, at least 90%, at least 95%, at least 96%, at least 97%, at least 98%, at least 99%, or 100% identical to a T4 bacteriophage comprising a CRISPR array, (iii) the bacteriophage at least 70%, at least 75%, at least 80%, at least 85%, at least 90%, at least 95%, at least 96%, at least 97%, at least 98%, at least 99%, or 100% identical to a T7 bacteriophage comprising a CRISPR array, (iv) the bacteriophage at least 70%, at least 75%, at least 80%, at least 85%, at least 90%, at least 95%, at least 96%, at least 97%, at least 98%, at least 99%, or 100% identical to a M13 bacteriophage comprising a CRISPR array, (v) the bacteriophage at least 70%, at least 75%, at least 80%, at least 85%, at least 90%, at least 95%, at least 96%, at least 97%, at least 98%, at least 99%, or 100% identical to a bacteriophage disclosed herein, (vi) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126317, (vii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126320, (viii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126316, (ix) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126324, and (x) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319.

In some embodiments, the composition comprises (a) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319, and (b) at least one more bacteriophage selected from the list comprising: (i) the bacteriophage at least 70%, at least 75%, at least 80%, at least 85%, at least 90%, at least 95%, at least 96%, at least 97%, at least 98%, at least 99%, or 100% identical to a T7M bacteriophage comprising a CRISPR array, (ii) the bacteriophage at least 70%, at least 75%, at least 80%, at least 85%, at least 90%, at least 95%, at least 96%, at least 97%, at least 98%, at least 99%, or 100% identical to a T4 bacteriophage comprising a CRISPR array, (iii) the bacteriophage at least 70%, at least 75%, at least 80%, at least 85%, at least 90%, at least 95%, at least 96%, at least 97%, at least 98%, at least 99%, or 100% identical to a T7 bacteriophage comprising a CRISPR array, (iv) the bacteriophage at least 70%, at least 75%, at least 80%, at least 85%, at least 90%, at least 95%, at least 96%, at least 97%, at least 98%, at least 99%, or 100% identical a M13 bacteriophage comprising a CRISPR array, (v) the bacteriophage at least 70%, at least 75%, at least 80%, at least 85%, at least 90%, at least 95%, at least 96%, at least 97%, at least 98%, at least 99%, or 100% identical to a bacteriophage disclosed herein, (vi) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126317, (vii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126320, (viii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126316, (ix) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126324, and (x) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126315.

In some embodiments, the composition comprises (i) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126324, (ii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126315, and (iii) the bacteriophage at least 70%, at least 75%, at least 80%, at least 85%, at least 90%, at least 95%, at least 96%, at least 97%, at least 98%, at least 99%, or 100% identical to a bacteriophage deposited under ATCC Patent Deposit number PTA-126319.

### Antimicrobial Agents and Peptides

In some embodiments, a bacteriophage disclosed herein is further genetically modified to express an antibacterial peptide, a functional fragment of an antibacterial peptide or a lytic gene. In some embodiments, a bacteriophage disclosed herein express at least one antimicrobial agent or peptide disclosed herein. In some embodiments, a bacteriophage disclosed herein comprises a nucleic acid sequence that encodes an enzybiotic where the protein product of the nucleic acid sequence targets phage resistant bacteria. In some embodiments, the bacteriophage comprises nucleic acids which encode enzymes which assist in breaking down or degrading biofilm matrix. In some embodiments, a bacteriophage disclosed herein comprises nucleic acids encoding Dispersin D aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, xylanase or lyase. In some embodiments, the enzyme is selected from the group consisting of cellulases, such as glycosyl hydroxylase family of cellulases, such as glycosyl hydroxylase 5 family of enzymes also called cellulase A; polyglucosamine (PGA) depolymerases; and colonic acid depolymerases, such as 1,4-L-fucodise hydrolase, colanic acid, depolymerazing alginase, DNase I, or combinations thereof. In some embodiments, a bacteriophage disclosed herein secretes an enzyme disclosed herein.

In some embodiments, an antimicrobial agent or peptide is expressed and/or secreted by a bacteriophage disclosed herein. In some embodiments, a bacteriophage disclosed herein secretes and/or expresses an antibiotic such as ampicillin, penicillin, penicillin derivatives, cephalosporins, monobactams, carbapenems, ofloxacin, ciproflaxacin, levofloxacin, gatifloxacin, norfloxacin, lomefloxacin, trovafloxacin, moxifloxacin, sparfloxacin, gemifloxacin, pazufloxacin or any antibiotic disclosed herein. In some embodiments, a bacteriophage disclosed herein comprises a nucleic acid sequence encoding an antibacterial peptide, expresses an antibacterial peptide, or secretes a peptide that aids or enhances killing of a target bacterium. In some embodiments, a bacteriophage disclosed herein comprises a nucleic acid sequence encoding a peptide, a nucleic acid sequence encoding an antibacterial peptide, expresses an antibacterial peptide, or secretes a peptide that aids or enhances the activity of the first and/or the second Type I CRISPR-Cas system.

### Methods of Use

Disclosed herein, in certain embodiments, are methods of killing a target bacterium comprising introducing into a target bacterium (a) any of the bacteriophages disclosed herein, (b) any of the compositions disclosed herein, or (c) any of the pharmaceutical compositions disclosed herein.

Further disclosed herein, in certain embodiments, are methods of modifying a mixed population of bacterial cells having a first bacterial species that comprises a target nucleotide sequence in the essential gene and a second bacterial species that does not comprise a target nucleotide sequence in the essential gene, the method comprising introducing into the mixed population of bacterial cells (a) any of the bacteriophages disclosed herein, (b) any of the compositions disclosed herein, or (c) any of the pharmaceutical compositions disclosed herein. In some embodiments, (a) any of the bacteriophages disclosed herein, (b) any of the compositions disclosed herein, or (c) any of the pharmaceutical compositions disclosed herein preferentially kill the first bacterial species that comprises the target nucleotide sequence in the essential gene. In some embodiments, (a) any of the bacteriophages disclosed herein, (b) any of the compositions disclosed herein, or (c) any of the pharmaceutical compositions disclosed herein effectively kill the first bacterial species that comprises the target nucleotide sequence in the essential gene compared to the second bacterial species that does not comprise the target nucleotide sequence in the essential gene, thereby modifying the mixed population of bacterial cells. In some embodiments, the first bacterial species that comprises a target nucleotide sequence in the essential gene is a pathogenic bacterial species. In some embodiments, the second bacterial species that does not comprise a target nucleotide sequence in the essential gene is a non-pathogenic bacterial species.

Disclosed herein, in certain embodiments, are methods of selectively killing a pathogenic bacterial species in a mixed population of bacterial cells comprising a pathogenic bacterial species and a non-pathogenic bacterial species, the method comprising introducing into the mixed population of bacterial cells (a) any of the bacteriophages disclosed herein, (b) any of the compositions disclosed herein, or (c) any of the pharmaceutical compositions disclosed herein. In some embodiments, the pathogenic bacterial species comprises a target nucleotide sequence in an essential gene. In some embodiments, the non-pathogenic bacterial species does not comprise a target nucleotide sequence in an essential gene. In some embodiments, (a) any of the bacteriophages disclosed herein, (b) any of the compositions disclosed herein, or (c) any of the pharmaceutical compositions disclosed herein preferentially kill the pathogenic bacterial species. In some embodiments, (a) any of the bacteriophages disclosed herein, (b) any of the compositions disclosed herein, or (c) any of the pharmaceutical compositions disclosed herein effectively kill the pathogenic bacterial species compared to the non-pathogenic bacterial species. Also disclosed herein, in certain embodiments, are methods of treating a disease in an individual in need thereof, the method comprising administering to the individual (a) any of the bacteriophages disclosed herein, (b) any of the compositions disclosed herein, or (c) any of the pharmaceutical compositions disclosed herein.

In some embodiments, the target bacterium is killed solely by lytic activity of the bacteriophage. In some embodiments, the target bacterium is killed solely by activity of the first Type I CRISPR-Cas system. In some embodiments, the target bacterium is killed solely by activity of the second Type I CRISPR-Cas system. In some embodiments, the target bacterium is killed by the processing of the first and/or second CRISPR array by a Type I CRISPR-Cas system to produce a processed crRNA capable of directing CRISPR-Cas based endonuclease activity and/or cleavage at the target nucleotide sequence in the target gene of the bacterium.

In some embodiments, the target bacterium is killed by lytic activity of the bacteriophage in combination with activity of the first Type I CRISPR-Cas system. In some embodiments, the target bacterium is killed by lytic activity of the bacteriophage in combination with activity of the second Type I CRISPR-Cas system. In some embodiments, the target bacterium is killed by the activity of the first Type I CRISPR-Cas system, independently of the lytic activity of the bacteriophage. In some embodiments, the target bacterium is killed by the activity of the second Type I CRISPR-Cas system, independently of the lytic activity of the bacteriophage. In some embodiments, the activity of the first Type I CRISPR-Cas system supplements or enhances the lytic activity of the bacteriophage. In some embodiments, the activity of the second Type I CRISPR-Cas system supplements or enhances the lytic activity of the bacteriophage.

In some embodiments, the lytic activity of the bacteriophage and the activity of the first Type I CRISPR-Cas system is synergistic. In some embodiments, the lytic activity of the bacteriophage and the activity of the second Type I CRISPR-Cas system is synergistic. In some embodiments, the lytic activity of the bacteriophage is modulated by a concentration of the bacteriophage. In some embodiments, the activity of the first Type I CRISPR-Cas system is modulated by a concentration of the bacteriophage. In some embodiments, the activity of the second Type I CRISPR-Cas system, or both, is modulated by a concentration of the bacteriophage.

In some embodiments, the synergistic killing of the bacterium is modulated to favor killing by the lytic activity of the bacteriophage over the activity of the first or second Type I CRISPR-Cas system by increasing the concentration of bacteriophage administered to the bacterium. In some embodiments, the synergistic killing of the bacterium is modulated to disfavor killing by the lytic activity of the bacteriophage over the activity of the first or second Type I CRISPR-Cas system by decreasing the concentration of bacteriophage administered to the bacterium. In some embodiments, at low concentrations, lytic replication allows for amplification and killing of the target bacteria. In some embodiments, at high concentrations, amplification of a phage is not required. In some embodiments, the synergistic killing of the bacterium is modulated to favor killing by the activity of the first or second Type I CRISPR-Cas system over the lytic activity of the bacteriophage by altering the number, the length, the composition, the identity, or any combination thereof, of the spacers so as to increase the lethality of the first and/or the second CRISPR array. In some embodiments, the synergistic killing of the bacterium is modulated to disfavor killing by the activity of the first or second Type I CRISPR-Cas system over the lytic activity of the bacteriophage by altering the number, the length, the composition, the identity, or any combination thereof, of the spacers so as to decrease the lethality of the first and/or the second CRISPR array.

### Administration Routes and Dosage

Dose and duration of the administration of a composition disclosed herein will depend on a variety of factors, including the subject's age, subject's weight, and tolerance of the phage. In some embodiments, a bacteriophage disclosed herein is administered to patients intra-arterially, intravenously, intraurethrally, intramuscularly, orally, subcutaneously, by inhalation, topically, or any combination thereof. In some embodiments, a bacteriophage disclosed herein is administered to patients by oral administration.

In some embodiments, a dose of phage between 10³ and 10²⁰ PFU is given. In some embodiments, a dose of phage between 10³ and 10¹⁰ PFU is given. In some embodiments, a dose of phage between 10⁶ and 10²⁰ PFU is given. In some embodiments, a dose of phage between 10⁶ and 10¹⁰ PFU is given. For example, in some embodiments, the bacteriophage is present in a composition in an amount between 10³ and 10¹¹ PFU. In some embodiments, the bacteriophage is present in a composition in an amount about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², 10²³, 10²⁴ PFU or more. In some embodiments, the bacteriophage is present in a composition in an amount of less than 10¹ PFU. In some embodiments, the bacteriophage is present in a composition in an amount between 10¹ and 10⁸, 10⁴ and 10⁹, 10⁵ and 10¹⁰, or 10⁷ and 10¹¹ PFU.

In some embodiments, a bacteriophage or a mixture is administered to a subject in need thereof 1, 2, 3, 4, 5, 6, 7, 8, 9, 10, 11, 12, 13, 14, 15, 16, 17, 18, 19, 20, 21, 22, 23, or 24 times a day. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, or 21 times a week. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month. In some embodiments, a bacteriophage or a mixture is administered to a subject in need thereof every 2, 4, 6, 8, 10, 12, 14, 18, 20, 22, or 24 hours.

In some embodiments, the compositions (bacteriophage) disclosed herein are administered before, during, or after the occurrence of a disease or condition. In some embodiment, the timing of administering the composition containing the bacteriophage varies. In some embodiments, the pharmaceutical compositions are used as a prophylactic and are administered continuously to subjects with a propensity to conditions or diseases in order to prevent the occurrence of the disease or condition. In some embodiments, pharmaceutical compositions are administered to a subject during or as soon as possible after the onset of the symptoms. In some embodiments, the administration of the compositions is initiated within the first 48 hours of the onset of the symptoms, within the first 24 hours of the onset of the symptoms, within the first 6 hours of the onset of the symptoms, or within 3 hours of the onset of the symptoms. In some embodiments, the initial administration of the composition is via any route practical, such as by any route described herein using any formulation described herein. In some embodiments, the compositions is administered as soon as is practicable after the onset of a disease or condition is detected or suspected, and for a length of time necessary for the treatment of the disease, such as, for example, from about 1 month to about 3 months. In some embodiments, the length of treatment will vary for each subject.

### Bacterial Infections

Disclosed herein, in certain embodiments, are methods of treating bacterial infections. In some embodiments, the bacteriophages disclosed herein treat or prevent diseases or conditions mediated or caused by bacteria as disclosed herein in a human or animal subject. In some embodiments, the bacteriophage disclosed herein treat or prevent diseases or conditions caused or exacerbated by bacteria as disclosed herein in a human or animal subject. Such bacteria are typically in contact with tissue of the subject including: gut, oral cavity, lung, armpit, ocular, vaginal, anal, ear, nose or throat tissue. In some embodiments, a bacterial infection is treated by modulating the activity of the bacteria and/or by directly killing of the bacteria.

In some embodiments, one or more target bacteria present in a bacterial population are pathogenic. In some embodiments, the target bacterium is E. coli. In some embodiments, the E. coli is a multidrug-resistant (MDR) strain. An MDR strain is a bacteria strain that is resistant to at least one antibiotic. In some embodiments, a bacteria strain is resistant to an antibiotic class such as a cephalosporin, a fluoroquinolone, a carbapenem, a colistin, an aminoglycoside, vancomycin, streptomycin, and methicillin. In some embodiments, a bacteria strain is resistant to an antibiotic such as a Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, or any combination thereof. In some embodiments, the E. coli is a extended spectrum beta-lactamase (ESBL) strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the pathogenic bacteria are uropathogenic. In some embodiments, the pathogenic bacterium is uropathogenic E. coli (UPEC). In some embodiments, the pathogenic bacteria are diarrheagenic. In some embodiments, the pathogenic bacteria are diarrheagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacteria are various O-antigen:H-antigen serotype E. coli. In some embodiments, the pathogenic bacteria are entereopathogenic. In some embodiments, the pathogenic bacterium is entereopathogenic E. coli (EPEC).

In some embodiments, the bacteriophages disclosed herein are used to treat an infection, a disease, or a condition, in the gastrointestinal tract of a subject. In some embodiments, the infection or disease is acquired through means including, but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias. In some embodiments, the bacteriophages disclosed herein are used to treat a urinary tract infection (UTI). In some embodiments, the bacteriophages disclosed herein are used to treat an inflammatory disease. In some embodiments, the bacteriophages disclosed herein are used to treat an inflammatory bowel disease (IBD). In some embodiments, the bacteriophages disclosed herein are used to treat Crohn's disease. In some embodiments, the bacteriophages disclosed herein are used to treat Ulcerative colitis.

In some embodiments, the bacteriophages disclosed herein are used to treat an infection, a disease, or a condition, in the urinary tract of a subject. In some embodiments, the bacteriophages are used to modulate and/or kill target bacteria within the urinary tract flora of a subject. In some embodiments, the bacteriophages are used to selectively modulate and/or kill one or more target uropathogenic bacteria from a plurality of bacteria within the urinary tract flora of a subject. In some embodiments, the target bacterium is uropathogenic E. coli (UPEC).

In some embodiments, the bacteriophages disclosed herein are used to treat an infection, a disease, or a condition, on the skin of a subject. In some embodiments, the bacteriophages are used to modulate and/or kill target bacteria on the skin of a subject.

In some embodiments, the bacteriophages disclosed herein are used to treat an infection, a disease, or a condition, on a mucosal membrane of a subject. In some embodiments, the bacteriophages are used to modulate and/or kill target bacteria on the mucosal membrane of a subject. In some embodiments, the bacteriophage treats acne and other related skin infections. In some embodiments, a target bacterium is a multiple drug resistant (MDR) bacteria strain. An MDR strain is a bacteria strain that is resistant to at least one antibiotic. In some embodiments, a bacteria strain is resistant to an antibiotic class such as a cephalosporin, a fluoroquinolone, a carbapenem, a colistin, an aminoglycoside, vancomycin, streptomycin, and methicillin. In some embodiments, a bacteria strain is resistant to an antibiotic such as a Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, or any combination thereof.

In some embodiments, the one or more target bacteria present in the bacterial population form a biofilm. In some embodiments, the biofilm comprises pathogenic bacteria. In some embodiments, the bacteriophage disclosed herein is used to treat a biofilm.

In some embodiments, the methods and compositions disclosed herein are for use in veterinary and medical applications as well as research applications.

### Microbiome

“Microbiome”, “microbiota”, and “microbial habitat” are used interchangeably hereinafter and refer to the ecological community of microorganisms that live on or in a subject's bodily surfaces, cavities, and fluids. Non-limiting examples of habitats of microbiome include: gut, colon, skin, skin surfaces, skin pores, vaginal cavity, umbilical regions, conjunctival regions, intestinal regions, stomach, nasal cavities and passages, gastrointestinal tract, urogenital tracts, saliva, mucus, and feces. In some embodiments, the microbiome comprises microbial material including, but not limited to, bacteria, archaea, protists, fungi, and viruses. In some embodiments, the microbial material comprises a gram-negative bacterium. In some embodiments, the microbial material comprises a gram-positive bacterium.

In some embodiments, the bacteriophages as disclosed herein are used to modulate or kill target bacteria within the microbiome of a subject. In some embodiments, the bacteriophages are used to modulate and/or kill target bacteria within the microbiome by the CRISPR-Cas system, lytic activity, or a combination thereof. In some embodiments, the bacteriophages are used to modulate and/or kill target bacteria within the microbiome of a subject. In some embodiments, the bacteriophages are used to selectively modulate and/or kill one or more target bacteria from a plurality of bacteria within the microbiome of a subject. In some embodiments, the target bacterium is E. coli. In some embodiments, the E. coli is a multidrug-resistant (MDR) strain. In some embodiments, the E. coli is a extended spectrum beta-lactamase (ESBL) strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the pathogenic bacteria are uropathogenic. In some embodiments, the pathogenic bacterium is uropathogenic E. coli (UPEC). In some embodiments, the pathogenic bacteria are diarrheagenic. In some embodiments, the pathogenic bacteria are diarrheagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacteria are various O-antigen:H-antigen serotype E. coli. In some embodiments, the pathogenic bacteria are entereopathogenic. In some embodiments, the pathogenic bacterium is entereopathogenic E. coli (EPEC).

In some embodiments, the bacteriophages are used to modulate or kill target single or plurality of bacteria within the microbiome or gut flora of the gastrointestinal tract of a subject. Modification (e.g., dysbiosis) of the microbiome or gut flora increases the risk for health conditions such as diabetes, mental disorders, ulcerative colitis, colorectal cancer, autoimmune disorders, obesity, diabetes, diseases of the central nervous system and inflammatory bowel disease. An exemplary bacteria associated with diseases and conditions of gastrointestinal tract and are being modulated or killed by the bacteriophages include strains, sub-strains, and enterotypes of E. coli.

In some embodiments, a bacteriophage disclosed herein is administered to a subject to promote a healthy microbiome. In some embodiments, a bacteriophage disclosed herein is administered to a subject to restore a subject's microbiome to a microbiome composition that promotes health. In some embodiments, a composition comprising a bacteriophage disclosed herein comprises a prebiotic, a probiotic, or a third agent. In some embodiment, microbiome related disease or disorder is treated by a bacteriophage disclosed herein.

### Environmental Therapy

In some embodiments, bacteriophages disclosed herein are further used for food and agriculture sanitation (including meats, fruits and vegetable sanitation), hospital sanitation, home sanitation, vehicle and equipment sanitation, industrial sanitation, etc. In some embodiments, bacteriophages disclosed herein are used for the removal of antibiotic-resistant or other undesirable pathogens from medical, veterinary, animal husbandry, or any additional environments bacteria that are passed to humans or animals.

Environmental applications of phage in health care institutions are for equipment such as endoscopes and environments such as ICUs which are potential sources of nosocomial infection due to pathogens that are difficult or impossible to disinfect. In some embodiments, a phage disclosed herein is used to treat equipment or environments inhabited by bacterial genera which become resistant to commonly used disinfectants. In some embodiments, phage compositions disclosed herein are used to disinfect inanimate objects. In some embodiments, an environment disclosed herein is sprayed, painted, or poured onto with aqueous solutions with phage titers. In some embodiment a solution described herein comprises between 10¹-10²⁰ plaque forming units (PFU)/ml. In some embodiments, a bacteriophage disclosed herein is applied by aerosolizing agents that include dry dispersants to facilitate distribution of the bacteriophage into the environment. In some embodiments, objects are immersed in a solution containing bacteriophage disclosed herein.

### Sanitation

In some embodiments, bacteriophages disclosed herein are used as sanitation agents in a variety of fields. Although the terms “phage” or “bacteriophage” may be used, it should be noted that, where appropriate, this term should be broadly construed to include a single bacteriophage, multiple bacteriophages, such as a bacteriophage mixtures and mixtures of a bacteriophage with an agent, such as a disinfectant, a detergent, a surfactant, water, etc.

In some embodiments, bacteriophages are used to sanitize hospital facilities, including operating rooms, patient rooms, waiting rooms, lab rooms, or other miscellaneous hospital equipment. In some embodiments, this equipment includes electrocardiographs, respirators, cardiovascular assist devices, intraaortic balloon pumps, infusion devices, other patient care devices, televisions, monitors, remote controls, telephones, beds, etc. In some situations, the bacteriophage is applied through an aerosol canister. In some embodiments, bacteriophage is applied by wiping the phage on the object with a transfer vehicle.

In some embodiments, a bacteriophage described herein is used in conjunction with patient care devices. In some embodiment, bacteriophage is used in conjunction with a conventional ventilator or respiratory therapy device to clean the internal and external surfaces between patients. Examples of ventilators include devices to support ventilation during surgery, devices to support ventilation of incapacitated patients, and similar equipment. In some embodiments, the conventional therapy includes automatic or motorized devices, or manual bag-type devices such as are commonly found in emergency rooms and ambulances. In some embodiments, respiratory therapy includes inhalers to introduce medications such as bronchodilators as commonly used with chronic obstructive pulmonary disease or asthma, or devices to maintain airway patency such as continuous positive airway pressure devices.

In some embodiment, a bacteriophage described herein is used to cleanse surfaces and treat colonized people in an area where highly-contagious bacterial diseases, such as meningitis or enteric infections are present.

In some embodiments, water supplies are treated with a composition disclosed herein. In some embodiments, bacteriophage disclosed herein is used to treat contaminated water, water found in cisterns, wells, reservoirs, holding tanks, aqueducts, conduits, and similar water distribution devices. In some embodiments, the bacteriophage is applied to industrial holding tanks where water, oil, cooling fluids, and other liquids accumulate in collection pools. In some embodiments, a bacteriophage disclosed herein is periodically introduced to the industrial holding tanks in order to reduce bacterial growth.

In some embodiments, bacteriophages disclosed herein are used to sanitize a living area, such as a house, apartment, condominium, dormitory, or any living area. In some embodiments, the bacteriophage is used to sanitize public areas, such as theaters, concert halls, museums, train stations, airports, pet areas, such as pet beds, or litter boxes. In this capacity, the bacteriophage is dispensed from conventional devices, including pump sprayers, aerosol containers, squirt bottles, pre-moistened towelettes, etc, applied directly to (e.g., sprayed onto) the area to be sanitized, or be transferred to the area via a transfer vehicle, such as a towel, sponge, etc. In some embodiments, a phage disclosed herein is applied to various rooms of a house, including the kitchen, bedrooms, bathrooms, garage, basement, etc. In some embodiments, a phage disclosed herein is in the same manner as conventional cleaners. In some embodiments, the phage is applied in conjunction with (before, after, or simultaneously with) conventional cleaners provided that the conventional cleaner is formulated so as to preserve adequate bacteriophage biologic activity.

In some embodiments, a bacteriophage disclosed herein is added to a component of paper products, either during processing or after completion of processing of the paper products. Paper products to which a bacteriophage disclosed herein is added include, but are not limited to, paper towels, toilet paper, moist paper wipes.

### Food Safety

In some embodiments, a bacteriophage described herein is used in any food product or nutritional supplement, for preventing contamination. Examples for food or pharmaceuticals products are milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.

The broad concept of bacteriophage sanitation is applicable to other agricultural applications and organisms. Produce, including fruits and vegetables, dairy products, and other agricultural products. For example, freshly-cut produce frequently arrive at the processing plant contaminated with pathogenic bacteria. This has led to outbreaks of food-borne illness traceable to produce. In some embodiments, the application of bacteriophage preparations to agricultural produce substantially reduce or eliminate the possibility of food-borne illness through application of a single phage or phage mixture with specificity toward species of bacteria associated with food-borne illness. In some embodiments, bacteriophages are applied at various stages of production and processing to reduce bacterial contamination at that point or to protect against contamination at subsequent points.

In some embodiments, specific bacteriophages are applied to produce in restaurants, grocery stores, produce distribution centers. In some embodiments, bacteriophages disclosed herein are periodically or continuously applied to the fruit and vegetable contents of a salad bar. In some embodiments, the application of bacteriophages to a salad bar or to sanitize the exterior of a food item is a misting or spraying process or a washing process.

In some embodiments, a bacteriophage described herein is used in matrices or support media containing with packaging containing meat, produce, cut fruits and vegetables, and other foodstuffs. In some embodiments, polymers that are suitable for packaging are impregnated with a bacteriophage preparation.

In some embodiments, a bacteriophage described herein is used in farm houses and livestock feed. In some embodiments, on a farm raising livestock, the livestock is provided with bacteriophage in their drinking water, food, or both. In some embodiments, a bacteriophage described herein is sprayed onto the carcasses and used to disinfect the slaughter area.

The use of specific bacteriophages as biocontrol agents on produce provides many advantages. For example, bacteriophages are natural, non-toxic products that will not disturb the ecological balance of the natural microflora in the way the common chemical sanitizers do, but will specifically lyse the targeted food-borne pathogens. Because bacteriophages, unlike chemical sanitizers, are natural products that evolve along with their host bacteria, new phages that are active against recently emerged, resistant bacteria are rapidly identified when required, whereas identification of a new effective sanitizer is a much longer process, several years.

### Pharmaceutical Compositions

Disclosed herein, in certain embodiments, are pharmaceutical compositions comprising (a) the nucleic acid sequences as disclosed herein; and (b) a pharmaceutically acceptable excipient. Also disclosed herein, in certain embodiments, are pharmaceutical compositions comprising (a) the bacteriophages as disclosed herein; and (b) a pharmaceutically acceptable excipient. Further disclosed herein, in certain embodiments, are pharmaceutical compositions comprising (a) the compositions as disclosed herein; and (b) a pharmaceutically acceptable excipient.

In some embodiments, the disclosure provides pharmaceutical compositions and methods of administering the same to treat bacterial, archaeal infections or to disinfect an area. In some embodiments, the pharmaceutical composition comprises any of the reagents discussed above in a pharmaceutically acceptable carrier. In some embodiments, a pharmaceutical composition or method disclosed herein treats urinary tract infections (UTI) and/or inflammatory diseases (e.g. inflammatory bowel disease (IBD)). In some embodiments, a pharmaceutical composition or method disclosed herein treats Crohn's disease. In some embodiments, a pharmaceutical composition or method disclosed herein treats ulcerative colitis.

In some embodiments, compositions disclosed herein comprise medicinal agents, pharmaceutical agents, carriers, adjuvants, dispersing agents, diluents, and the like.

In some embodiments, the bacteriophages disclosed herein are formulated for administration in a pharmaceutical carrier in accordance with suitable methods. In some embodiments, the manufacture of a pharmaceutical composition according to the disclosure, the bacteriophage is admixed with, inter alia, an acceptable carrier. In some embodiments, the carrier is a solid (including a powder) or a liquid, or both, and is preferably formulated as a unit-dose composition. In some embodiments, one or more bacteriophages are incorporated in the compositions disclosed herein, which are prepared by any suitable method of a pharmacy.

In some embodiment, a method of treating subject's in-vivo, comprising administering to a subject a pharmaceutical composition comprising a bacteriophage disclosed herein in a pharmaceutically acceptable carrier, wherein the pharmaceutical composition is administered in a therapeutically effective amount. In some embodiments, the administration of the bacteriophage to a human subject or an animal in need thereof are by any means known in the art.

In some embodiments, bacteriophages disclosed herein are for oral administration. In some embodiments, the bacteriophages are administered in solid dosage forms, such as capsules, tablets, and powders, or in liquid dosage forms, such as elixirs, syrups, and suspensions. In some embodiments, compositions and methods suitable for buccal (sub-lingual) administration include lozenges comprising the bacteriophages in a flavored base, usually sucrose and acacia or tragacanth; and pastilles comprising the bacteriophages in an inert base such as gelatin and glycerin or sucrose and acacia.

In some embodiments, methods and compositions of the present disclosure are suitable for parenteral administration comprising sterile aqueous and non-aqueous injection solutions of the bacteriophage. In some embodiments, these preparations are isotonic with the blood of the intended recipient. In some embodiments, these preparations comprise antioxidants, buffers, bacteriostat, and solutes which render the composition isotonic with the blood of the intended recipient. In some embodiments, aqueous and non-aqueous sterile suspensions include suspending agents and thickening agents. In some embodiments, compositions disclosed herein are presented in unit\dose or multi-dose containers, for example sealed ampoules and vials, and are stored in a freeze-dried (lyophilized) condition requiring only the addition of the sterile liquid carrier, for example, saline or water for injection on immediately prior to use.

In some embodiment, methods and compositions suitable for rectal administration are presented as unit dose suppositories. In some embodiments, these are prepared by admixing the bacteriophage with one or more conventional solid carriers, for example, cocoa butter, and then shaping the resulting mixture. In some embodiments, methods and compositions suitable for topical application to the skin are in the form of an ointment, cream, lotion, paste, gel, spray, aerosol, or oil. In some embodiments, carriers which are used include petroleum jelly, lanoline, polyethylene glycols, alcohols, transdermal enhancers, and combinations of two or more thereof.

In some embodiments, methods and compositions suitable for transdermal administration are presented as discrete patches adapted to remain in intimate contact with the epidermis of the recipient for a prolonged period of time.

In some embodiments, methods and compositions suitable for nasal administration or otherwise administered to the lungs of a subject include any suitable means, e.g., administered by an aerosol suspension of respirable particles comprising the bacteriophage compositions, which the subject inhales. In some embodiments, the respirable particles are liquid or solid. As used herein, “aerosol” includes any gas-borne suspended phase, which is capable of being inhaled into the bronchioles or nasal passages. In some embodiments, aerosols of liquid particles are produced by any suitable means, such as with a pressure-driven aerosol nebulizer or an ultrasonic nebulizer. In some embodiments, aerosols of solid particles comprising the composition is produced with any solid particulate medicament aerosol generator, by techniques known in the pharmaceutical art.

In some embodiment, methods and compositions suitable for administering bacteriophages disclosed herein to a surface of an object or subject includes aqueous solutions. In some embodiments, such aqueous solutions are sprayed onto the surface of an object or subject. In some embodiment, the aqueous solutions are used to irrigate and clean a physical wound of a subject form foreign debris including bacteria.

In some embodiments, the bacteriophages disclosed herein are administered to the subject in a therapeutically effective amount. In some embodiments, at least one bacteriophage composition disclosed herein is formulated as a pharmaceutical formulation. In some embodiments, a pharmaceutical formulation comprises 1, 2, 3, 4, 5, 6, 7, 8, 9, 10, 11, 12, 13, 14, 15, 16, 17, 18, 19, 20 or more bacteriophage disclosed herein. In some instances, a pharmaceutical formulation comprises a bacteriophage described herein and at least one of: an excipient, a diluent, or a carrier.

In some embodiments, a pharmaceutical formulation comprises an excipient. Excipients are described in the Handbook of Pharmaceutical Excipients, American Pharmaceutical Association (1986) and includes but are not limited to solvents, dispersion media, diluents, or other liquid vehicles, dispersion or suspension aids, surface active agents, isotonic agents, thickening or emulsifying agents, preservatives, solid binders, and lubricants.

Non-limiting examples of suitable excipients include but is not limited to a buffering agent, a preservative, a stabilizer, a binder, a compaction agent, a lubricant, a chelator, a dispersion enhancer, a disintegration agent, a flavoring agent, a sweetener, a coloring agent.

In some embodiments, an excipient is a buffering agent. Non-limiting examples of suitable buffering agents include but is not limited to sodium citrate, magnesium carbonate, magnesium bicarbonate, calcium carbonate, and calcium bicarbonate. In some embodiments, a pharmaceutical formulation comprises any one or more buffering agent listed: sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts.

In some embodiments an excipient is a preservative. Non-limiting examples of suitable preservatives include but is not limited to antioxidants, such as alpha-tocopherol and ascorbate, and antimicrobials, such as parabens, chlorobutanol, and phenol. In some embodiments, antioxidants include but not limited to ethylenediamine-N,N,N′,N′-tetraacetic acid (EDTA), citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some embodiments, preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, protease inhibitor, reducing agent, alkylating agent, antimicrobial agent, oxidase inhibitor, or other inhibitor.

In some embodiments, a pharmaceutical formulation comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the binders that are used in a pharmaceutical formulation are selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments, a pharmaceutical formulation comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. In some embodiments, lubricants that are in a pharmaceutical formulation are selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments, an excipient comprises a flavoring agent. In some embodiments, flavoring agents includes natural oils; extracts from plants, leaves, flowers, and fruits; and combinations thereof.

In some embodiments, an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, sylitol, and the like.

In some instances, a pharmaceutical formulation comprises a coloring agent. Non-limiting examples of suitable color agents include food, drug and cosmetic colors (FD&C), drug and cosmetic colors (D&C), and external drug and cosmetic colors (Ext. D&C).

In some embodiments, the pharmaceutical formulation disclosed herein comprises a chelator. In some embodiments, a chelator includes ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA.

In some instances, a pharmaceutical formulation comprises a diluent. Non-limiting examples of diluents include water, glycerol, methanol, ethanol, and other similar biocompatible diluents. In some embodiments, a diluent is an aqueous acid such as acetic acid, citric acid, maleic acid, hydrochloric acid, phosphoric acid, nitric acid, sulfuric acid, or similar.

In some embodiments, a pharmaceutical formulation comprises a surfactant. In some embodiments, surfactants are be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, aminoacids such as L-leucine, sugar esters of fatty acids, glycerides of fatty acids or a combination thereof.

In some instances, a pharmaceutical formulation comprises an additional pharmaceutical agent. In some embodiments, an additional pharmaceutical agent is an antibiotic agent. In some embodiments, an antibiotic agent is of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides or tetracycline.

In some embodiments, an antibiotic agent described herein is an aminoglycoside such as Amikacin, Gentamicin, Kanamycin, Neomycin, Netilmicin, Tobramycin or Paromomycin. In some embodiments, an antibiotic agent described herein is an Ansamycin such as Geldanamycin or Herbimycin

In some embodiments, an antibiotic agent described herein is a carbacephem such as Loracarbef. In some embodiments, an antibiotic agent described herein is a carbapenem such as Ertapenem, Doripenem, Imipenem/Cilastatin or Meropenem.

In some embodiments, an antibiotic agent described herein is a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. In some embodiments, an antibiotic agent is a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime or Ceftobiprole.

In some embodiments, an antibiotic agent described herein is a lincosamide such as Clindamycin and Azithromycin, or a macrolide such as Azithromycin, Clarithromycin, Dirithromycin, Erythromycin, Roxithromycin, Troleandomycin, Telithromycin and Spectinomycin.

In some embodiments, an antibiotic agent described herein is a monobactams such as Aztreonam, or a nitrofuran such as Furazolidone or Nitrofurantoin.

In some embodiments, an antibiotic agent described herein is a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

In some embodiments, an antibiotic agent described herein is a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, or Trimethoprim-Sulfamethoxazole (Co-trimoxazole) (TMP-SMX).

In some embodiments, an antibiotic agent described herein is a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

In some embodiments, an antibiotic agent described herein is a polypeptide such as Bacitracin, Colistin or Polymyxin B.

In some embodiments, an antibiotic agent described herein is a tetracycline such as Demeclocycline, Doxycycline, Minocycline or Oxytetracycline.

### Numbered Embodiments

Numbered embodiment 1 comprises a nucleic acid sequence comprising (a) a first CRISPR array designed to be operable with a first Type I CRISPR-Cas system, and (b) a second CRISPR array designed to be operable with a second Type I CRISPR-Cas system, wherein the first Type I CRISPR-Cas system and the second Type I CRISPR-Cas system are different Type I CRISPR-Cas systems. Numbered embodiment 2 comprises the nucleic acid sequence of embodiment 1, wherein the first CRISPR array comprises a first spacer sequence and the second CRISPR array comprises a second spacer sequence. Numbered embodiment 3 comprises the nucleic acid sequence of any one of embodiments 1-2, wherein the first CRISPR array and the second CRISPR array further comprises at least one repeat sequence. Numbered embodiment 4 comprises the nucleic acid sequence of embodiments 1-3, wherein the at least one repeat sequence is operably linked to the first spacer sequence at either its 5′ end or its 3′ end and/or the second spacer sequence at either its 5′ end or its 3′ end. Numbered embodiment 5 comprises the nucleic acid sequence of any one of embodiments 1-4, wherein the first and/or second spacer sequence is complementary to a target nucleotide sequence of an essential gene in a target bacterium. Numbered embodiment 6 comprises the nucleic acid sequence of embodiments 1-5, wherein the target nucleotide sequence comprises all or a part of a promoter sequence of the essential gene. Numbered embodiment 7 comprises the nucleic acid sequence of embodiments 1-6, wherein the target nucleotide sequence comprises all or a part of a nucleotide sequence located on a coding strand of a transcribed region of the essential gene. Numbered embodiment 8 comprises the nucleic acid sequence of any one of embodiments 1-7, wherein the essential gene is ftsA. Numbered embodiment 9 comprises the nucleic acid sequence of any one of embodiments 1-8, wherein the first and/or second spacer sequence is complementary to a target nucleotide sequence in a non-essential gene. Numbered embodiment 10 comprises the nucleic acid sequence of any one of embodiments 1-9, wherein the first and/or second spacer is completely to a target nucleic acid sequence in a noncoding sequence. Numbered embodiment 11 comprises the nucleic acid sequence of any one of embodiments 1-10, wherein the first CRISPR array and the second CRISPR array are on same nucleic acid sequence. Numbered embodiment 12 comprises the nucleic acid sequence of any one of embodiments 1-11, wherein the nucleic acid sequence further comprises a leuO coding sequence. Numbered embodiment 13 comprises the nucleic acid sequence of any one of embodiments 1-12, wherein the nucleic acid sequence further comprises a leader sequence. Numbered embodiment 14 comprises the nucleic acid sequence of any one of embodiments 1-13, wherein the nucleic acid sequence further comprises a promoter sequence. Numbered embodiment 15 comprises the nucleic acid sequence of any one of embodiments 1-14, wherein the nucleic acid sequence is at least 80%, at least 90%, at least 95%, at least 99%, or 100% identical to a nucleic acid sequence set forth as SEQ ID NO: 1. Numbered embodiment 16 comprises the nucleic acid sequence of any one of embodiments 1-15, wherein the first Type I CRISPR-Cas system is a Type I-A system, Type I-B system, Type I-C system, Type I-D system, Type I-E system, or Type I-F system. Numbered embodiment 17 comprises the nucleic acid sequence of any one of embodiments 1-16, wherein the first Type I CRISPR-Cas system is a Type I-E system. Numbered embodiment 18 comprises the nucleic acid sequence of any one of embodiments 1-17, wherein the second Type I CRISPR-Cas system is a Type I-A system, Type I-B system, Type I-C system, Type I-D system, Type I-E system, or Type I-F system. Numbered embodiment 19 comprises the nucleic acid sequence of any one of embodiments 1-18, wherein the second Type I CRISPR-Cas system is a Type I-F system. Numbered embodiment 20 comprises the nucleic acid sequence of any one of embodiments 1-19, wherein the first Type I CRISPR-Cas system, the second Type I CRISPR-Cas system, or both are endogenous to the target bacterium. Numbered embodiment 21 comprises the nucleic acid sequence of any one of embodiments 1-20, wherein the first Type I CRISPR-Cas system, the second Type I CRISPR-Cas system, or both are exogenous to the target bacterium. Numbered embodiment 22 comprises the nucleic acid sequence of any one of embodiments 1-21, wherein the target bacterium is E. coli. Numbered embodiment 23 comprises the nucleic acid sequence of embodiment 1-22, wherein the E. coli is a multidrug-resistant (MDR) strain. Numbered embodiment 24 comprises the nucleic acid sequence of embodiment 1-23, wherein the E. coli is an extended spectrum beta-lactamase (ESBL) strain. Numbered embodiment 25 comprises the nucleic acid sequence of embodiment 1-24, wherein the E. coli is a carbapenem-resistant strain. Numbered embodiment 26 comprises the nucleic acid sequence of embodiments 1-25, wherein the E. coli is a non-multidrug-resistant (non-MDR) strain. Numbered embodiment 27 comprises the nucleic acid sequence of embodiments 1-26, wherein the E. coli is a non-carbapenem-resistant strain. Numbered embodiment 28 comprises the nucleic acid sequence of any one of embodiments 1-27, wherein the E. coli causes urinary tract infection. Numbered embodiment 29 comprises the nucleic acid sequence of any one of embodiments 1-28, wherein the E. coli causes inflammatory bowel disease (IBD). Numbered embodiment 30 comprises a bacteriophage comprising a nucleic acid sequence comprising (a) a first CRISPR array designed to be operable with a first Type I CRISPR-Cas system, and (b) a second CRISPR array designed to be operable with a second Type I CRISPR-Cas system, wherein the first Type I CRISPR-Cas system and the second Type I CRISPR-Cas system are different Type I CRISPR-Cas systems. Numbered embodiment 31 comprises the bacteriophage of embodiments 1-30, wherein the first CRISPR array comprises a first spacer sequence and the second CRISPR array comprises a second spacer sequence. Numbered embodiment 32 comprises the bacteriophage of any one of embodiments 1-31, wherein the first CRISPR array and the second CRISPR array further comprises at least one repeat sequence. Numbered embodiment 33 comprises the bacteriophage of embodiments 1-32, wherein the at least one repeat sequence is operably linked to the first spacer sequence at either its 5′ end or its 3′ end and/or the second spacer sequence at either its 5′ end or its 3′ end. Numbered embodiment 34 comprises the bacteriophage of any one of embodiments 1-33, wherein the first spacer sequence and/or the second spacer sequence is complementary to a target nucleotide sequence of an essential gene in a target bacterium. Numbered embodiment 35 comprises the bacteriophage of embodiment 1-34, wherein the target nucleotide sequence comprises all or a part of a promoter sequence of the essential gene. Numbered embodiment 36 comprises the bacteriophage of embodiment 1-35, wherein the target nucleotide sequence comprises all or a part of a nucleotide sequence located on a coding strand of a transcribed region of the essential gene. Numbered embodiment 37 comprises the bacteriophage of any one of embodiments 1-36, wherein the essential gene is ftsA. Numbered embodiment 38 comprises the bacteriophage of any one of embodiments 1-37, wherein the first CRISPR array and the second CRISPR array are on same nucleic acid sequence. Numbered embodiment 39 comprises the bacteriophage of any one of embodiments 1-38, wherein the nucleic acid sequence further comprises a leuO coding sequence. Numbered embodiment 40 comprises the bacteriophage of any one of embodiments 1-39, wherein the nucleic acid sequence further comprises a leader sequence. Numbered embodiment 41 comprises the bacteriophage of any one of embodiments 1-40, wherein the nucleic acid sequence further comprises a promoter sequence. Numbered embodiment 42 comprises the bacteriophage of any one of embodiments 1-41, wherein the nucleic acid sequence is at least 80%, at least 90%, at least 95%, at least 99%, or 100% identical to a nucleic acid sequence set forth as SEQ ID NO: 1. Numbered embodiment 43 comprises the bacteriophage of any one of embodiments 1-42, wherein the first Type I CRISPR-Cas system is a Type I-A system, Type I-B system, Type I-C system, Type I-D system, Type I-E system, or Type I-F system. Numbered embodiment 44 comprises the bacteriophage of any one of embodiments 1-43, wherein the first Type I CRISPR-Cas system is a Type I-E system. Numbered embodiment 45 comprises the bacteriophage of any one of embodiments 1-44, wherein the second Type I CRISPR-Cas system is a Type I-A system, Type I-B system, Type I-C system, Type I-D system, Type I-E system, or Type I-F system. Numbered embodiment 46 comprises the bacteriophage of any one of embodiments 1-45, wherein the second Type I CRISPR-Cas system is a Type I-F system. Numbered embodiment 47 comprises the bacteriophage of any one of embodiments 1-46, wherein the first Type I CRISPR-Cas system, the second Type I CRISPR-Cas system, or both are endogenous to the target bacterium. Numbered embodiment 48 comprises the bacteriophage of any one of embodiments 1-47, wherein the first Type I CRISPR-Cas system, the second Type I CRISPR-Cas system, or both are exogenous to the target bacterium. Numbered embodiment 49 comprises the bacteriophage of any one of embodiments 1-48, wherein the target bacterium is killed solely by lytic activity of the bacteriophage. Numbered embodiment 50 comprises the bacteriophage of any one of embodiments 1-49, wherein the target bacterium is killed solely by activity of the first Type I CRISPR-Cas system or the second Type I CRISPR-Cas system. Numbered embodiment 51 comprises the bacteriophage of any one of embodiments 1-50, wherein the target bacterium is killed by lytic activity of the bacteriophage in combination with activity of the first Type I CRISPR-Cas system or the second Type I CRISPR-Cas system. Numbered embodiment 52 comprises the bacteriophage of any one of embodiments 1-51, wherein the target bacterium is killed by the activity of the first Type I CRISPR-Cas system or the second Type I CRISPR-Cas system, independently of the lytic activity of the bacteriophage. Numbered embodiment 53 comprises the bacteriophage of any one of embodiments 1-52, wherein the activity of the first Type I CRISPR-Cas system or the second Type I CRISPR-Cas system supplements or enhances the lytic activity of the bacteriophage. Numbered embodiment 54 comprises the bacteriophage of any one of embodiments 1-53, wherein the lytic activity of the bacteriophage and the activity of the first Type I CRISPR-Cas system or the second Type I CRISPR-Cas system are synergistic. Numbered embodiment 55 comprises the bacteriophage of any one of embodiments 1-54, wherein the lytic activity of the bacteriophage, the activity of the first Type I CRISPR-Cas system or the second Type I CRISPR-Cas system, or both, is modulated by a concentration of the bacteriophage. Numbered embodiment 56 comprises the bacteriophage of any one of embodiments 1-55, wherein the target bacterium is E. coli. Numbered embodiment 57 comprises the bacteriophage of embodiments 1-56, wherein the E. coli is a multidrug-resistant (MDR) strain. Numbered embodiment 58 comprises the bacteriophage of embodiments 1-57, wherein the E. coli is an extended spectrum beta-lactamase (ESBL) strain. Numbered embodiment 59 comprises the bacteriophage of embodiments 1-58, wherein the E. coli is a carbapenem-resistant strain. Numbered embodiment 60 comprises the bacteriophage of embodiments 1-59, wherein the E. coli is a non-multidrug-resistant (non-MDR) strain. Numbered embodiment 61 comprises the bacteriophage of embodiments 1-60, wherein the E. coli is a non-carbapenem-resistant strain. Numbered embodiment 62 comprises the bacteriophage of any one of embodiments 1-61, wherein the E. coli causes urinary tract infection. Numbered embodiment 63 comprises the bacteriophage of any one of embodiments 1-62, wherein the E. coli causes inflammatory bowel disease (IBD). Numbered embodiment 64 comprises the bacteriophage of any one of embodiments 1-63, wherein the bacteriophage is an obligate lytic bacteriophage. Numbered embodiment 65 comprises the bacteriophage of any one of embodiments 1-64, wherein the bacteriophage is a temperate bacteriophage with a lysogeny gene removed, replaced, or inactivated, thereby rendering the bacteriophage lytic. Numbered embodiment 66 comprises the bacteriophage of any one of embodiments 1-65, wherein the bacteriophage is PTA-126317, PTA-126320, PTA-126316, PTA-126324, PTA-126315, or PTA-126319. Numbered embodiment 67 comprises the bacteriophage of any one of embodiments 1-66, wherein the nucleic acid sequence is inserted into a non-essential bacteriophage gene. Numbered embodiment 68 comprises the bacteriophage of any one of embodiments 1-67, wherein the bacteriophage is at least 80%, at least 90%, at least 95%, at least 99%, or 100% identical to a bacteriophage deposited under ATCC Patent Deposit number PTA-126317. Numbered embodiment 69 comprises the bacteriophage of any one of embodiments 1-68, wherein the bacteriophage is at least 80%, at least 90%, at least 95%, at least 99%, or 100% identical to a bacteriophage deposited under ATCC Patent Deposit number PTA-126320. Numbered embodiment 70 comprises the bacteriophage of any one of embodiments 1-69, wherein the bacteriophage is at least 80%, at least 90%, at least 95%, at least 99%, or 100% identical to a bacteriophage deposited under ATCC Patent Deposit number PTA-126316. Numbered embodiment 71 comprises the bacteriophage of any one of embodiments 1-70, wherein the bacteriophage is at least 80%, at least 90%, at least 95%, at least 99%, or 100% identical to a bacteriophage deposited under ATCC Patent Deposit number PTA-126324. Numbered embodiment 72 comprises the bacteriophage of any one of embodiments 1-71, the bacteriophage is at least 80%, at least 90%, at least 95%, at least 99%, or 100% identical to a bacteriophage deposited under ATCC Patent Deposit number PTA-126315. Numbered embodiment 73 comprises the bacteriophage of any one of embodiments 1-72, wherein the bacteriophage is at least 80%, at least 90%, at least 95%, at least 99%, or 100% identical to a bacteriophage deposited under ATCC Patent Deposit number PTA-126319. Numbered embodiment 74 comprises a PTA-126317 bacteriophage comprising a nucleic acid sequence comprising (a) a first CRISPR array designed to be operable with a first Type I CRISPR-Cas system, or (b) a leuO coding sequence. Numbered embodiment 75 comprises a PTA-126320 bacteriophage comprising a nucleic acid sequence comprising (a) a first CRISPR array designed to be operable with a first Type I CRISPR-Cas system, or (b) a leuO coding sequence. Numbered embodiment 76 comprises a PTA-126316 bacteriophage comprising a nucleic acid sequence comprising (a) a first CRISPR array designed to be operable with a first Type I CRISPR-Cas system, or (b) a leuO coding sequence. Numbered embodiment 77 comprises a PTA-126324 bacteriophage comprising a nucleic acid sequence comprising (a) a first CRISPR array designed to be operable with a first Type I CRISPR-Cas system, or (b) a leuO coding sequence. Numbered embodiment 78 comprises a PTA-126315 bacteriophage comprising a nucleic acid sequence comprising (a) a first CRISPR array designed to be operable with a first Type I CRISPR-Cas system, or (b) a leuO coding sequence. Numbered embodiment 79 comprises a PTA-126319 bacteriophage comprising a nucleic acid sequence comprising (a) a first CRISPR array designed to be operable with a first Type I CRISPR-Cas system, or (b) a leuO coding sequence. Numbered embodiment 80 comprises the bacteriophage of any one of embodiments 1-79, wherein the nucleic acid sequence comprises (a) a first CRISPR array designed to be operable with a first Type I CRISPR-Cas system. Numbered embodiment 81 comprises the bacteriophage of any one of embodiments 1-80, wherein the nucleic acid sequence comprises (b) a leuO coding sequence. Numbered embodiment 82 comprises the bacteriophage of any one of embodiments 1-81, wherein the nucleic acid sequence comprises (a) a first CRISPR array designed to be operable with a first Type I CRISPR-Cas system, and (b) a leuO coding sequence. Numbered embodiment 83 comprises the bacteriophage of any one of embodiments 1-82, wherein the nucleic acid sequence further comprises (c) a second CRISPR array designed to be operable with a second Type I CRISPR-Cas system. Numbered embodiment 84 comprises the bacteriophage of embodiment 1-83, wherein the first Type I CRISPR-Cas system and the second Type I CRISPR-Cas system are different Type I CRISPR-Cas systems. Numbered embodiment 85 comprises the bacteriophage of any one of embodiments 1-84, wherein the first CRISPR array comprises first spacer sequence and the second CRISPR array comprises a second spacer sequence. Numbered embodiment 86 comprises the bacteriophage of any one of embodiments 1-85, wherein the first CRISPR array and the second CRISPR array further comprises at least one repeat sequence. Numbered embodiment 87 comprises the bacteriophage of embodiments 1-86, wherein the at least one repeat sequence is operably linked to the first spacer sequence at either its 5′ end or its 3′ end and/or the second spacer sequence at either its 5′ end or its 3′ end. Numbered embodiment 88 comprises the bacteriophage of any one of embodiments 1-87, wherein the first spacer sequence and/or the second spacer sequence is complementary to a target nucleotide sequence of an essential gene in a target bacterium. Numbered embodiment 89 comprises the bacteriophage of embodiments 1-88, wherein the target nucleotide sequence comprises all or a part of a promoter sequence of the essential gene. Numbered embodiment 90 comprises the bacteriophage of embodiments 1-89, wherein the target nucleotide sequence comprises all or a part of a nucleotide sequence located on a coding strand of a transcribed region of the essential gene. Numbered embodiment 91 comprises the bacteriophage of any one of embodiments 1-90, wherein the essential gene is ftsA. Numbered embodiment 92 comprises the bacteriophage of any one of embodiments 1-91, wherein the first CRISPR array and the second CRISPR array are on same nucleic acid sequence. Numbered embodiment 93 comprises the bacteriophage of any one of embodiments 1-92, wherein the nucleic acid sequence further comprises a leader sequence. Numbered embodiment 94 comprises the bacteriophage of any one of embodiments 1-93, wherein the nucleic acid sequence further comprises a promoter sequence. Numbered embodiment 95 comprises the bacteriophage of any one of embodiments 1-94, wherein the nucleic acid sequence is at least 80%, at least 90%, at least 95%, at least 99%, or 100% identical to a nucleic acid sequence set forth as SEQ ID NO: 1. Numbered embodiment 96 comprises the bacteriophage of any one of embodiments 1-95, wherein the first Type I CRISPR-Cas system is a Type I-A system, Type I-B system, Type I-C system, Type I-D system, Type I-E system, or Type I-F system. Numbered embodiment 97 comprises the bacteriophage of any one of embodiments 1-96, wherein the first Type I CRISPR-Cas system is a Type I-E system. Numbered embodiment 98 comprises the bacteriophage of any one of embodiments 1-97, wherein the second Type I CRISPR-Cas system is a Type I-A system, Type I-B system, Type I-C system, Type I-D system, Type I-E system, or Type I-F system. Numbered embodiment 99 comprises the bacteriophage of any one of embodiments 1-98, wherein the second Type I CRISPR-Cas system is a Type I-F system. Numbered embodiment 100 comprises the bacteriophage of any one of embodiments 1-99, wherein the first Type I CRISPR-Cas system, the second Type I CRISPR-Cas system, or both are endogenous to the target bacterium. Numbered embodiment 101 comprises the bacteriophage of any one of embodiments 1-100, wherein the first Type I CRISPR-Cas system, the second Type I CRISPR-Cas system, or both are exogenous to the target bacterium. Numbered embodiment 102 comprises the bacteriophage of any one of embodiments 1-101, wherein the target bacterium is killed solely by lytic activity of the bacteriophage. Numbered embodiment 103 comprises the bacteriophage of any one of embodiments 1-102, wherein the target bacterium is killed solely by activity of the first Type I CRISPR-Cas system or the second Type I CRISPR-Cas system. Numbered embodiment 104 comprises the bacteriophage of any one of embodiments 1-103, wherein the target bacterium is killed by lytic activity of the bacteriophage in combination with activity of the first Type I CRISPR-Cas system or the second Type I CRISPR-Cas system. Numbered embodiment 105 comprises the bacteriophage of any one of embodiments 1-104, wherein the target bacterium is killed by the activity of the first Type I CRISPR-Cas system or the second Type I CRISPR-Cas system, independently of the lytic activity of the bacteriophage. Numbered embodiment 106 comprises the bacteriophage of any one of embodiments 1-105, wherein the activity of the first Type I CRISPR-Cas system or the second Type I CRISPR-Cas system supplements or enhances the lytic activity of the bacteriophage. Numbered embodiment 107 comprises the bacteriophage of any one of embodiments 1-106, wherein the lytic activity of the bacteriophage and the activity of the first Type I CRISPR-Cas system or the second Type I CRISPR-Cas system are synergistic. Numbered embodiment 108 comprises the bacteriophage of any one of embodiments 1-107, wherein the lytic activity of the bacteriophage, the activity of the first Type I CRISPR-Cas system or the second Type I CRISPR-Cas system, or both, is modulated by a concentration of the bacteriophage. Numbered embodiment 109 comprises the bacteriophage of any one of embodiments 1-108, wherein the target bacterium is E. coli. Numbered embodiment 110 comprises the bacteriophage of embodiments 1-109, wherein the E. coli is a multidrug-resistant (MDR) strain. Numbered embodiment 111 comprises the bacteriophage of embodiments 1-110, wherein the E. coli is an extended spectrum beta-lactamase (ESBL) strain. Numbered embodiment 112 comprises the bacteriophage of embodiments 1-111, wherein the E. coli is a carbapenem-resistant strain. Numbered embodiment 113 comprises the bacteriophage of embodiments 1-112, wherein the E. coli is a non-multidrug-resistant (non-MDR) strain. Numbered embodiment 114 comprises the bacteriophage of embodiments 1-113, wherein the E. coli is a non-carbapenem-resistant strain. Numbered embodiment 115 comprises the bacteriophage of any one of embodiments 1-114, wherein the E. coli causes urinary tract infection. Numbered embodiment 116 comprises the bacteriophage of any one of embodiments 1-115, wherein the E. coli causes inflammatory bowel disease (IBD). Numbered embodiment 117 comprises the bacteriophage of any one of embodiments 1-116, wherein the bacteriophage is an obligate lytic bacteriophage. Numbered embodiment 118 comprises the bacteriophage of any one of embodiments 1-117, wherein the nucleic acid sequence is inserted into a non-essential bacteriophage gene. Numbered embodiment 119 comprises the bacteriophage of any one of embodiments 1-118, wherein the bacteriophage is at least 80%, at least 90%, at least 95%, at least 99%, or 100% identical to a bacteriophage deposited under ATCC Patent Deposit number PTA-126317. Numbered embodiment 120 comprises the bacteriophage of any one of embodiments 1-119, wherein the bacteriophage is at least 80%, at least 90%, at least 95%, at least 99%, or 100% identical to a bacteriophage deposited under ATCC Patent Deposit number PTA-126320. Numbered embodiment 121 comprises the bacteriophage of any one of embodiments 1-120, wherein the bacteriophage is at least 80%, at least 90%, at least 95%, at least 99%, or 100% identical to a bacteriophage deposited under ATCC Patent Deposit number PTA-126316. Numbered embodiment 122 comprises the bacteriophage of any one of embodiments 1-121, wherein the bacteriophage is at least 80%, at least 90%, at least 95%, at least 99%, or 100% identical to a bacteriophage deposited under ATCC Patent Deposit number PTA-126324. Numbered embodiment 123 comprises the bacteriophage of any one of embodiments 1-122, wherein the bacteriophage is at least 80%, at least 90%, at least 95%, at least 99%, or 100% identical to a bacteriophage deposited under ATCC Patent Deposit number PTA-126315. Numbered embodiment 124 comprises the bacteriophage of any one of embodiments 1-123, wherein the bacteriophage is at least 80%, at least 90%, at least 95%, at least 99%, or 100% identical to a bacteriophage deposited under ATCC Patent Deposit number PTA-126319. Numbered embodiment 125 comprises a composition comprising: at least two bacteriophages selected from a list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126316, (iv) a bacteriophage comprising at least 80% identity to a bacteriophage deposited under ATCC Patent Deposit number PTA-126324, (v) a bacteriophage comprising at least 80% identity to a bacteriophage deposited under ATCC Patent Deposit number PTA-126315, and (vi) a bacteriophage comprising at least 80% identity to a bacteriophage deposited under ATCC Patent Deposit number PTA-126319. Numbered embodiment 126 comprises a composition comprising: at least three bacteriophages selected from a list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126316, (iv) a bacteriophage comprising at least 80% identity to a bacteriophage deposited under ATCC Patent Deposit number PTA-126324, (v) a bacteriophage comprising at least 80% identity to a bacteriophage deposited under ATCC Patent Deposit number PTA-126315, and (vi) a bacteriophage comprising at least 80% identity to a bacteriophage deposited under ATCC Patent Deposit number PTA-126319. Numbered embodiment 127 comprises a composition comprising: at least six bacteriophages selected from a list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126316, (iv) a bacteriophage comprising at least 80% identity to a bacteriophage deposited under ATCC Patent Deposit number PTA-126324, (v) a bacteriophage comprising at least 80% identity to a bacteriophage deposited under ATCC Patent Deposit number PTA-126315, and (vi) a bacteriophage comprising at least 80% identity to a bacteriophage deposited under ATCC Patent Deposit number PTA-126319. Numbered embodiment 128 comprises a composition comprising: (a) a bacteriophage comprising at least 80% identity to a bacteriophage deposited under ATCC Patent Deposit number PTA-126324; (b) a bacteriophage comprising at least 80% identity to a bacteriophage deposited under ATCC Patent Deposit number PTA-126315; and (c) a bacteriophage comprising at least 80% identity to a bacteriophage deposited under ATCC Patent Deposit number PTA-126319. Numbered embodiment 129 comprises the composition of embodiments 1-128 wherein (a) the bacteriophage comprises at least 90%, at least 95%, at least 99%, or 100% identity to a bacteriophage deposited under ATCC Patent Deposit number PTA-126324. Numbered embodiment 130 comprises the composition of any one of embodiments 1-129, wherein (b) the bacteriophage comprises at least 90%, at least 95%, at least 99%, or 100% identity to a bacteriophage deposited under ATCC Patent Deposit number PTA-126315. Numbered embodiment 131 comprises the composition of any one of embodiments 1-130, wherein (c) the bacteriophage comprises at least 90%, at least 95%, at least 99%, or 100% identity to a bacteriophage deposited under ATCC Patent Deposit number PTA-126319. Numbered embodiment 132 comprises a composition comprising: (a) a bacteriophage comprising at least 80% identity to a bacteriophage deposited under ATCC Patent Deposit number PTA-126317, and (b) at least one more bacteriophage selected from the list consisting of: (i) a bacteriophage comprising at least 80% identity to a bacteriophage deposited under ATCC Patent Deposit number PTA-126320, (ii) a bacteriophage comprising at least 80% identity to a bacteriophage deposited under ATCC Patent Deposit number PTA-126316, (iii) a bacteriophage comprising at least 80% identity to a bacteriophage deposited under ATCC Patent Deposit number PTA-126324, (iv) a bacteriophage comprising at least 80% identity to a bacteriophage deposited under ATCC Patent Deposit number PTA-126315, and (v) a bacteriophage comprising at least 80% identity to a bacteriophage deposited under ATCC Patent Deposit number PTA-126319. Numbered embodiment 133 comprises a composition comprising: (a) a bacteriophage comprising at least 80% identity to a bacteriophage deposited under ATCC Patent Deposit number PTA-126320, and (b) at least one more bacteriophage selected from the list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126316, (iii) a bacteriophage comprising at least 80% identity to a bacteriophage deposited under ATCC Patent Deposit number PTA-126324, (iv) a bacteriophage comprising at least 80% identity to a bacteriophage deposited under ATCC Patent Deposit number PTA-126315, and (v) a bacteriophage comprising at least 80% identity to a bacteriophage deposited under ATCC Patent Deposit number PTA-126319. Numbered embodiment 134 comprises a composition comprising: (a) a bacteriophage comprising at least 80% identity to a bacteriophage deposited under ATCC Patent Deposit number PTA-126316, and (b) at least one more bacteriophage selected from the list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126324, (iv) a bacteriophage comprising at least 80% identity to a bacteriophage deposited under ATCC Patent Deposit number PTA-126315, and (v) a bacteriophage comprising at least 80% identity to a bacteriophage deposited under ATCC Patent Deposit number PTA-126319. Numbered embodiment 135 comprises a composition comprising: (a) a bacteriophage comprising at least 80% identity to a bacteriophage deposited under ATCC Patent Deposit number PTA-126324, and (b) at least one more bacteriophage selected from the list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126316, (iv) a bacteriophage comprising at least 80% identity to a bacteriophage deposited under ATCC Patent Deposit number PTA-126315, and (v) a bacteriophage comprising at least 80% identity to a bacteriophage deposited under ATCC Patent Deposit number PTA-126319. Numbered embodiment 136 comprises a composition comprising: (a) a bacteriophage comprising at least 80% identity to a bacteriophage deposited under ATCC Patent Deposit number PTA-126315, and (b) at least one more bacteriophage selected from the list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126316, (iv) a bacteriophage comprising at least 80% identity to a bacteriophage deposited under ATCC Patent Deposit number PTA-126324, (v) a bacteriophage comprising at least 80% identity to a bacteriophage deposited under ATCC Patent Deposit number PTA-126319. Numbered embodiment 137 comprises a composition comprising: (a) a bacteriophage comprising at least 80% identity to a bacteriophage deposited under ATCC Patent Deposit number PTA-126319, and (b) at least one more bacteriophage selected from the list consisting of: (i) a bacteriophage comprising at least 80% identity to a bacteriophage deposited under ATCC Patent Deposit number PTA-126317, (ii) a bacteriophage comprising at least 80% identity to a bacteriophage deposited under ATCC Patent Deposit number PTA-126320, (iii) a bacteriophage comprising at least 80% identity to a bacteriophage deposited under ATCC Patent Deposit number PTA-126316, (iv) a bacteriophage comprising at least 80% identity to a bacteriophage deposited under ATCC Patent Deposit number PTA-126324, and (v) a bacteriophage comprising at least 80% identity to a bacteriophage deposited under ATCC Patent Deposit number PTA-126315. Numbered embodiment 138 comprises a pharmaceutical composition comprising: (a) (i) the nucleic acid sequence of embodiments 1-137, (ii) the bacteriophage of any one of embodiments 1-137, or (iii) the composition of any one of embodiments 1-137; and (b) a pharmaceutically acceptable excipient. Numbered embodiment 139 comprises the pharmaceutical composition of embodiment 138, wherein the pharmaceutical composition is in the form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof. Numbered embodiment 140 comprises a method of killing a target bacterium comprising introducing into a target bacterium (a) the bacteriophage of any one of embodiments 1-139, (b) the composition of any one of embodiments 1-139, or (c) the pharmaceutical composition of any one of embodiments 1-139. Numbered embodiment 141 comprises a method modifying a mixed population of bacterial cells having a first bacterial species that comprises a target nucleotide sequence in the essential gene and a second bacterial species that does not comprise a target nucleotide sequence in the essential gene, the method comprising introducing into the mixed population of bacterial cells (a) the bacteriophage of any one of embodiments 1-139, (b) the composition of any one of embodiments 1-139, or (c) the pharmaceutical composition of any one of embodiments 1-139. Numbered embodiment 142 comprises a method of treating a disease in an individual in need thereof, the method comprising administering to the individual (a) the bacteriophage of any one of embodiments 1-139, (b) the composition of any one of embodiments 1-139, or (c) the pharmaceutical composition of any one of embodiments 1-139. Numbered embodiment 143 comprises the method of embodiments 1-142, wherein the disease is a bacterial infection. Numbered embodiment 144 comprises the method of embodiments 1-143, wherein the disease is a urinary tract infection (UTI). Numbered embodiment 145 comprises the method of embodiments 1-144, wherein the disease is inflammatory bowel disease (IBD). Numbered embodiment 146 comprises the method of any one of embodiments 1-145, wherein the individual is a mammal. Numbered embodiment 147 comprises the method of any one of embodiments 1-146, wherein the administering is intra-arterial, intravenous, intraurethral, intramuscular, oral, subcutaneous, inhalation, or any combination thereof. Numbered embodiment 148 comprises the method of any one of embodiments 1-147, wherein (a) the bacteriophage, (b) the composition, or (c) the pharmaceutical composition is administered at a dose of phage between 10⁶ and 10¹⁰ PFU. Numbered embodiment 149 comprises the method of any one of embodiments 1-148, wherein (a) the bacteriophage, (b) the composition, or (c) the pharmaceutical composition is administered 1, 2, 3, 4, or 5 times daily. Numbered embodiment 150 comprises the method of any one of embodiments 1-149, wherein (a) the bacteriophage, (b) the composition, or (c) the pharmaceutical composition is administered 2 times daily. Numbered embodiment 151 comprises the method of any one of embodiments 1-150, wherein (a) the bacteriophage, (b) the composition, or (c) the pharmaceutical composition is administered every 12 hours. Numbered embodiment 152 comprises a method of treating a urinary tract infection (UTI) in an individual in need thereof, the method comprising administering to the individual (a) the bacteriophage of any one of embodiments 1-151, (b) the composition of any one of embodiments 1-151, or (c) the pharmaceutical composition of any one of embodiments 139-140. Numbered embodiment 153 comprises the method of embodiments 1-152, wherein the UTI is caused by E. coli. Numbered embodiment 154 comprises the method of embodiments 1-153, wherein the E. coli is a multidrug-resistant (MDR) strain. Numbered embodiment 155 comprises the method of embodiments 1-154, wherein the E. coli is an extended spectrum beta-lactamase (ESBL) strain. Numbered embodiment 156 comprises the method of embodiments 1-155, wherein the E. coli is a carbapenem-resistant strain. Numbered embodiment 157 comprises the method of embodiments 1-156, wherein the E. coli is a non-multidrug-resistant (non-MDR) strain. Numbered embodiment 158 comprises the method of embodiments 1-157, wherein the E. coli is a non-carbapenem-resistant strain. Numbered embodiment 159 comprises the method of any one of embodiments 1-158, wherein the individual is a mammal. Numbered embodiment 160 comprises the method of any one of embodiments 1-159, wherein the administering is intra-arterial, intravenous, intraurethral, intramuscular, oral, subcutaneous, inhalation, or any combination thereof. Numbered embodiment 161 comprises the method of any one of embodiments 1-160, wherein (a) the bacteriophage, (b) the composition, or (c) the pharmaceutical composition is administered at a dose of phage between 10⁶ and 10¹⁰ PFU. Numbered embodiment 162 comprises the method of any one of embodiments 1-161, wherein (a) the bacteriophage, (b) the composition, or (c) the pharmaceutical composition is administered 1, 2, 3, 4, or 5 times daily. Numbered embodiment 163 comprises the method of any one of embodiments 1-162, wherein (a) the bacteriophage, (b) the composition, or (c) the pharmaceutical composition is administered 2 times daily. Numbered embodiment 164 comprises the method of any one of embodiments 1-163, wherein (a) the bacteriophage, (b) the composition, or (c) the pharmaceutical composition is administered every 12 hours.

### EXAMPLES

### Example 1. crRNA Strain Coverage and Activity

Objectives: (1) assess the pathotype distribution of the 625 Escherichia coli (E. coli) genomes analyzed, (2) assess the strain coverages of clustered regularly spaced short palindromic repeats (CRISPR) machinery based on genomic query for the ftsA gene target and both Type I-E and Type I-F CRISPR-Cas systems, and (3) perform a functional assessment of lethality by the combined Type I-E and Type I-F ftsA-targeting CRISPR ribonucleic acid (crRNA) complexed with the leuO expression cassette.

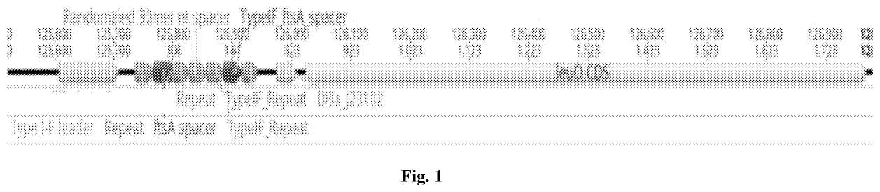

The plasmid backbone for this construct was PUC19, with the insert being comprised of the following components: native Type I-F leader, consensus Type IF repeat, Type IF ftsA spacer, Type I-F repeat, 30 nucleotide randomized sequence, Type I-E repeat, Type IE ftsA spacer, Type I-E repeat, then the biobricks promoter BBa_J23102 and then the leuO sequence from E. coli K12::MG1655, as depicted in FIG. 1. All Type I-F targeting CRISPR/Cas proteins came from E. coli strain NC101 and all Type I-E targeting machinery came from E. coli K12::MG1655.

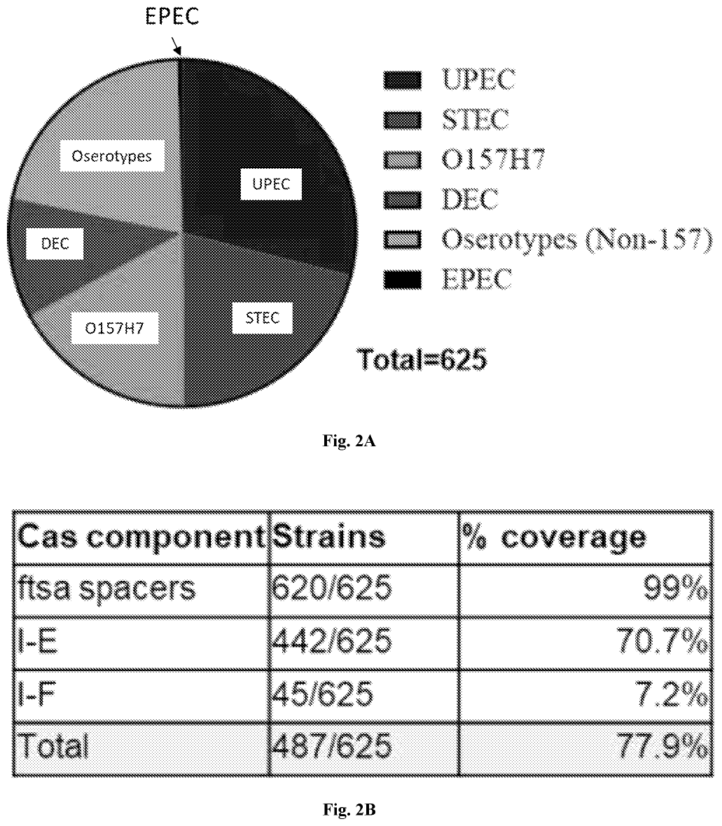

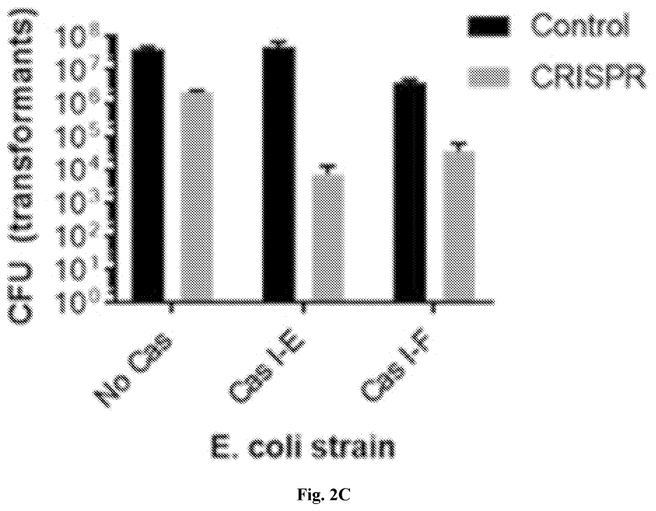

To assess the distribution of Type I CRISPR-Cas systems in E. coli, the sequences of 625 publicly available genomes across a variety of pathotypes (uropathogenic E. coli [UPEC], shiga toxin E. coli [STEC], diarrheagenic E. coli [DEC], and enteropathogenic E. coli [EPEC]) were analyzed (FIG. 2A).

Type I-E or Type I-F CRISPR-Cas systems are present in E. coli, but are mutually exclusive and additive in strain coverage. A genomic query was conducted of the 625 genomes for the ftsA gene target and both Type I-E and Type I-F CRISPR-Cas systems to determine the percent coverage (FIG. 2B). A systematic screen of E. coli genomes was performed for complete Cas operons at an 80% nucleotide identity threshold to a known Type I-E system from K12 MG1655 and a Type I-F system from strain NC101. This was performed using Geneious software version 11.1.5 using the annotation feature with an index length of 10 nucleotides. CRISPR-Cas systems in E. coli was observed at a frequency of 70.7% for Type I-E and 7.2% for Type I-F systems, totaling 77.9%.

To fulfill the third objective, a plasmid expressing each individual crRNA and a leuO expression cassette was transformed into different recipient E. coli strains containing a Type I-E or Type I-F CRISPR-Cas 3 system to determine the functional assessment of lethality after introduction of a vector expressing a Type I-E ftsA-targeting crRNA, a Type I-F ftsA-targeting crRNA co-expressed with leuO (FIG. 2C). The transformation was carried out as follows: For each strain of E. coli, a freezer stock was streaked to isolation on lysogeny broth (LB) agar and an individual colony was inoculated into 5 ml of LB medium and shaken overnight at 37° C. The cultures were back diluted into 25 ml of LB medium and grown to an A600 of 0.6 to 0.8. The cells were then pelleted and washed with ice-cold 10% glycerol 2 times before being resuspended in 150 to 350 μl of 10% glycerol. The resuspended cells (50 μL) were transformed with 50 ng of control plasmid or the Type IF/IE spacer and leuO expression plasmid using an electroporator and recovered in 300 μl of super optimal broth with catabolite repression (SOC) medium for 1 h before plating for CFU enumeration.

Type I-E and Type I-F crRNAs were designed against the conserved essential gene ftsA with 99% coverage across the 625 genomes, querying for 100% nucleotide complementarity. These data suggest a theoretical efficacy improvement in up to 77.9% of E. coli strains when CRISPR is introduced by a bacteriophage. The ftsA CRISPR for Type I-E and Type I-F targets efficiently kill E. coli with an average colony forming unit (CFU) reduction of 3.6 to >5.3-log as measured in plasmid-based transformation experiments (FIG. 2C).

### Example 2. LeuO-Dependent CRISPR Lethality in E. coli

The objective of this example was to demonstrate that expression of leuO is required for Type I-E clustered regularly spaced short palindromic repeats (CRISPR) lethality in Escherichia coli (E. coli).

M13 phagemids were derived from the base pBAD18 plasmid and modified to accommodate Type I-E CRISPR ribonucleic acids (crRNAs). The control pCRISPR control plasmid and the ftsA spacer containing variant was acquired, while variants containing the constitutive leuO expression cassette were cloned into the plasmid by restriction digestion with EcoRV and subsequent ligation. The leuO expression cassette was cloned independently into pCRISPR to create the p leuO plasmid and into p ftsA to create the p ftsA::leuO plasmid.

Phagemids were produced according to the manufacturer's protocol (New England Biolabs): Grow overnight culture of phagemid containing F′ E. coli. Inoculate a 20 mL culture in a 250 mL Erlenmeyer flask with 200 μL overnight E. coli culture. Add 1 μL phage suspension. Shake flask at 37° C., 250 rpm for 4 to 5 hours. Remove cells by centrifugation at 4500 g for 10 minutes. Transfer supernatant to a fresh tube. Repeat centrifugation. Transfer top 16 mL of supernatant to a new tube and add 4 mL of 2.5 M sodium chloride (NaCl)/20% polyethylene glycol (PEG)-8000 (w/v). Briefly mix. Precipitate phage for 1 hour or overnight at 4° C. Pellet phage by centrifugation at 12000 g for 15 minutes. Decant supernatant. Resuspend pellet in 1 mL tris-buffered saline (TBS). Transfer to an Eppendorf tube. Spin briefly to remove any cell debris. Transfer supernatant to a fresh tube. Add 200 μL of 2.5 M NaCl/20% PEG-8000. Incubate on ice for 15 to 60 minutes. Spin 12000-14000 rpm in a benchtop centrifuge for 10 minutes. Discard supernatant. Spin again briefly and remove remaining supernatant with pipette. Resuspend pellet in 1000 μL TBS.

Phagemids were produced to titers of 10⁹ transducing units per milliliter and maintained in growth media.

To verify functionality of leuO in CRISPR-mediated lethality in E. coli, a phagemid was designed encoding a leuO expression cassette to overcome the wild-type repression of the endogenous CRISPR-Cas3 operon. The designed phagemid was derived from the M13 bacteriophage, which has been shown to be non-lytic so as not to confound CRISPR-Cas3 based lethality. The phagemid also encodes a CRISPR array targeting the conserved E. coli ftsA gene, whereby expression of this array can activate and direct self-targeting of Type I-E E. coli CRISPR-Cas3 systems to elicit cell death.

Four E. coli strains (EMG2 [wild-type K12 strain], ΔHns [K12 strain lacking H-NS repression], BW25113 [engineered BW25113 strain constitutively expressing a Type I-E CRISPR-Cas operon], and BW25113ΔCRISPR [engineered BW25113 strain with deletion of entire Type I-E CRISPR-Cas operon]) were infected with a multiplicity-of-infection (MOI) of 1 for each M13 phagemid and plated on selective media to recover transduced cells and surviving colony forming units (i.e., transductants) were counted.

The following phagemids were produced to titers of 10⁹ transducing units/mL and maintained in growth media for this study: control [generic M13 transduction control], pCRISPR [phagemid that constitutively expresses non-targeting crRNA], leuO [phagemid that constitutively expresses the E. coli leuO gene]; ftsA [phagemid that constitutively expresses crRNA targeting conserved ftsA gene present in E. coli]; and ftsA::leuO [phagemid that constitutively expresses leuO gene and crRNA targeting ftsA].

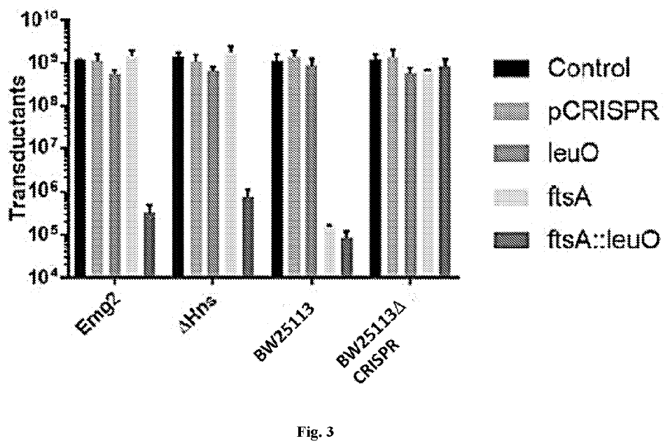

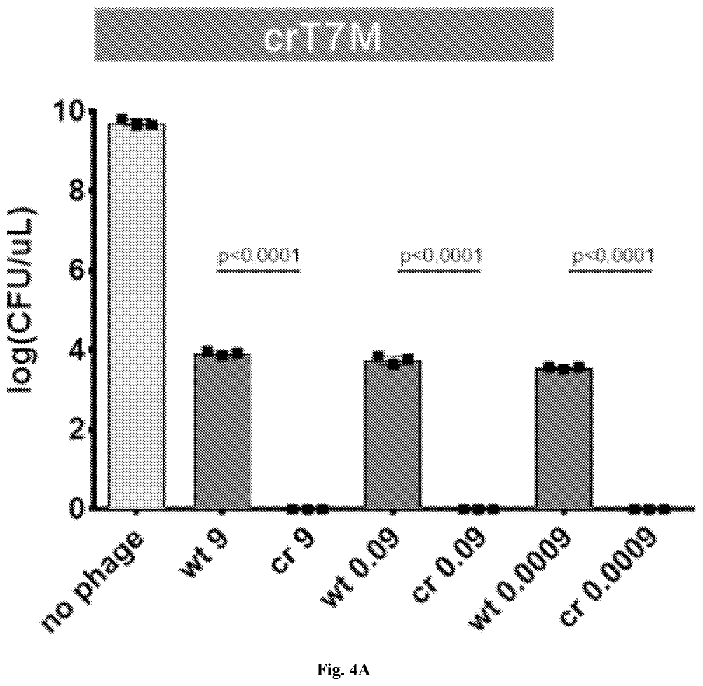

FIG. 3 and Table 1 exemplify proof-of-principle development using the validated ftsA spacer sequence and non-lytic M13 bacteriophage (“phagemid”) gene transfer. The phagemid vector encodes the ftsA repeat-spacer array, leuO expression cassette, and an M13-compatible origin of replication.

TABLE 1
M13-derived phagemid delivery of CRISPR constructs: Log CFU counts

| Experiment | Emg2 | ΔHns | BW + Cas | ΔBW + Cas |
|---|---|---|---|---|
| Phagemid | Mean (SEM) | Mean (SEM) | Mean (SEM) | Mean (SEM) |
| Experiment 1 | | | | |
| Control | 9.05 (0.007827) | 8.97 (0.01901) | 8.81 (0.016391) | 8.87 (0.01052) |
| pCRISPR | 8.00 (0.043106) | 8.82 (0.062579) | 8.89 (0.064051) | 8.84 (0.022687) |
| leuO | 8.80 (0.043106) | 8.82 (0.062579) | 8.89 (0.064051) | 8.84 (0.022687) |
| ftsA | 8.95 (0.041422) | 8.81 (0.013372) | 5.22 (0.045184) | 8.85 (0.017768) |
| ftsA::leuO | 4.77 (0.669112825) | 6.16 (0.04711) | 5.17 (0.019331) | 8.79 (0.0525) |
| Experiment 2 | | | | |
| Control | 9.04 (0.192675) | 9.34 (0.059593) | 9.32 (0) | 9.31 (0.010595) |
| pCRISPR | 9.32 (0.062469) | 9.30 (0.043575) | 9.38 (0.080684) | 9.43 (0.016092) |
| leuO | 8.87 (0.088046) | 8.99 (0.088046) | 9.18 (0.099786) | 9.00 (0.043575) |
| ftsA | 9.40 (0.034818) | 9.37 (0.073064) | 5.21 (0.066313) | 8.81 (0.033473) |
| ftsA::leuO | 5.63 (0.451544993) | 5.50 (0.543575) | 4.94 (0.098147) | 8.70 (0) |
| Experiment 3 | | | | |
| Centrol | 8.87 (0.14853) | 9.13 (0.028647) | 8.46 (0.087317) | 8.92 (0.113164) |
| pCRISPR | 8.97 (0.040373) | 8.97 (0.187473) | 8.87 (0.087747) | 8.91 (0.076024) |
| leuO | 9.06 (0.033715) | 9.18 (0.016744) | 9.15 (0) | 9.09 (0.011587) |
| ftsA | 9.25 (0.051634) | 9.19 (0.03558) | 4.23 (0.23299) | 9.14 (0.087751) |
| ftsA::leuO | 4.52 (0.189298218) | 5.37 (0.012071) | 4.26 (0.139298) | 9.18 (0) |

CFU = colonony forming units;
CRISPR = clustered regularly spaced short palindromic repeats;
SEM = standard error of the mean
Note:
Means were calculated with N=3 replicates within each experiment.

Co-delivery of leuO and the ftsA-targeting spacer resulted in reductions in the range of 3.4-log (±0.04) to 4.3-log (±0.06) compared with control across each strain except BW25113ΔCRISPR, a strain that lacks Cas3 activity, confirming that lethality is dependent on a CRISPR-Cas operon and the constructs expressed from the phagemid genome. Notably, CRISPR-Cas3 lethality by expression of a ftsA-targeting spacer alone was only observed in the constitutively expressed Type I-E CRISPR-Cas operon (BW25113) cell line, demonstrating that removal of H-NS repression alone (ΔHns) is not sufficient to rescue significant levels of endogenous CRISPR-Cas3 targeting.

The example illustrates that both the ftsA spacer and E. coli leuO are required for cell death in non-engineered, wild-type E. coli strains.

### Example 3. Bacterial Kill of Wild Type Versus CRISPR-Enhanced Phage

The objective of this example was to evaluate the ability of 3 clustered regularly spaced short palindromic repeats (CRISPR)-enhanced bacteriophages (crT7M, crT4, and crT7) to kill Escherichia coli (E. coli) compared with their respective wild-type bacteriophages

The 3 CRISPR-enhanced bacteriophages were constructed to carry a similar deoxyribonucleic acid sequence encoding functional self-targeting CRISPR ribonucleic acid (RNA) embedded in the wild-type phage genome. Generally, model phages historically used for phage display were selected and engineered at sites known to tolerate genetic manipulation (insertion/deletion). crT4 was engineered by deleting the hoc gene and replacing it with a crRNA cassette. crT7 was engineered by deleting gp0.7, gp4.3, gp4.5 and gp4.7 and replacing it with a crRNA cassette. crT7M was engineered by deleting gp0.6, 0.65, 0.7, gp4.3 and gp4.5 and replacing it with a crRNA cassette. Each phage was engineered with a crRNA cassette that contained two elements: (1) a leuO transcription factor gene derived from E. coli downstream of a synthetic promoter, and (2) a repeat-spacer-repeat encoding a crRNA targeting the ftsA gene downstream of a synthetic promoter. The orientation of the cassette elements may have differed according to the phage being used.

The crPhages and the corresponding wild-type bacteriophage were produced in E. coli (BW25113ΔCRISPR), filtered using Centricon filters (UFC 710008) and endotoxin removal columns (Thermo Pierce #88277), and adjusted to the same titer in growth media (lysogeny broth [LB] medium [Teknova #L8000]). Based on existing publicly available sequencing data, the 3 wild-type phages have significantly different genomic architecture but are all commonly known obligate lytic phages from ATCC. Initially, the timing of phage lysis was determined by monitoring optical density at 600 nm post-phage infection, from which time points for colony forming unit (CFU) enumeration were derived.

E. coli strain MG1655 (purchased from ATCC) was grown in LB medium (Teknova #L8000) and then diluted to obtain an OD600=1. Aliquots of this culture were placed in 1.7 mL tubes or 96 well plates and then combined with individual bacteriophages to create the following multiplicity of infections (MOIs), which were based on the individual titers of the produced batches:

crT7M and wild-type T7M phages: 9, 0.09, and 0.0009

crT4 and wild-type T4 phages: 6.22, 0.0622, and 0.000622

crT7 and wild-type T7 phages: 7.54, 0.0754, and 0.000754

Cultures were incubated for either 2 hours (crT7M/wild-type T7M and crT7/wild-type T7) or 5 hours (crT4/wild-type T4) at 37° C. at 250 rpm. Bacteria were then centrifuged for 1 minute at 12000×g, and washed three times with 500 μL LB medium (spinning after each wash for 1 minute at 12000×g) before being re-suspended in 500 μL LB medium.

Serial dilutions of each bacteria/phage suspension were then made and 10 μL of each dilution was streaked onto ⅓ of an LB agar plate (Teknova #L1100), which was then incubated overnight (16 to 24 hours) at 37° C. Each dilution was plated in triplicate. Surviving colonies were then enumerated.

Each crPhage was systemically compared with its corresponding wild-type bacteriophage to determine change in potency of CRISPR-enhanced phages compared with their respective wild-type bacteriophage (Table 2, Table 3, and Table 4).

TABLE 2
Log-transformed CFU/mL for crT7M versus wild type T7M phage

| | MOI = 9 | | MOI = 0.09 | | MOI = 0.0009 | |
|---|---|---|---|---|---|---|
| | Wild-type | | Wild-type | | Wild-type | |
| No Phage | T7M | crT7M | T7M | crT7M | T7M | crT7M |
| 9.653213 | 3.863323 | 0 | 3.845098 | 0 | 3.568202 | 0 |
| 9.812913 | 3.924279 | 0 | 3.770852 | 0 | 3.568202 | 0 |
| 9.662758 | 3.973128 | 0 | 3.643453 | 0 | 3.518514 | 0 |

CFU = colony-forming unit;
cr = CRISPR-enhanced phage;
MOI - multiplicity of onjection

TABLE 3
Log-transformed CFU/mL for crT4 versus wild-type T4 phage

| | | MOI = 6.22 | | MOI = 0.0622 | | MOI = 0.000622 | |
|---|---|---|---|---|---|---|---|
| | | Wild-type | | Wild-type | | Wild-type | |
| | No Phage | T4 | crT4 | T4 | crT4 | T4 | crT4 |
| Experiment # 1 | 9.80618 | 6.477121 | 0 | 5.69897 | 0 | 0 | 4 |
| | 9.724276 | 5.826075 | 0 | 5.518514 | 0 | 0 | 4.845098 |
| | 9.770852 | 5.716003 | 0 | 5.230449 | 0 | 4 | 4 |
| Experiment #2 | 10.14613 | 5.176091 | 0 | 3.778151 | 0 | 3.90309 | 0 |
| | 10.32222 | 5.113944 | 0 | 4.255272 | 0 | 3.544068 | 0 |
| | 12.62325 | 3.69897 | 0 | 4.113944 | 0 | 3.623249 | 0 |
| Experiment #3 | 10.23045 | 5.838849 | 2.30103 | 4.485722 | 0 | 3.447158 | 3 |
| | 19.32222 | 5.763428 | 0 | 4.30103 | 6 | 3.50515 | 2.60206 |
| | 10.27875 | 5.863323 | 0 | 4.30103 | 0 | 3.672098 | 3.255272 |

CFU = colony-forming unit;
cr = CRISPR-enhaneed phage;
MOI = multiplicity of infection

TABLE 4
Log-transformed CFU/mL for crT7 versus wild-type T7 phage

| | | MOI = 7.54 | | MOI = 0.0754 | | MOI = 0.000754 | |
|---|---|---|---|---|---|---|---|
| | | Wild-type | | Wild-type | | Wild-type | |
| | No Phage | T7 | crT7 | T7 | crT7 | T7 | crT7 |
| Experiment #1 | 9.462398 | 4.477121 | 3.556303 | 3.863323 | 2.778151 | 4.322219 | 3.361728 |
| | 9.78533 | 4.491362 | 3.643453 | 3.812913 | 3.544068 | 4.176001 | 3.041393 |
| | 9.591065 | 4.431364 | 3.662758 | 3.681241 | 3.30103 | 4.230449 | 3.447158 |
| Experiment #2 | 9.331479 | 4.462398 | 3.643453 | 4.380211 | 3.414973 | 3.845008 | 3.30103 |
| | 9.414973 | 4.633469 | 3.591065 | 3.90309 | 3.39794 | 4.041393 | 3.113943 |
| | 9.544068 | 4.361728 | 3.662758 | —^{a} | 3.462398 | 4.041393 | 3.361728 |

CFU = colony-forming unit;
cr = CRISPR-enhanced phage;
MOI = multiplicity of infection
^{a}Data not available due to experimental error.

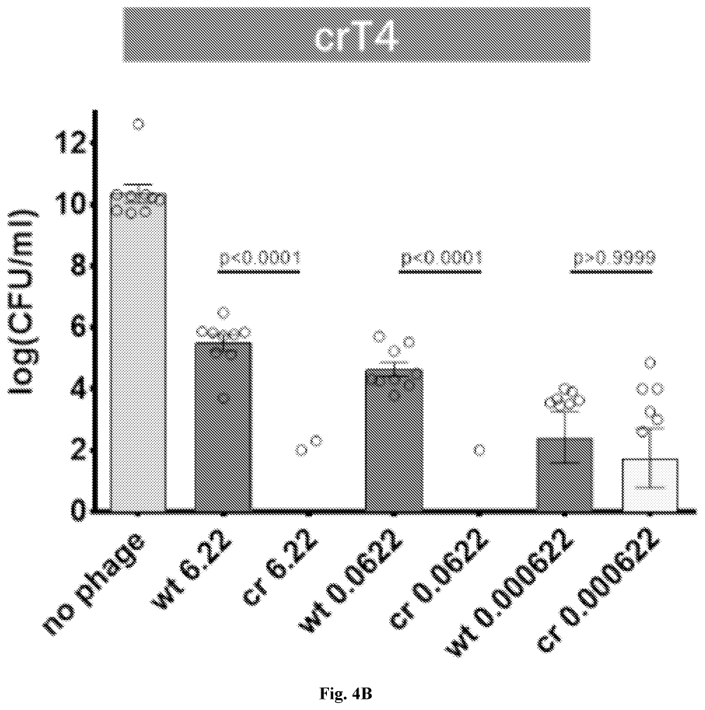

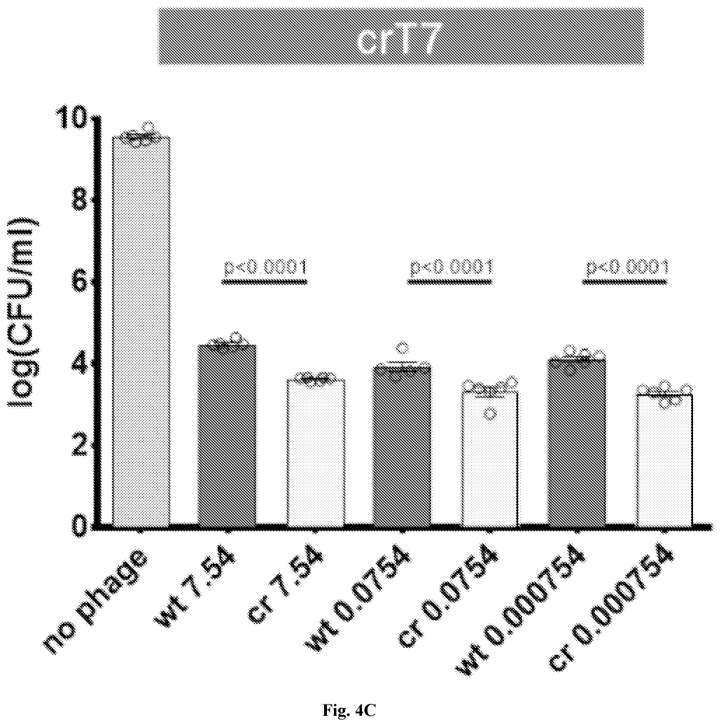

Significant differences were observed in CFU reduction across all 3 crPhage to wild-type phage comparisons (FIG. 4A-FIG. 4C), including up to an approximately 4-log improvement of crT7M (FIG. 4A), approximately 4.5-log improvement of crT4 (FIG. 4B), and approximately 1-log improvement of crT7 (FIG. 4C) activities.

These data exemplify that CRISPR-enhanced bacteriophages better eliminate the target E. coli population, in contrast to the wild-type bacteriophage.

### Example 4. Activity of LBP-EC01 Clinical E. coli crPhage Cocktail

The objective of this example was to evaluate the host range and durability of each individual clustered regularly interspaced short palindromic repeats (CRISPR)-enhanced phage (crPhage) and the 3-phage combined crPhage cocktail

LBP-EC01 was generated containing phages p004k-5, p0031-8, and p00ex-2. Three hundred fifty-two Escherichia coli (E. coli) strains used in the study were purchased from International Health Management Associates (IHMA) and were isolates from patients located across the United States who were diagnosed with urinary tract infections (UTIs). These isolates were used to assess the host range of the phage cocktail and dose response. One hundred and seventy-six isolates from various sources were also used to assess individual phage host range (Table 5).

TABLE 5
Strain panel trait overview
Isolates from Various Sources

| | |
|---|---|
| Pathogenic | 160 |
| UTI | 112 |
| CDC | 22 |
| AIEC | 20 |
| UPEC/NIEC | 6 |
| Non-Pathogenic | 16 |
| Total | 176 |
| IMHA Strains | |
| UTI | 352 |
| MDR | 71 |
| Total | 352 |

AIEC = adherent invasive E. coli;
CDC = provided through the Centers for Disease Control and Prevention;
IMHA = International Health Management Associates;
MDR = multidrug resistance;
NIEC = non-adherent invasive E. coli;
UPEC = uropathogenic E. coli;
UTI = uinary tract infection

Each crPhage was produced by standard lytic amplification (amplification occurred for 4 hours post-infection), filtration (PDP8 and PDE2 clarification filters and EKV sterilization filter), and then each crPhage was purified and suspended in Lactated Ringer's solution. All experiments were conducted in lysogeny broth (LB) medium supplemented with 10 mM MgCl₂ and 10 mM CaCl₂. Bacterial strains used in these experiments were purchased from IRMA and included a contemporaneous collection of E. coli strains isolated in 2018 from patients spread geographically across the United States that were diagnosed with UTIs.

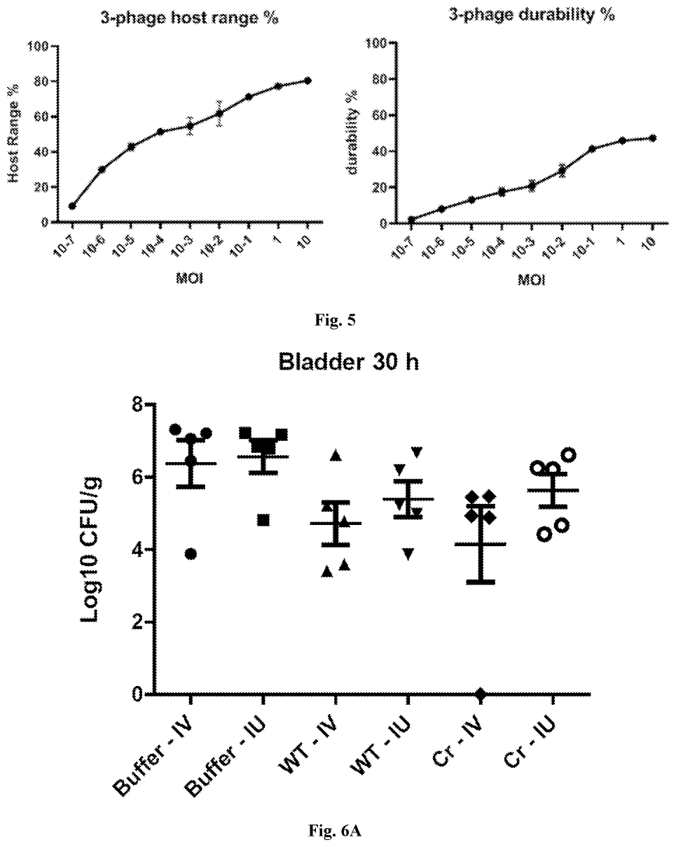

Each E. coli strain was placed in a microtiter plate either alone or with the crPhage cocktail at the MOIs indicated in FIG. 5 (10⁻⁷ to 10). The cultures were incubated at 37° C. for 18 hours in a plate reader to monitor growth of populations by optical density (OD; 600 nm).

To calculate “hits,” ODs were plotted over time to generate an area under the curve (AUC) for each strain with and without crPhage treatment. The ratio of these 2 AUCs (with and without crPhage treatment) provided an AUC score. If the AUC ratio was ≤0.65 it was considered a “hit.” The host range hits were calculated as the percentage of total strains in the panel in which the AUC ratio was ≤0.65. Durability of effect was based on strains which did not achieve an OD600 ≥0.4 at 18 hours. The durability percentage was calculated as the number of durability hits relative to the total number of strains in the panel.

Each of the individual crPhages exhibited a host range between ˜20% and 57% and durability % from ˜3% to 15% against the Locus internal strain panel. The combined LBP-EC01 cocktail host range was ˜82% using a multiplicity of infection of 10 against the contemporaneous UTI strain panel from IHMA (strains A, B, C, D) (Table 6). The dose response curves were generated against IHMA panels A and B, demonstrating a relatively linear dose response between MOI 10⁻⁴ and MOI 10, but dropped rapidly below a host range of 50% at MOIs below 10⁻⁴. Similarly, the durability percentage response dropped rapidly below 40% of total strains at MOI <10⁻¹ but was stable between MOI 10⁻¹ and MOI 10 (FIG. 5).

TABLE 6
Host Range and Durability % for LBP-EC01 at MOI 10
Host Range and Durability % for individual phages at MOI 10

| | PTA-126324 | PTA-126319 | PTA-126315 |
|---|---|---|---|
| Host Range % | 57.40 | 56.21 | 20.59 |
| Durability % | 15.38 | 10.06 | 3.53 |

Host Range and Durability % for LBP-EC01
at MOI 10 against IHMA A, B, C, D strains

| | |
|---|---|
| | Cocktail |
| Host Range % | 82.1 |
| Durability % | 50.2 |

IHMA = International Health Management Associates;
MOI = multiplicity of infection

The example exemplifies: (1) The host range of the LBP-EC01 cocktail against the full IHMA strain panel is 82.1% and the durability % is 50.2% (2) Against the A and B subset of the IHMA strain panel, the dose response curves demonstrate a linear dose response between MOI 10 and MOI 10⁻⁴ (3) Against the A and B subset of the IHMA strain panel, MOIs below 10⁻⁴ resulted in host range dropping more rapidly below 50% (4) Against the A and B subset of the IHMA strain panel, the durability percentage response was stable between MOI 10⁻¹ and MOI 10 but dropped significantly below 40% of total strains at MOI <10⁻¹.

### Example 5. Evaluation of Phages Against E. coli in the Mouse UTI Model

The objective of this example was to evaluate two phages, WT and crPhage cocktail, against E. coli LFP 527, also referred to interchangeably as “Ec 527” and “NC101”, in the mouse UTI infection model.

Test phages: Buffer only, WT cocktail, and crPhage cocktail. The materials were stored refrigerated (2-8° C.) and were kept on ice during dosing.

Preparation of inoculum: E. coli strain LFP 527 colonies from an overnight LB agar plate were transferred to 10 mL of LB broth in 250-mL Erlenmeyer flasks. Flasks were incubated statically at 37° C. for 18 h. After the first overnight growth, a 100-4, aliquot of the suspension from each flask was transferred to 25 mL of LB broth in 250-mL Erlenmeyer flasks. Flasks were incubated statically at 37° C. for 24 h. Each suspension was transferred to sterile conical tubes and centrifuged at 6,200 g for 5 min. The supernatants were decanted and the pellets resuspended in ˜2 mL of sterile PBS. The target concentration of the suspension was 1E+10 CFU/mL. The actual titer of the suspension was determined using the dilution plate count method and was 9.6E+09 CFU/mL.

Animals: The female mice (Mus musculus), strain C3H/Hen, were obtained from Charles River Laboratories, Stone Ridge, N.Y. Mice were 57-days-old on the day of infection.

TABLE 7
Dosing regimen

| | | Dose | Dose | No. |
|---|---|---|---|---|
| Grp | Treatment | route | (h after infection) | mice |
| 1 | Infected control-24 h | NA | NA | 5 |
| 2 | Infected control-Buffer | IV | 24, 36, 48, 60 and 72 | 10 |
| 3 | | IU | 24, 36, 48, 60 and 72 | 10 |
| 4 | WT Cocktail | IV | 24, 36, 48, 60 and 72 | 10 |
| 5 | | IU | 24, 36, 48, 60 and 72 | 10 |
| 6 | Cr Cocktail | IV | 24, 36, 48, 60 and 72 | 10 |
| 7 | | IU | 24, 36, 48, 60 and 72 | 10 |

Infection procedure: Each mouse was placed in an induction chamber filled with isoflurane carried in O₂ to initiate anesthesia. The mouse was then placed on the anesthesia board, ventral side up, with the nose inserted in a nosecone supplied with isoflurane. The lower abdomen was gently massaged to expel urine from the bladder. Using a 30 G×½ in. needle covered with polyethylene tubing (0.61 mm O.D.) and affixed to a 1 mL syringe, 50 μL of the inoculum was slowly injected into the bladder. After infection the mouse was returned to its cage.

Dosing procedure: Mice in Groups 2, 4 and 6 were dosed with 100 μL of each treatment by IV injection in a tail vein. Group 3, 5 and 7 mice were dosed with 50 μL of each treatment by IU instillation. The instillation procedure for dosing was the same as that used for infection. Doses were administered at 24, 36, 48, 60 and 72 h after infection.

Tissue collection and processing: At 30 and 78 h after infection, five mice per group were euthanized, the bladders and kidneys were removed and placed in sterile 13-mL flip-top tubes. The tubes were weighed and 1 mL of sterile PBS was added. The bladders were homogenized using the Tekmar Tissumizer and the kidneys using the Polytron 3100. Serial dilutions of the homogenates were plated on TSA and incubated at 37° C. overnight. Colony counts were used to calculate the CFU/g tissue.

Statistical analysis: One-way ANOVA with Dunnett's post test comparing the buffer control to the phage treatments was performed on the log 10 CFU/g tissue data using GraphPad Prism version 5.04 for Windows, Graphpad Software, San Diego, Calif.

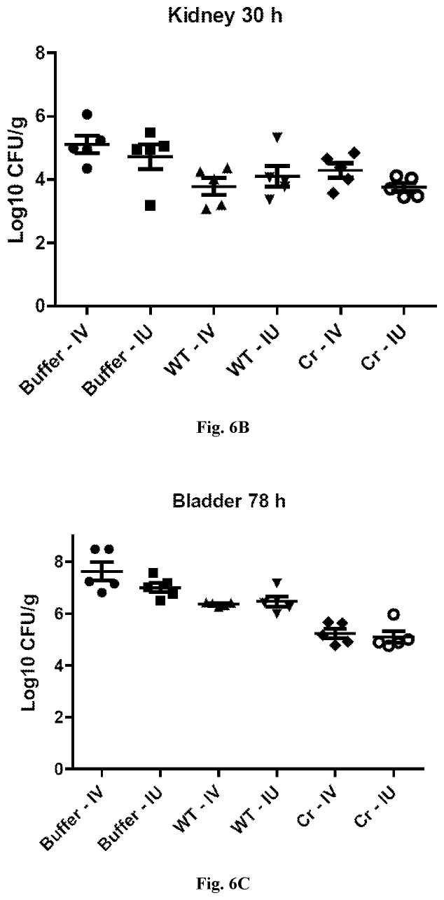

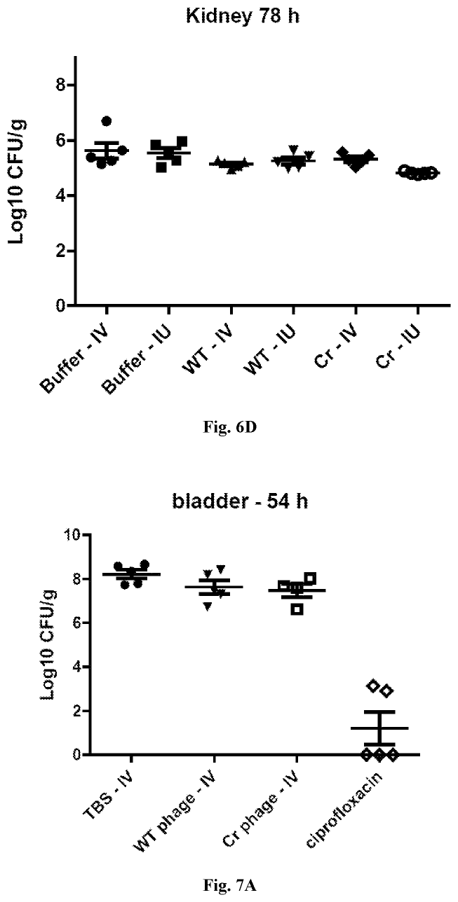

Bacterial density in the bladder and kidneys: The density of bacteria in the bladder and kidneys at the start of treatment (24 h after infection) was 5.97±1.80 and 4.91±1.54 log₁₀ CFU/g tissue, respectively. At 6 h after the first treatment (30 h after infection), neither the IV or IU-dosed phage treatments reduced the bacterial density in the bladder compared to the buffer control treatment (FIG. 6A, Table 8). In the kidneys, only the IV-dosed WT phage resulted in a significant reduction in bacterial density compared to the buffer control treatment (FIG. 6B).

At 6 h after the fifth treatment (78 h after infection), the IV-dosed WT and crPhage treatments significantly reduced bacterial density in the bladder compared to the buffer control treatment (FIG. 6C). Among the IV-dosed mice, both the WT and crPhage treatments significantly reduced the density of bacteria in the bladder compared to the buffer control treatment. Among the IU-dosed groups, only the crPhage treatment significantly reduced bacterial density in the bladder and the kidneys (FIG. 6D).

TABLE 8
Density of E. coli (LFP 527) recovered from bladder and kidneys
of infected mice treated with buffer, WT cocktail or Cr (crPhage)
cocktail administered by IV injection or IU instillation

| | | 30 h | | 78 h | |
|---|---|---|---|---|---|
| Grp | Treatment | bladder | kidney | bladder | kidney |

IV dose

| | | | | | |
|---|---|---|---|---|---|
| 2 | Buffer control | 6.38 ± 1.44 | 5.11 ± 0.96 | 7.63 ± 0.79 | 5.62 ± 0.62 |
| 4 | WT cocktail | 4.72 ± 1.30 | 3.78 ± 0.54**^{a} | 6.36 ± 0.07** | 5.14 ± 0.13 |
| 6 | Cr cocktail | 4.14 ± 2.33 | 4.29 ± 0.46 | 5.23 ± 0.40*** | 5.32 ± 0.21 |

IU dose

| | | | | | |
|---|---|---|---|---|---|
| 3 | Buffer control | 6.57 ± 1.00 | 4.72 ± 1.04 | 7.00 ± 0.41 | 5.53 ± 0.39 |
| 5 | WT cocktail | 5.39 ± 1.09 | 4.11 ± 0.69 | 6.47 ± 0.43 | 5.25 ± 0.28 |
| 7 | Cr cocktail | 5.64 ± 0.91 | 3.76 ± 0.38 | 5.10 ± 0.49** | 4.82 ± 0.04** |

^{a}*significant at P < 0.05;
**significant at P < 0.01;
***significant at P < 0.001

### Example 6. Evaluation of Phages Against E. coli in the Mouse UTI Model

The objective of this example was to evaluate two phage cocktails, wtPhage Cocktail and crPhage Cocktail (derived from the same wtPhage), against E. coli strain LFP 527 in the mouse UTI infection model.

Test phages: Buffer only, WT cocktail, and crPhage cocktail. The materials were stored refrigerated (2-8° C.) and were kept on ice during dosing.

Standard: Ciprofloxacin hydrochloride (MP Biomedicals, LLC, Cat. No. 199020, Lot Q9860) was formulated in sterile saline at a concentration of 1 mg/mL.

Preparation of inoculum: E. coli strain LFP 527 colonies from an overnight LB agar plate were transferred to 10 mL of LB broth in 250-mL Erlenmeyer flasks. Flasks were incubated statically at 37° C. for 18 h. After the first overnight growth, a 100-μL aliquot of the suspension from each flask was transferred to 25 mL of LB broth in 250-mL Erlenmeyer flasks. Flasks were incubated statically at 37° C. for 24 h. Each suspension was transferred to sterile conical tubes and centrifuged at 6,200 g for 5 min. The supernatants were decanted and the pellets resuspended in ˜2 mL of sterile PBS. The target concentration of the suspension was 1E+10 CFU/mL. The actual titer of the suspension was determined using the dilution plate count method and was 1.1E+10 CFU/mL.

Animals: The female mice (Mus musculus), strain C3H/Hen, were obtained from Charles River Laboratories, Stone Ridge, N.Y. Mice were 53-days-old on the day of infection.

TABLE 9
Dosing regimen

| | | | Dose | |
|---|---|---|---|---|
| Grp | Treatment | Dose route | (h after infection) | No. mice |
| 1 | Infected control-48 h | NA | NA | 5 |
| 2 | Infected control-TBS | IV | 48, 60, 72, 84 and 96 | 10 |
| 3 | | IU | 48, 60, 72, 84 and 96 | 10 |
| 4 | | IV + IU | 48, 60, 72, 84 and 96 | 10 |
| 5 | Phage #1 | IV | 48, 60, 72, 84 and 96 | 10 |
| 6 | | IU | 48, 60, 72, 84 and 96 | 10 |
| 7 | | IV + IU | 48, 60, 72, 84 and 96 | 10 |
| 8 | Phage #2 | IV | 48, 60, 72, 84 and 96 | 10 |
| 9 | | IU | 48, 60, 72, 84 and 96 | 10 |
| 10 | | IV + IU | 48, 60, 72, 84 and 96 | 10 |
| 11 | Ciprofloxacin-5 mg/kg | IV | 48, 60, 72, 84 and 96 | 10 |

Infection procedure: Each mouse was placed in an induction chamber filled with isoflurane carried in O₂ to initiate anesthesia. The mouse was then placed on an anesthesia board, ventral side up, with the nose inserted in a nosecone supplied with isoflurane. The lower abdomen was gently massaged to expel urine from the bladder. Using a 30 G×½ in. needle covered with polyethylene tubing (0.61 mm O.D.) and affixed to a 1 mL syringe, 50 μL of the inoculum was slowly injected into the bladder. After infection the mouse was returned to its cage.

Dosing procedure: Mice in Groups 2, 5, 8 and 11 were dosed with 100 μL of the appropriate treatment by IV injection in a tail vein. Group 3, 6 and 9 mice were dosed with 50 μL of each treatment by IU instillation. The instillation procedure for dosing was the same as that used for infection. Mice in Groups 4, 7 and 10 were dosed by IU instillation followed by IV injection. Doses were administered at 48, 60, 72, 84 and 96 h after infection.

Tissue collection and processing: At 48 (Group 1 only), 54 and 102 h after infection, five mice per group were euthanized, the bladders, kidneys and spleens were removed and placed in sterile 13-mL flip-top tubes. The tubes were weighed and 1 mL of sterile PBS was added. The bladders and spleens were homogenized using the Tekmar Tissumizer and the kidneys using the Polytron 3100. Serial dilutions of the homogenates were plated on TSA and incubated at 37° C. overnight. Colony counts were used to calculate the CFU/g tissue.

Statistical analysis: One-way ANOVA with Dunnett's post-test comparing the buffer control to the phage treatments was performed on the log 10 CFU/g tissue data using GraphPad Prism version 5.04 for Windows, Graphpad Software, San Diego, Calif.

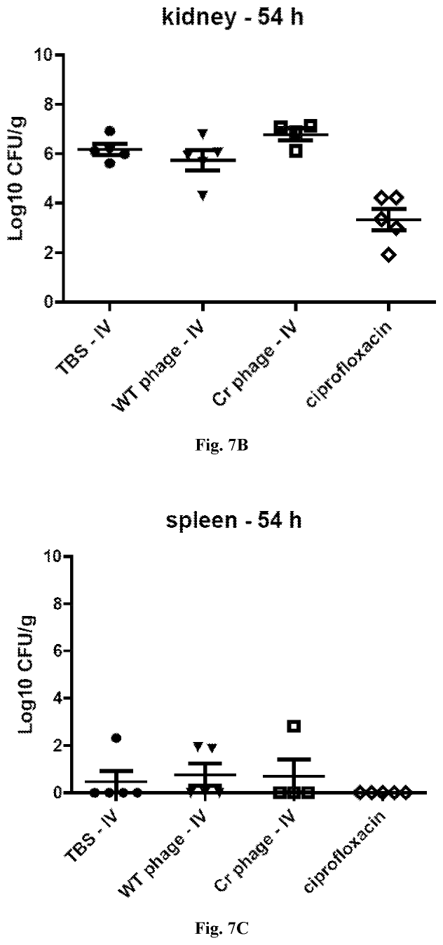

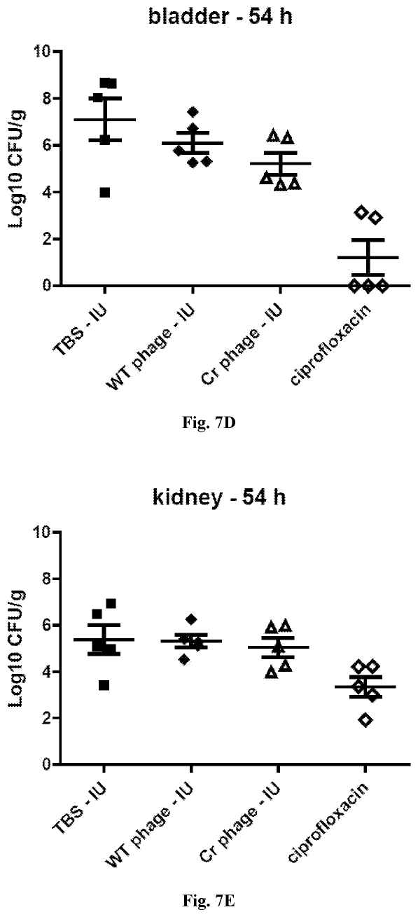

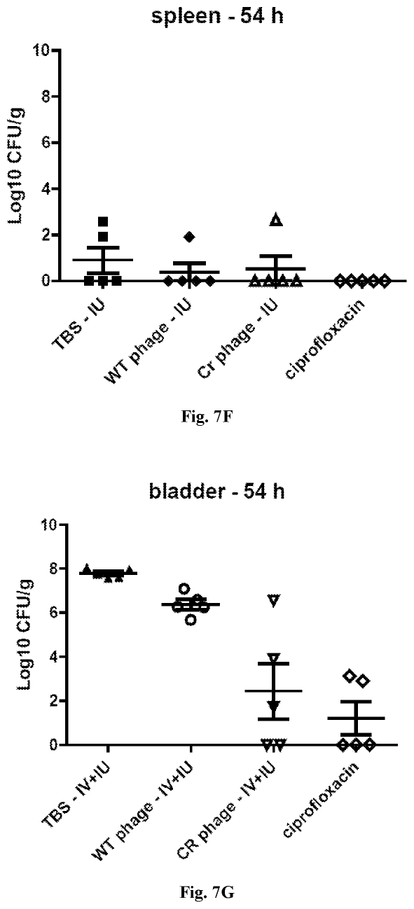

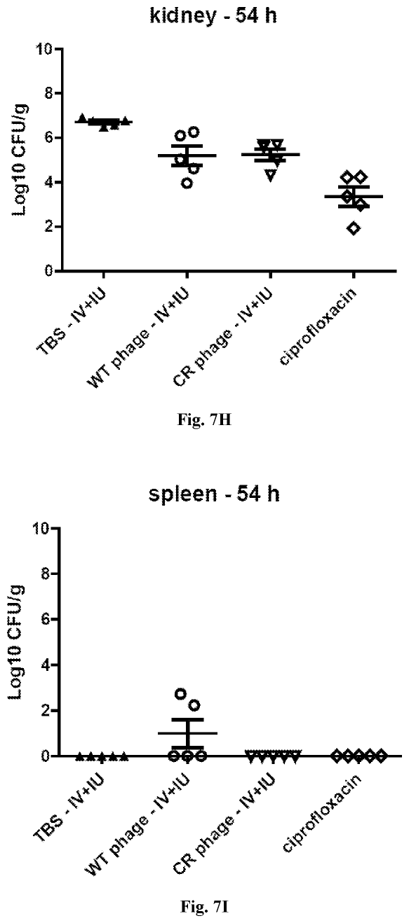

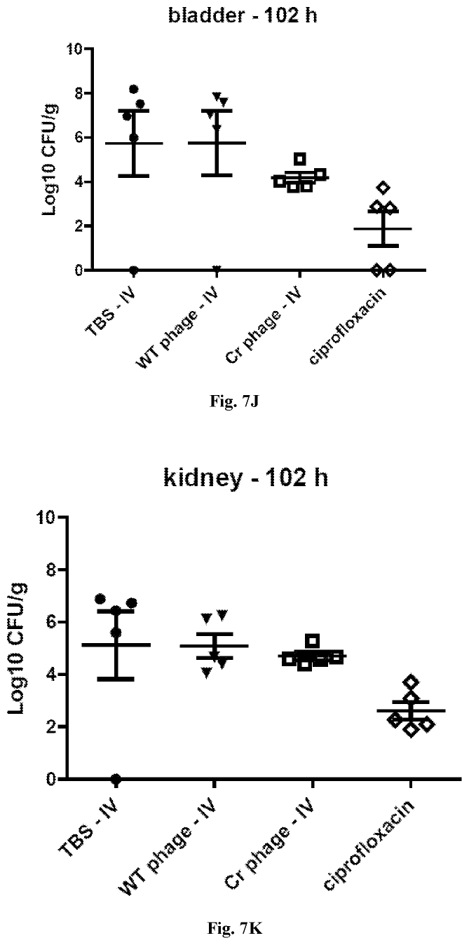

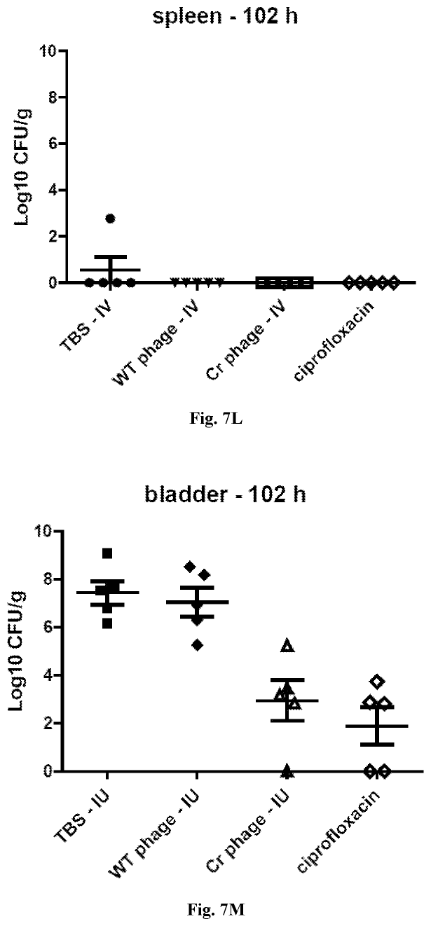

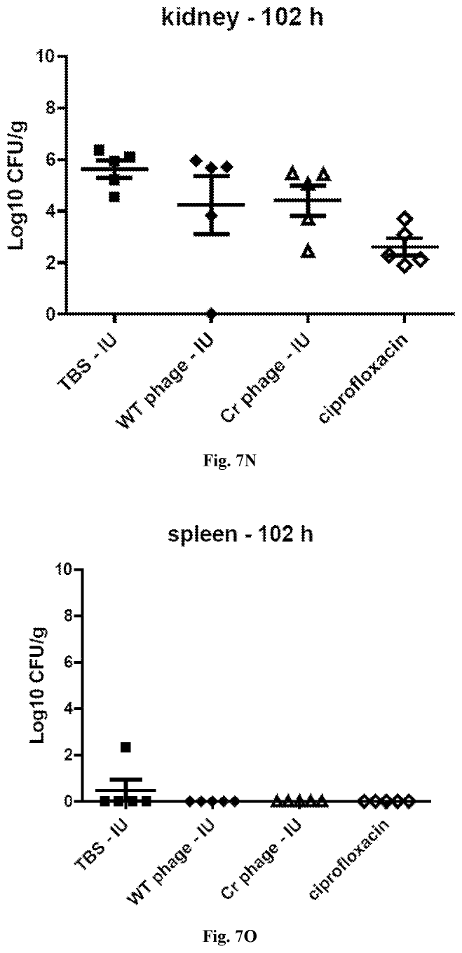

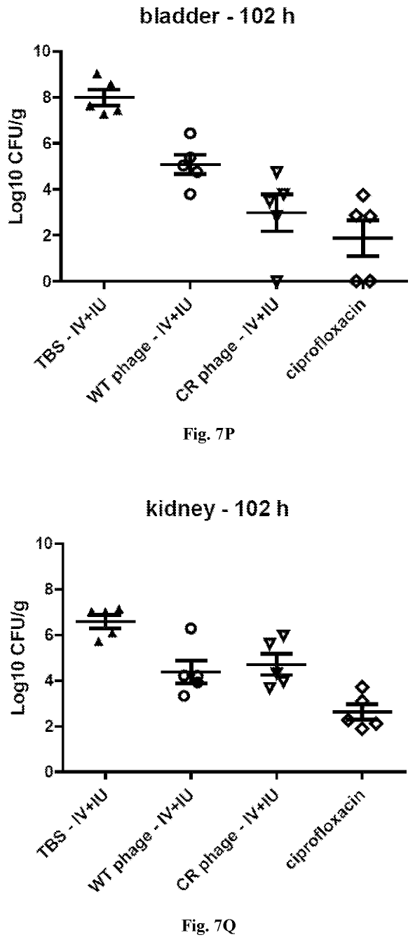

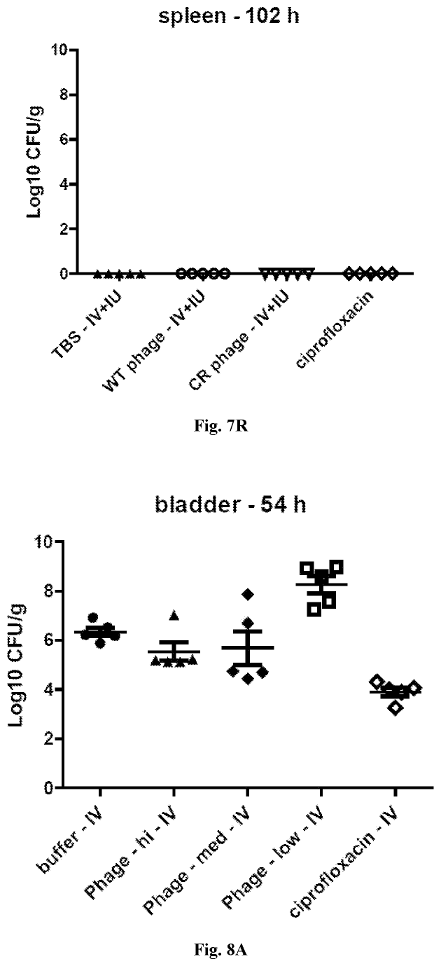

Bacterial density in the bladder and kidneys: The results are exemplified in FIG. 7A-FIG. 7R, and Table 10. The density of bacteria in the bladder and kidneys at the start of treatment (48 h after infection) was 7.95±1.09 and 6.94±1.74 log₁₀ CFU/g tissue, respectively. No bacteria was recovered from the spleens at this time point.

At 6 h after the first treatment (54 h after infection), IV+IU dosing with the crPhage resulted in a significant reduction of bacteria in the bladder and kidneys compared to the buffer control treatment. Dosing by this route with the WT phage resulted in a significant reduction in the kidneys but not the bladder. No significant reduction was observed in the groups treated with either of the phages by the IV or IU only routes.

At 6 h after the fifth treatment (102 h after infection), both the WT and crPhages significantly reduced bacterial density in the bladder and kidneys when administered by the IV+IU route. The crPhage also significantly reduced bacterial density in the bladder when administered IU.

The standard, ciprofloxacin, significantly reduced bacteria in the bladder and kidneys when compared to all of the buffer control treatments with the exception of the comparison to the buffer treatment dosed by the IV route at 102 h in both the bladder and kidneys.

Data for the spleen tissue indicated that very few bacteria disseminated from the bladder and kidneys. Bacterial colonies were only recovered from ten mice at 54 h after infection and from two mice at 102 h after infection.

TABLE 10
Density of E. coli (LFP 527) recovered from bladder, kidneys and spleens of
infected mice treated with TBS buffer, WT cocktail or crPhage cocktail administered by
IV injection, IU instillation or both. Ciprofloxacin was administered by IV injection only.

| | | 54 h | | | 102 h | | |
|---|---|---|---|---|---|---|---|
| Grp | Treatment | bladder | kidney | spleen | bladder | kidney | spleen |
| 1 | Infected control—48 h | 7.96 ± 1.09 | 6.94 ± 1.74 | 0 | | | |
| IV | | | | | | | |
| 3 | Infected control—TBS | 8.22 ± 0.43 | 6.18 ± 0.48 | 0.46 ± 1.03 | 5.73 ± 3.31 | 5.13 ± 2.91 | 0.55 ± 1.24 |
| 5 | WT phage cock tall | 7.83 ± 1.15 | 5.74 ± 0.92 | 0.76 ± 1.04 | 5.76 ± 3.27 | 5.10 ± 1.02 | 0 |
| 8 | Cr phage cocktail | 7.48 ± 0.74 | 6.79 ± 0.47 | 0.70 ± 1.41 | 4.19 ± 0.52 | 4.70 ± 0.33 | 0 |
| 11 | ciprofloxacin | 1.21 ± 1.50 *** | 3.35 ± 0.96 *** | 0 | 1.89 ± 1.76 | 0 | G |
| IU | | | | | | | |
| 3 | Infected control—TBS | 7.12 ± 1.80 | 5.38 ± 1.40 | 0.90 ± 1.25 | 7.44 * 1.09 | 5.63 * 0.74 | 0.47 ± 1.05 |
| 6 | WT phage cocktail | 3.09 ± 0.85 | 5.33 ± 0.62 | 0.38 ± 0.86 | 7.04 ± 1.35 | 4.24 ± 2.52 | 0 |
| 9 | Cr phage cocktail | 5.21 ± 1.10 | 5.04 ± 0.92 | 0.53 ± 1.19 | 2.94 * 1.89 *** | 4.42 ± 1.32 | 0 |
| 11 | ciprofloxacin | 1.21 ± 1.50 ** | 3.35 ± 0.96 * | 0 | 1.89 ± 1.76 *** | 2.62 ± 0.76 | 0 |
| IV + IU | | | | | | | |
| 5 | Infected control—TBS | 7.80 ± 0.23 | 6.71 ± 0.17 | 0 | 7.99 ± 0.77 | 6.59 ± 0.64 | 0 |
| 7 | WT phage cocktail | 6.38 ± 0.63 | 5.19 ± 0.98 * | 0.99 ± 1.37 | 5.08 ± 0.96 * | 4.39 ± 1.12 ** | 0 |
| 10 | Cr phage cocktail | 2.44 ± 2.53 *** | 5.24 ± 0.59 * | 0 | 2.98 ± 1.80 *** | 4.71 ± 0.95 * | 0 |
| 11 | ciprofloxacin | 1.21 + 1.50 *** | 3.35 ± 0.96 *** | 0 | 1.89 + 1.76 *** | 2.62 ± 0.76 ** | 0 |

* = significant at P < 0.05;
** = significant at P < 0.01;
*** = significant at P < 0.001

### Example 7. Evaluation of Phages Against E. coli in the Mouse UTI Model

The objective of this example was to evaluate two phages products, phage cocktail and Pyophage (a commercially available cocktail of bacteriophage, sold by Eliava Biopreparations), against E. coli strain LFP 527 in the mouse UTI infection model.

Test phages: Buffer only, phage cocktail—High, Med, Low, and Pyophage. The materials were stored refrigerated (2-8° C.) and were kept on ice during dosing. The Phage cocktail was a mixture of phages p0033L-10 (ATCC No. PTA-126316), p004k-5 (ATCC No. PTA-126319), and p0071-16 (ATCC No. PTA-126320) at concentrations of 2×10¹¹ plaque forming units (PFU)/mL/phage (“high”), 2×10⁹ PFU/mL/phage (“medium”), or 2×10⁷ PFU/mL/phage (“low”). A 50 μl dose thus provided 1×10¹⁰ PFU/phage (“high”), 1×10⁸ PFU/phage (“medium”), or 1×10⁶ PFU/phage (“low”).

Standard: Ciprofloxacin hydrochloride (MP Biomedicals, LLC, Cat. No. 199020, Lot Q9860) was formulated in sterile saline at a concentration of 2.5 mg/mL.

Preparation of inoculum: E. coli strain LFP 527 colonies from an overnight LB agar plate were transferred to 10 mL of LB broth in 250-mL Erlenmeyer flasks. Flasks were incubated statically at 37° C. for 18 h. After the first overnight growth, a 100-μL aliquot of the suspension from each flask was transferred to 25 mL of LB broth in 250-mL Erlenmeyer flasks. Flasks were incubated statically at 37° C. for 24 h. Each suspension was transferred to sterile conical tubes and centrifuged at 6,200 g for 5 min. The supernatants were decanted and the pellets resuspended in ˜2 mL of sterile PBS. The target concentration of the suspension was 1E+10 CFU/mL. The actual titer of the suspension was determined using the dilution plate count method and was 8.8E+9 CFU/mL.

Animals: The female mice (Mus musculus), strain C3H/Hen, were obtained from Charles River Laboratories, Stone Ridge, N.Y. Mice were 56-days-old on the day of infection.

TABLE 11
Dosing regimen of crPhage (Locus phage) cocktail and pyophage

| | | Dose | Dose | |
|---|---|---|---|---|
| Grp | Treatment | route | (h after infection) | No. mice |
| 1 | Infected control-48 h | NA | NA | 5 |
| 2 | Infected control-buffer | IV | 48, 60, 72, 84 and 96 | 10 |
| 3 | | IU | 48, 60, 72, 84 and 96 | 10 |
| 4 | Locus phage-high | IV | 48, 60, 72, 84 and 96 | 10 |
| 5 | | IU | 48, 60, 72, 84 and 96 | 10 |
| 6 | Locus phage-med | IV | 48, 60, 72, 84 and 96 | 10 |
| 7 | | IU | 48, 60, 72, 84 and 96 | 10 |
| 8 | Locus phage-low | IV | 48, 60, 72, 84 and 96 | 10 |
| 9 | | IU | 48, 60, 72, 84 and 96 | 10 |
| 10 | Pyophage | IU | 48, 60, 72, 84 and 96 | 10 |
| 11 | ciprofloxacin | IV | 48, 60, 72, 84 and 96 | 10 |

Infection procedure: Each mouse was placed in an induction chamber filled with isoflurane carried in O₂ to initiate anesthesia. The mouse was then placed on an anesthesia board, ventral side up, with the nose inserted in a nosecone supplied with isoflurane. The lower abdomen was gently massaged to expel urine from the bladder. Using a 30 G×½ in. needle covered with polyethylene tubing (0.61 mm O.D.) and affixed to a 1 mL syringe, 50 μL of the inoculum was slowly injected into the bladder. After infection the mouse was returned to its cage.

Dosing procedure: Mice in Groups 2, 4, 6, 8 and 11 were dosed with 50 μL of the appropriate treatment by IV injection in a tail vein. Group 3, 5, 7, 9 and 10 mice were dosed with 50 μL of each treatment by IU instillation. The instillation procedure for dosing was the same as that used for infection. Doses were administered at 48, 60, 72, 84 and 96 h after infection.

Tissue collection and processing: At 48 (Group 1 only), 54 and 102 h after infection, five mice per group were euthanized, the bladders and kidneys were removed and placed in sterile 13-mL flip-top tubes. The tubes were weighed and a volume of sterile PBS equivalent to 30× and 4× the weight of the bladders and kidneys, respectively, was added. The bladders were homogenized using the Tekmar Tissumizer and the kidneys using the Polytron 3100. Serial dilutions of the homogenates were plated on TSA and incubated at 37° C. overnight. Colony counts were used to calculate the CFU/g tissue.

Statistical analysis: One-way ANOVA with Dunnett's post-test comparing the buffer control to the phage treatments was performed on the log 10 CFU/g tissue data using GraphPad Prism version 5.04 for Windows, Graphpad Software, San Diego, Calif.

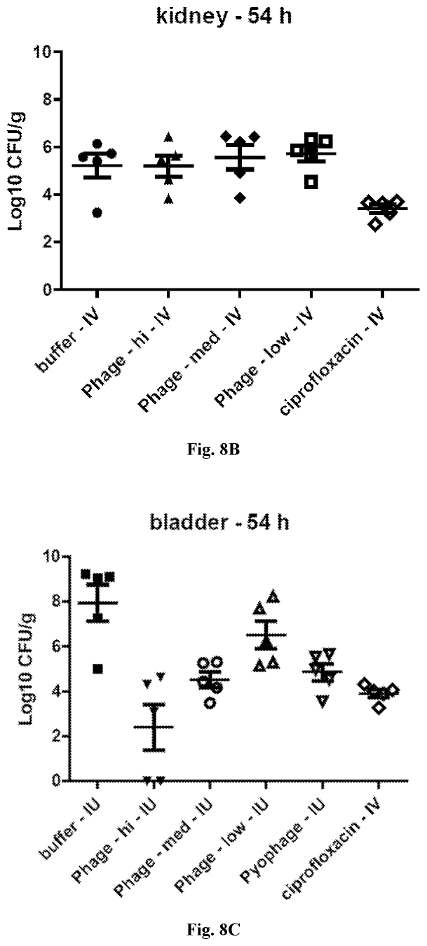

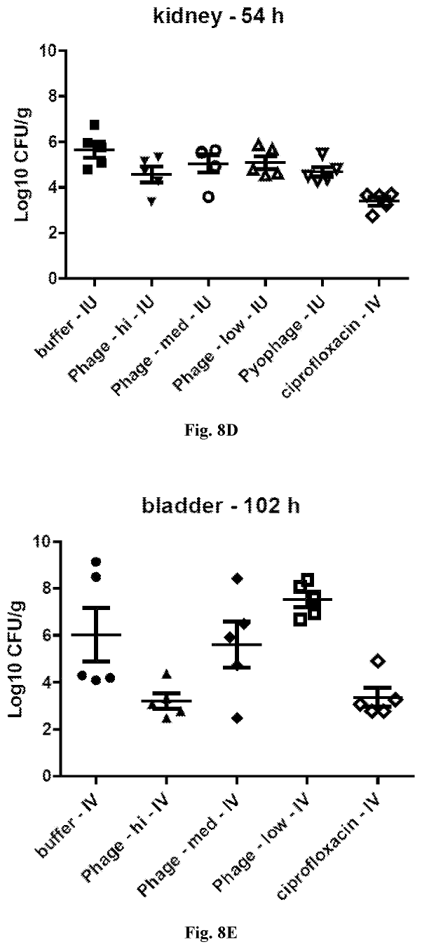

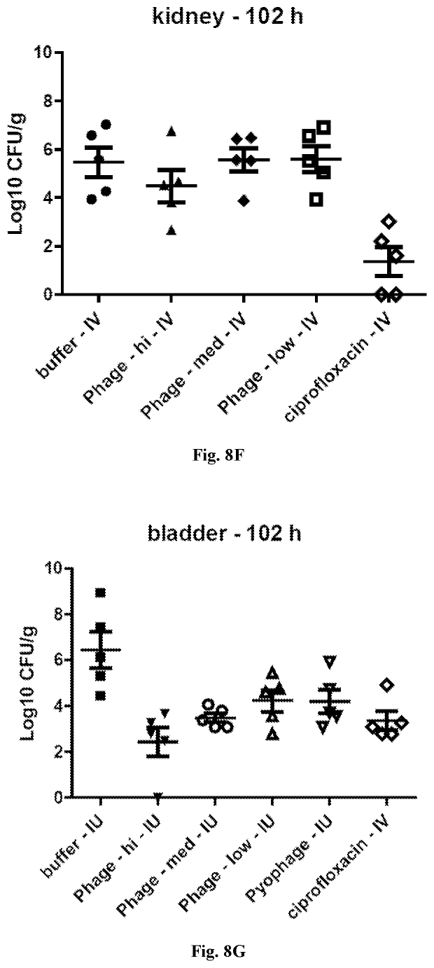

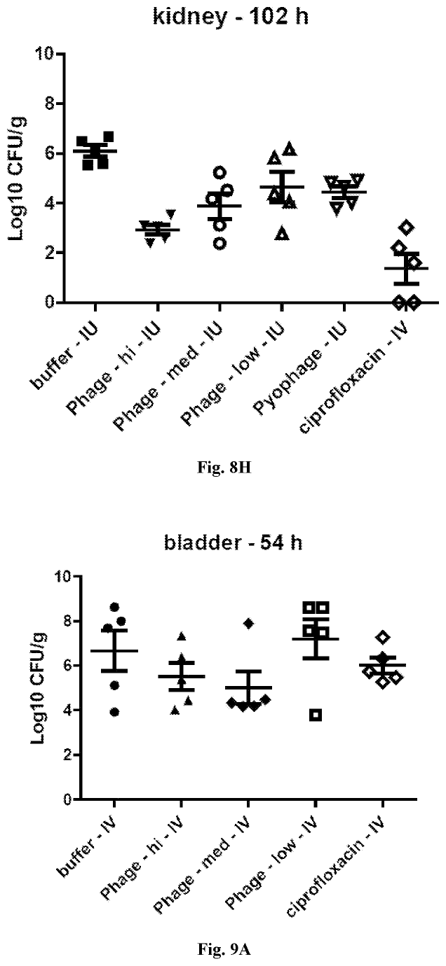

Bacterial density in the bladder and kidneys: The results are exemplified in FIG. 8A-FIG. 811, and Table 12. The density of bacteria in the bladder and kidneys at the start of treatment (48 h after infection) was 7.22±2.07 and 5.53±0.80 log₁₀ CFU/g tissue, respectively.

At 6 h after the first treatment (54 h after infection), only IV dosing with ciprofloxacin resulted in a significant reduction of bacteria in the bladder and kidneys compared to the buffer control treatment. Intraurethral (IU) dosing of the Locus phage cocktail at the high and medium dose levels and the Pyophage resulted in a significant reduction in bacteria in the bladder. None of the phage treatments reduced the bacteria in the kidneys.

At 6 h after the fifth treatment (102 h after infection), only the phage cocktail administered by IV injection at the high dose level significantly reduced bacterial density in the bladder. None of the phage treatments reduced bacterial levels in the kidneys when administered intravenously. When administered by the IU route, all three concentrations of the phage cocktail significantly reduced the bacteria in the bladder and the two higher concentrations reduced the bacteria in the kidneys. The Pyophage also significantly reduced bacteria in the bladder and kidneys.

TABLE 12
Density of E. coli (LFP 527) recovered from bladder and kidneys of infected mice
treated with TBS buffer, crPhage cocktail (Phage) 1 or Pyophage administered by IV
injection or IU instillation. Ciprofloxacin was administered by IV injection only.

| | | 54 h | | 102 h | |
|---|---|---|---|---|---|
| Grp | Treatment | bladder | kidney | bladder | kidney |
| 1 | Infected control-48 h | 7.22 ± 2.07 | 5.53 ± 0.80 | | |

IV

| | | | | | |
|---|---|---|---|---|---|
| 2 | Infected control-LR | 6.35 ± 0.40 | 5.22 ± 1.14 | 6.04 ± 2.54 | 5.47 ± 1.36 |
| 4 | Phage-high | 5.54 ± 0.83 | 5.20 ± 0.99 | 3.20 ± 0.72 * | 4.49 ± 1.49 |
| 6 | Phage-med | 5.68 ± 1.51 | 5.57 ± 1.15 | 5.61 ± 2.20 | 5.58 ± 1.05 |
| 8 | Phage-low | 8.26 ± 0.79 ** | 5.72 ± 0.71 | 7.53 ± 0.72 | 5.59 ± 1.19 |
| 11 | ciprofloxacin-IV | 3.90 ± 0.39 ** | 3.40 ± 0.41 * | 3.36 ± 0.89 | 1.36 ± 1.34 *** |

IU

| | | | | | |
|---|---|---|---|---|---|
| 3 | Infected control-LR | 7.92 ± 1.83 | 5.66 ± 0.77 | 6.45 ± 1.78 | 6.09 ± 0.52 |
| 5 | Phage-high | 2.40 ± 2.27 *** | 4.44 ± 0.79 | 2.43 ± 1.43 *** | 2.93 ± 0.45 *** |
| 7 | Phage-med | 4.52 ± 0.77 ** | 5.04 ± 0.86 | 3.48 ± 0.44 ** | 3.88 ± 1.13 ** |
| 9 | Phage-low | 6.51 ± 1.39 | 5.09 ± 0.63 | 4.23 ± 1.06 * | 4.64 ± 1.38 |
| 10 | Pyophage | 4.85 ± 0.85 ** | 4.69 ± 0.49 | 4.20 ± 1.14 * | 4.45 ± 0.43 * |
| 11 | ciprofloxacin-IV | 3.90 ± 0.39 *** | 3.40 ± 0.41 *** | 3.36 ± 0.89 ** | 1.36 ± 1.34 *** |

^{a} * = significant at P <0.05;
** = significant at P <0.01;
*** = significant at P <0.001

### Example 8. Evaluation of Phages Against E. coli in the Mouse UTI Model

The objective of this example was to evaluate the dose-response of phage cocktail against E. coli strain LFP 527 in the mouse UTI infection model. In addition, the virulence of three other E. coli strains was evaluated in the model.

Test phages: Buffer only, phage cocktail—High, Med, Low. The materials were stored refrigerated (2-8° C.) and were kept on ice during dosing. The crPhage cocktail was a mixture of phages p0033L-10 (ATCC No. PTA-126316), p004k-5 (ATCC No. PTA-126319), and p0071-16 (ATCC No. PTA-126320) at concentrations of 2×10¹¹ plaque forming units (PFU)/mL/phage (“high”), 2×10⁹ PFU/mL/phage (“medium”), or 2×10⁷ PFU/mL/phage (“low”). A 50 μl dose thus provided 1×10¹⁰ PFU/phage (“high”), 1×10⁸ PFU/phage (“medium”), or 1×10⁶ PFU/phage (“low”).

Standard: Ciprofloxacin hydrochloride (MP Biomedicals, LLC, Cat. No. 199020, Lot Q9860) was formulated in sterile saline at a concentration of 2.5 mg/mL.

Preparation of inoculum: Three strains were used in the study: Ec b1527, Ec b1533, and Ec b1557. These strains, along with Ec 527 (also referred to interchangeably as NC101 and Ec LFP527) were transferred to LB plates and incubated overnight 37° C. Colonies were transferred to 10 mL of LB broth in 250-mL Erlenmeyer flasks. Flasks were incubated statically at 37° C. for 18 h. After the first overnight growth, a 100-μL aliquot of the suspension from each flask was transferred to 25 mL of LB broth in 250-mL Erlenmeyer flasks. Flasks were incubated statically at 37° C. for 24 h. Each suspension was transferred to sterile conical tubes and centrifuged at 6,200 g for 5 min. The supernatants were decanted and the pellets resuspended in ˜2 mL of sterile PBS. The target concentration of the suspension was 1E+10 CFU/mL. The actual titer of each suspension was determined using the dilution plate count method and were as follows: b1527—8.0E+09, b1533—4.3E+09, b1557—5.5E+09, and Ec 527—8.8E+9 CFU/mL.

Animals: The female mice (Mus musculus), strain C3H/Hen, were obtained from Charles River Laboratories, Stone Ridge, N.Y. Mice were 56-days-old on the day of infection.

TABLE 13
Dosing regimen

| | | | Dose | Dose | No. |
|---|---|---|---|---|---|
| Grp | Treatment | Strain | route | (h after infection) | mice |
| 1 | Method dev.-48 h | Ec b1527 | NA | NA | 3 |
| 2 | Method dev.-48 h | Ec b1533 | NA | NA | 3 |
| 3 | Method dev.-48 h | Ec b1557 | NA | NA | 4 |
| 4 | Infected control-48 h | Ec 527 | NA | NA | 5 |
| 5 | Infected control | Ec 527 | IV | 48, 60, 72, 84 and 96 | 10 |
| 6 | | | IU | 48, 60, 72, 84 and 96 | 10 |
| 7 | Phage-high | Ec 527 | IV | 48, 60, 72, 84 and 96 | 10 |
| 8 | | | IU | 48, 60, 72, 84 and 96 | 10 |
| 9 | Phage-med | Ec 527 | IV | 48, 60, 72, 84 and 96 | 10 |
| 10 | | | IU | 48, 60, 72, 84 and 96 | 10 |
| 11 | Phage-low | Ec 527 | IV | 48, 60, 72, 84 and 96 | 10 |
| 12 | | | IU | 48, 60, 72, 84 and 96 | 10 |
| 13 | ciprofloxacin | Ec 527 | IV | 48, 60, 72, 84 and 96 | 10 |

Infection procedure: Each mouse was placed in an induction chamber filled with isoflurane carried in O₂ to initiate anesthesia. The mouse was then placed on an anesthesia board, ventral side up, with the nose inserted in a nosecone supplied with isoflurane. The lower abdomen was gently massaged to expel urine from the bladder. Using a 30 G×½ in. needle covered with polyethylene tubing (0.61 mm O.D.) and affixed to a 1 mL syringe, 50 μL of the inoculum was slowly injected into the bladder. After infection the mouse was returned to its cage.

Dosing procedure: Mice in Groups 5, 7, 9, 11 and 13 were dosed with 50 μL of the appropriate treatment by IV injection in a tail vein. Group 6, 8, 10 and 12 mice were dosed with 50 μL of each treatment by IU instillation. The instillation procedure for dosing was the same as that used for infection. Doses were administered at 48, 60, 72, 84 and 96 h after infection.

Tissue collection and processing: At 48 (Groups 1˜4 only), 54 and 102 h after infection, five mice per group were euthanized, the bladders and kidneys were removed and placed in sterile 13-mL flip-top tubes. The tubes were weighed and a volume of sterile PBS equivalent to 35× and 4× the weight of the bladders and kidneys, respectively, was added. The bladders were homogenized using the Tekmar Tissumizer and the kidneys using the Polytron 3100. Serial dilutions of the homogenates were plated on TSA and incubated at 37° C. overnight. Colony counts were used to calculate the CFU/g tissue.

Statistical analysis: One-way ANOVA with Dunnett's post-test comparing the buffer control to the phage treatments was performed on the log 10 CFU/g tissue data using GraphPad Prism version 5.04 for Windows, Graphpad Software, San Diego, Calif.

Method development strain results: The density of bacteria in the bladder and kidneys of EC LFP 527 at 48 h after infection was 5.22±2.21 and 5.66±0.36 log₁₀ CFU/g tissue, respectively (Table 15). No colonies were recovered from the bladders of the three method development strains. Recoveries from the kidneys was lower than for Ec LFP 527, with densities ranging from 2.67 to 4.59 log₁₀ CFU/g tissue.

TABLE 14
Density of E. coli strains recovered from bladder and kidneys of
infected mice at 48 h after infection.

| | 48 h | |
|---|---|---|
| Strain | bladder | kidney |
| Ec b1527 | 0 | 4.59 ± 0.56 |
| Ec b1533 | 0 | 2.70 ± 0.83 |
| Ec b1557 | 0 | 2.67 ± 0.79 |
| Ec 527 | 5.22 ± 2.21 | 5.66 ± 0.36 |

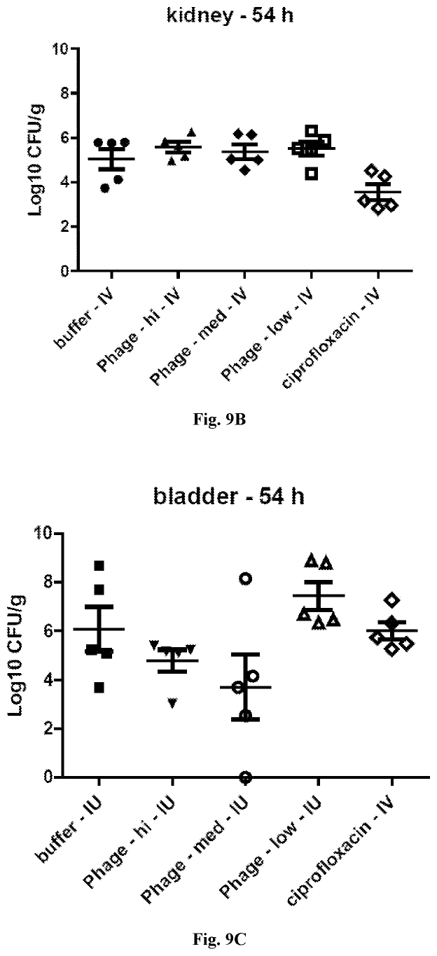

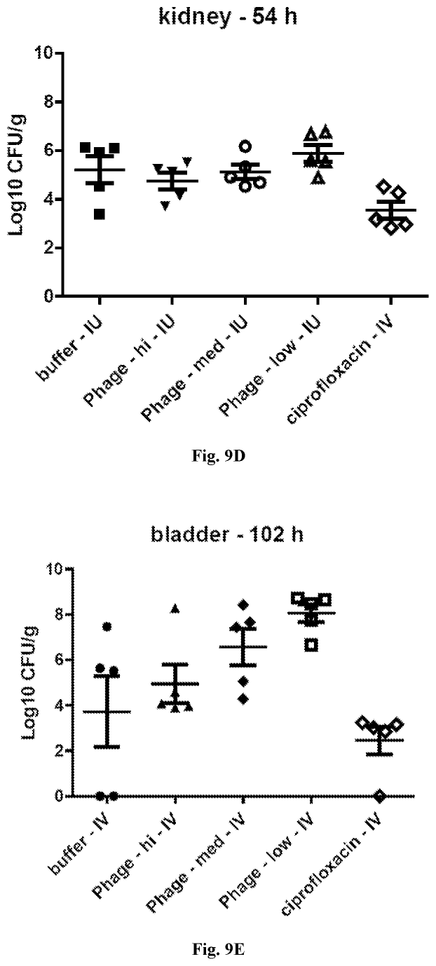

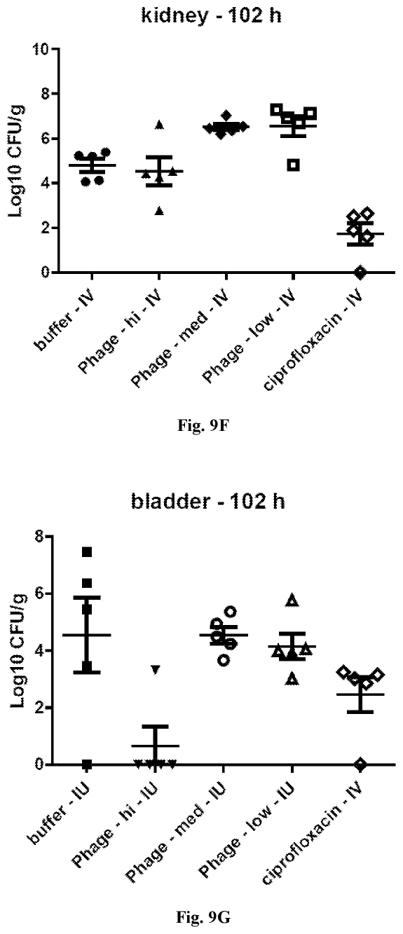

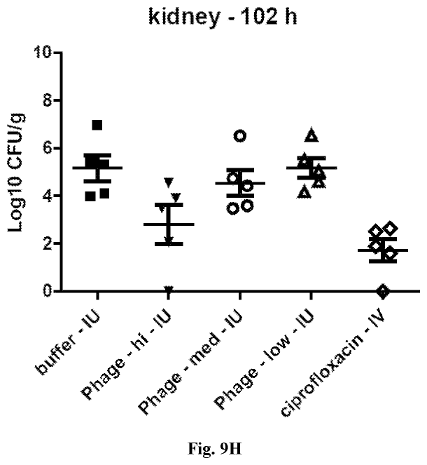

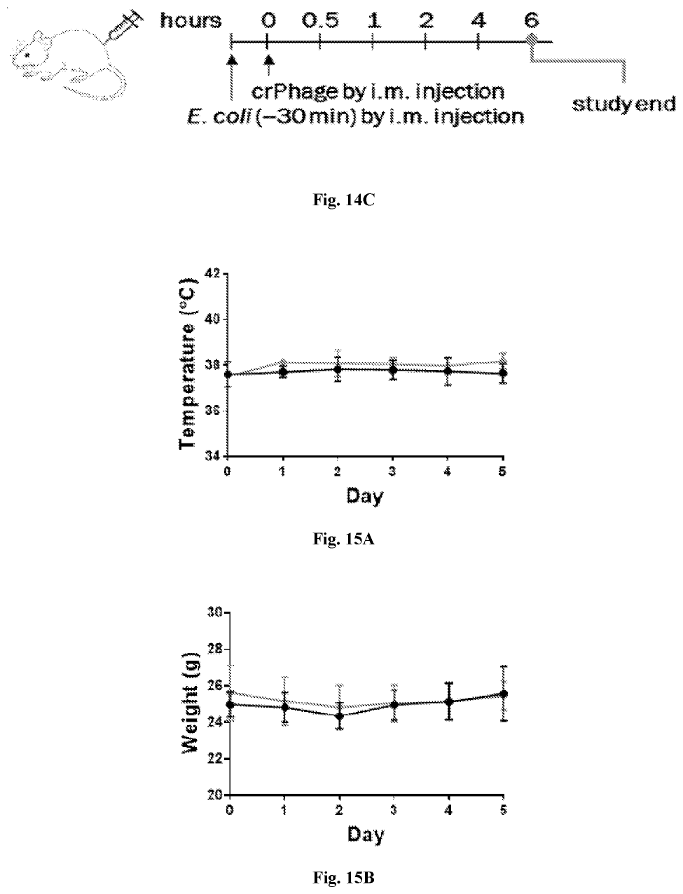

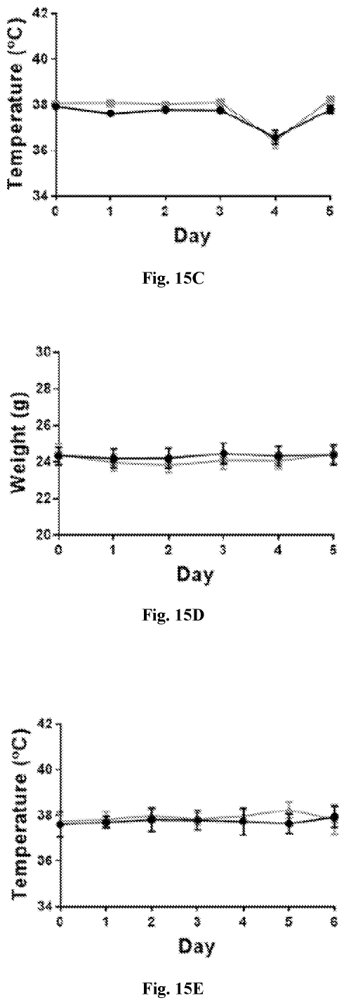

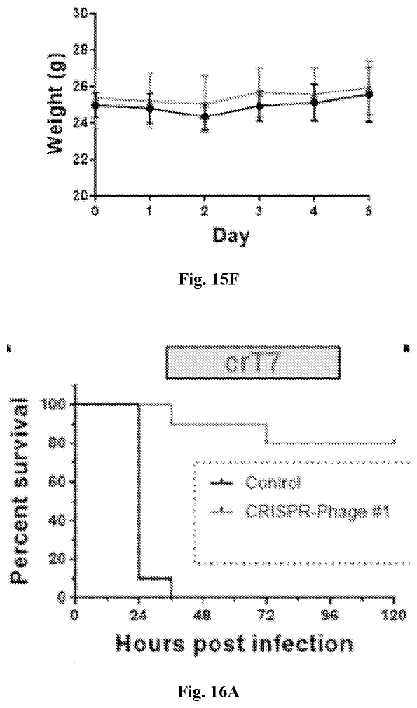

Phage treatment results: At 6 h after the first treatment (54 h after infection) none of the treatments administered by either route reduced bacterial density in the bladder and only ciprofloxacin significantly reduced the density in the kidneys (FIG. 15A-FIG. 9D, Table 15).

At 6 h after the fifth treatment (102 h after infection), the high dose of phage administered IU significantly reduced bacterial density in the kidney and bladders. None of the phage treatments reduced bacterial density when administered intravenously (IV). Ciprofloxacin administered IV significantly reduced bacterial density in the kidneys (FIG. 9E-FIG. 911, Table 15).

TABLE 15
Density of E. coli (LFP 527) recovered from bladder and kidneys of infected
mice treated with vehicle or Locus phage cocktail by IV injection or
IU instillation. Ciprofloxacin was administered by IV injection only.

| | | 54 h | | 102 h | |
|---|---|---|---|---|---|
| Grp | Treatment | bladder | kidney | bladder | kidney |

IV

| | | | | | |
|---|---|---|---|---|---|
| 5 | Vehicle control | 6.67 ± 2.03 | 5.03 ± 1.03 | 3.73 ± 3.49 | 4.80 ± 0.65 |
| 7 | Phage-high | 5.51 ± 1.36 | 5.57 ± 0.51 | 4.96 ± 1.88 | 4.53 ± 1.38 |
| 9 | Phage-med | 5.01 ± 1.61 | 5.37 ± 0.74 | 6.57 ± 1.80 | 6.51 ± 0.32 |
| 11 | Phage-low | 7.21 ± 1.98 | 5.50 ± 0.71 | 8.05 ± 0.88 | 6.56 ± 1.01 |
| 13 | Ciprofloxacin-IV | 6.01 ± 0.80 | 3.54 ± 0.78 * | 2.45 ± 1.38 | 1.73 ± 1.06 *** |

IU

| | | | | | |
|---|---|---|---|---|---|
| 6 | Vehicle control | 6.08 ± 2.05 | 5.21 ± 1.22 | 4.55 ± 2.94 | 5.17 ± 1.21 |
| 8 | Phage-high | 4.78 ± 0.99 | 4.74 ± 0.77 | 0.66 ± 1.49 ** | 2.81 ± 0.32 * |
| 10 | Phage-med | 3.71 ± 2.96 | 5.12 ± 0.66 | 4.54 ± 0.65 | 4.55 ± 1.22 |
| 12 | Phage-low | 7.43 ± 1.29 | 5.89 ± 0.80 | 4.14 ± 0.99 | 5.18 ± 0.91 |
| 13 | Ciprofloxacin-IV | 6.01 ± 0.80 | 3.54 ± 0.78 * | 2.45 ± 1.38 | 1.73 ± 1.06 ** |

^{a} * = significant at P <0.05;
** = significant at P <0.01;
*** = significant at P <0.001

### Example 9. Evaluation of Phages Against E. coli in the Mouse UTI Model—Pooled Results from Example 7 and 8

The objective of these studies was to evaluate the engineered bacteriophage (crPhage) cocktail against Escherichia coli (E. coli) strain LFP 527 in the mouse urinary tract infection (UTI) model.

Proof-of-concept clustered regularly interspaced short palindromic repeats (CRISPR)-enhanced phage product against E. coli was developed as a cocktail of up to three distinct obligate lytic bacteriophages that contain an identical deoxyribonucleic acid (DNA) sequence encoding a functional self-targeting CRISPR ribonucleic acid (crRNA) (hereafter termed ‘crRNA cassette’) embedded in the wild-type phage genome. Bacteriophages were engineered by homologous recombination in E. coli cells with active phage infection.

Each phage was engineered with a similar crRNA cassette that contained two elements: (1) a leuO transcription factor gene derived from E. coli downstream of a synthetic promoter and (2) a repeat-spacer-repeat encoding a crRNA targeting the ftsA gene downstream of a synthetic promoter. Upon DNA transduction during infection, leuO would be expressed from the phage genome and subsequently upregulate expression of the endogenous Type I-E CRISPR-Cas3 operon in E. coli. Concurrently, the synthetic ftsA-targeting crRNA would be expressed from the phage genome that is recognized and processed by the endogenous Type I-E CRISPR-Cas3 protein complex. This crRNA is then loaded onto a CRISPR-Cas3 complex and thereby directs the targeting and degradation of target bacterial DNA.

The crPhage cocktail was a mixture of phages p0033L-10 (ATCC No. PTA-126316), p004k-5 (ATCC No. PTA-126319), and p0071-16 (ATCC No. PTA-126320) at concentrations of 2×10¹¹ plaque forming units (PFU)/mL/phage (“high”), 2×10⁹ PFU/mL/phage (“medium”), or 2×10⁷ PFU/mL/phage (“low”). A 50 μl dose thus provided 1×10¹⁰ PFU/phage (“high”), 1×10⁸ PFU/phage (“medium”), or 1×10⁶ PFU/phage (“low”). Lactated Ringer's (LR) solution served as a negative control and ciprofloxacin (Cipro) served as a positive control.

Female C3H/HeN mice were infected with E. coli strain LFP 527 via intraurethral (IU) instillation. Beginning 48 hours after infection, a test article of phage cocktail containing p0033L-10, p004k-5, and p0071-16 was administered at time zero and every 12 hours for 48 hours (for a total of 5 doses). Phage cocktail with a range of phage concentrations (Table 16) or vehicle was delivered either intravenously (IV) or by IU instillation into the bladder. The phage cocktails were prepared using LR.

TABLE 16
Group assignment and dose levels

| | No. of | | | | |
|---|---|---|---|---|---|
| | Animals | | | | Total |
| Dose | per | | Dose | Dose (h after | dose per |
| Group | study | Test Article | Route | infection ^{a}) | phage |
| 1 | 5 | N/A | N/A | N/A | N/A |
| 2 | 10 | Lactated Ringer's | IV | 48, 60, 72, 84 and 96 | N/A |
| 3 | 10 | Lactated Ringer's | IU | 48, 60, 72, 84 and 96 | N/A |
| 4 | 10 | crPhage cocktail | IV | 48, 60, 72, 84 and 96 | 1 × 10¹⁰ PFU |
| 5 | 10 | crPhage cocktail | IU | 48, 60, 72, 84 and 96 | 1 × 10¹⁰ PFU |
| 6 | 10 | crPhage cocktail | IV | 48, 60, 72, 84 and 96 | 1 × 10⁵ PFU |
| 7 | 10 | crPhage cocktail | IU | 48, 60, 72, 84 and 96 | 1 × 10⁶ PFU |
| 8 | 10 | crPhage cocktail | IV | 48, 60, 72, 84 and 96 | 1 × 10⁶ PFU |
| 9 | 10 | crPhage cocktail | IU | 48, 60, 72, 84 and 96 | 1 × 10⁶ PFU |
| 10 ^{b} | 10 | PyoPhage | IU | 48, 60, 72, 84 and 96 | N.D. |
| 11 | 10 | Ciprofloxacin | IV | 48, 60, 72, 84 and 96 | 5 mg/kg |

IU = intraurethral;
IV = intravenous(ly);
N/A = not applicable;
N.D. = not determined;
PFU = plaque forming units
^{a} Testing was performed 6 hours post administration of crPhage cocktail at each time point.
^{b} PyoPhage was only used in Study 037192.

The following steps were performed to accomplish PFU enumeration:
Tissue homogenates and blood samples were centrifuged for 10 minutes at 4000×g.
Tissue homogenates were additionally filtered through 0.45 μm filters to remove bacteria.
Tissue homogenate filtrates, blood sample supernatants, and unprocessed urine samples were then serially diluted in 1× tris-buffered saline (TBS)+10 mM MgCl₂+10 mM CaCl₂.
2 μl of each dilution were spotted onto soft agar overlays of phage-specific bacterial indicator strains, allowed to dry, and incubated overnight at 37° C.
The following day, plaques were counted and the titer of each phage in the undiluted sample was determined.

Density of E. coli (CFU/g): Significant effects were observed in the bladder following IU delivery for high dose (1×10¹⁰ PFU) crPhage after 54 hours (i.e., 6 hours after the administration of crPhage at the 48-hour time point; a 2.90-log reduction when compared with LR [p<0.0001]) and after 102 hours (ie, 6 hours after the administration of crPhage at the 96-hour time point; a 2.94-log reduction when compared with LR [p<0.0001]). Medium (1×10⁸ PFU) and low (1×10⁶ PFU) doses produced 2.63-log (p<0.0001) and −0.03-log reductions, respectively, after 54 hours, and 1.74 and 1.56 reductions, respectively, after 102 hours. A significant effect was also observed in the kidney 102 hours after IU delivery for high dose crPhage (a 2.60-log reduction when compared with LR [p=0.0002]). Following IU delivery, observed CFUs in the kidney and the bladder were similar following treatment with high dose crPhage and Cipro.

Density of crPhage (PFU/g tissue): Following IU delivery of high, medium, and low doses of crPhage to infected mice, all 3 dose levels resulted in detectable phage in the bladder 54 hours after IU administration, with greater numbers of PFUs/g tissue detected for the medium and low doses. After 102 hours, all 3 doses had further reductions in PFUs but remained above the limit of detection. In the kidney, phages were detectable after 54 hours and after 102 hours, with reductions in PFUs/g tissue across doses over time. Phages were not detected in the blood at 54 or 102 hours after IU administration; however, high levels (which were similar across dose groups) were observed in the urine at 78 hours post administration.

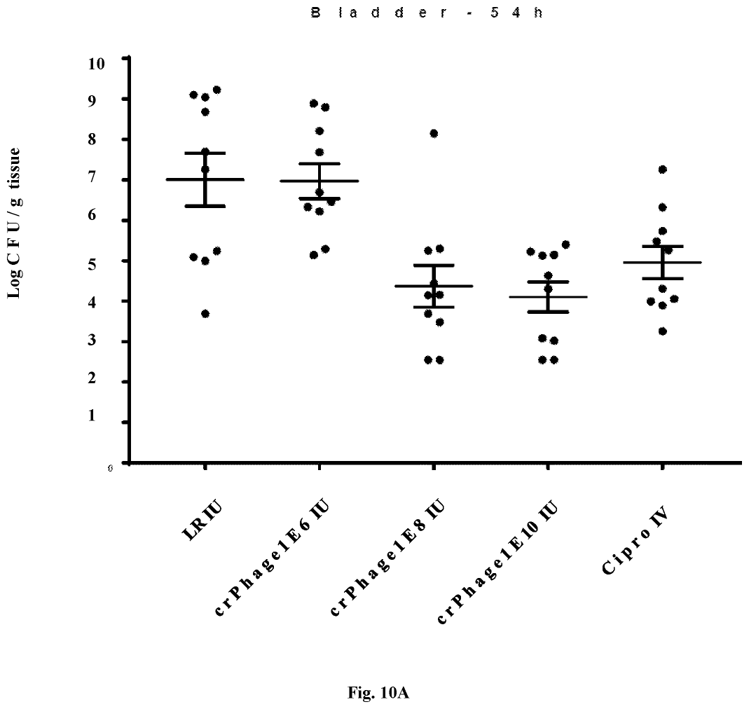

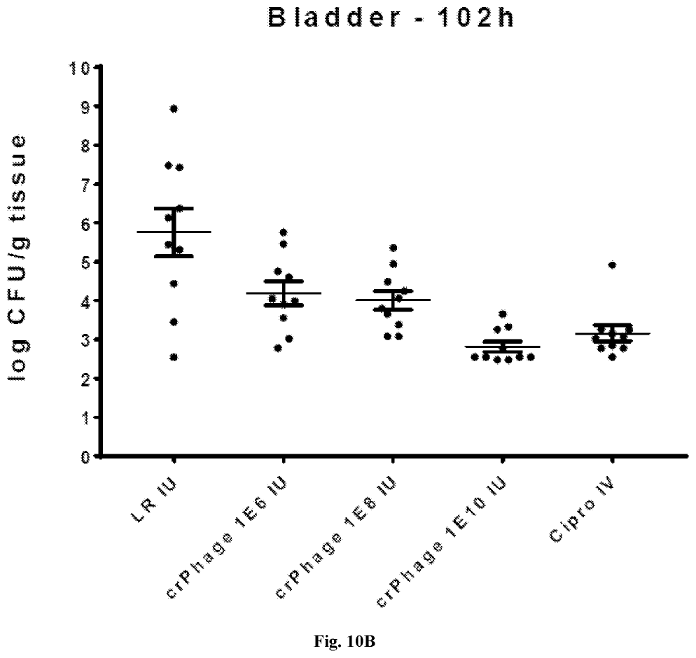

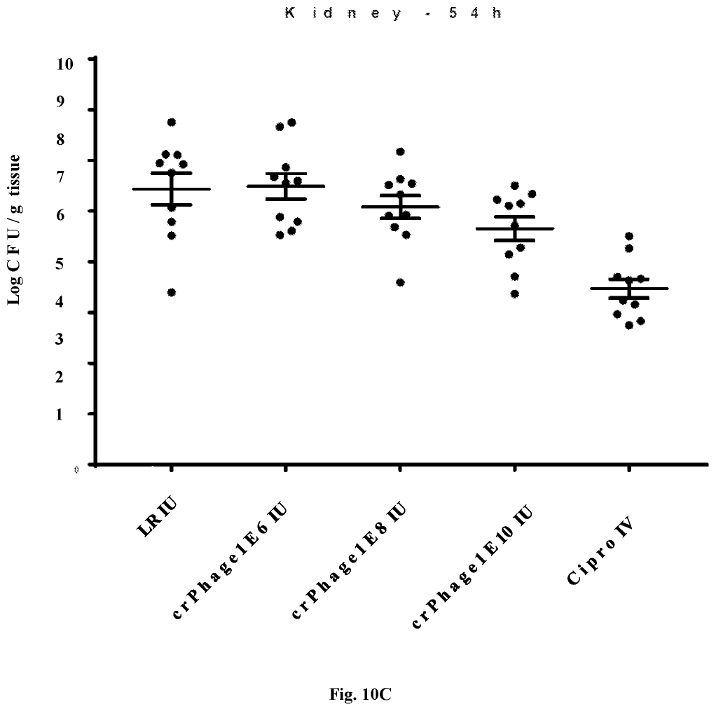

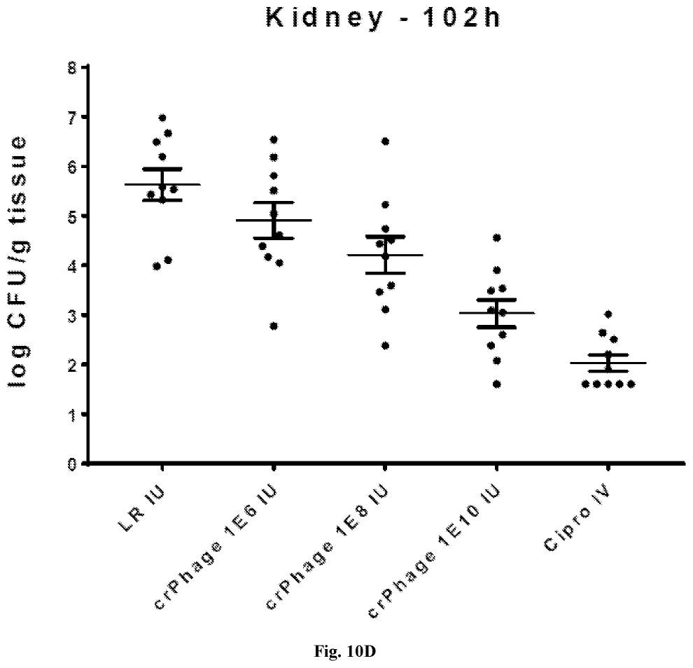

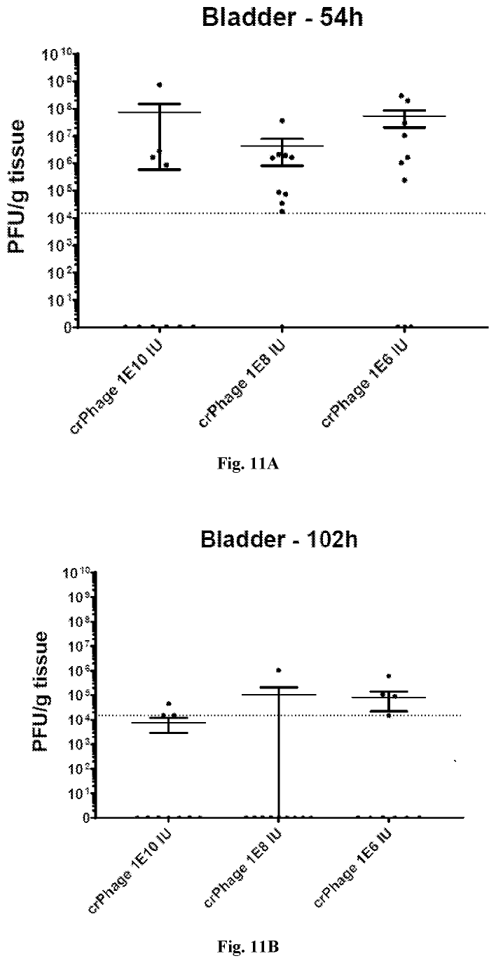

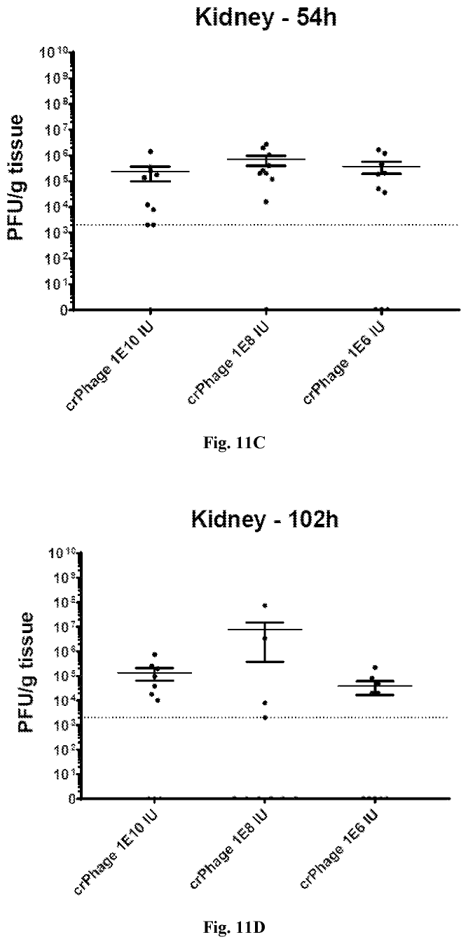

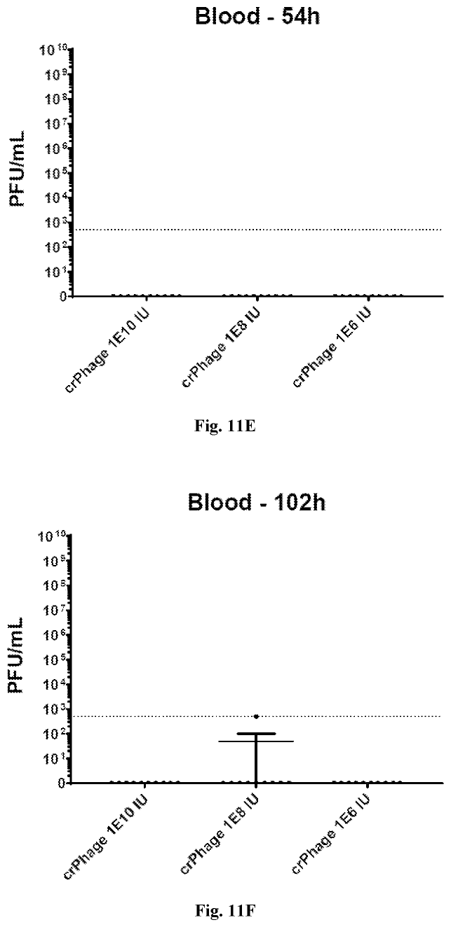

After IU administration, crPhage was detectable in the bladder and kidney for all dose groups up to 102 hours post-administration. No crPhage was detectable in the blood, and clearance via urine was high at 78 hours. Pooled results are exemplified in FIG. 10A-FIG. 10D, FIG. 11A-FIG. 11G, Table 17, and Table 18.

TABLE 17
Bladder and kidney CFUs after IU treatment.

| | | | | | |
|---|---|---|---|---|---|
| | # samples | Mean log | | Delta from LR | Delta from Cipro |
| Treatment | <LOD | CFU/g | SEM | (significance) | (significance) |

Bladder, 54 hours after IU treatment

| | | | | | |
|---|---|---|---|---|---|
| Ciprofloxacin^{a} | 0 | 4.96 | 0.40 | −1.55 (n.s.) | N.A. |
| Lactated Ringer's | 0 | 7.00 | 0.66 | N.A. | N.A. |
| crPhage 1E6 | 0 | 6.97 | 0.43 | −0.03 (n.s.) | +2.01 (p = 0.0065) |
| crPhage 1E8 | 1 | 4.37 | 0.52 | −2.63 (p <0.0001) | −0.59 (n.s.) |
| crPhage 1E10 | 2 | 4.10 | 0.37 | −2.90 (p <0.0001) | −0.86 (n.s.) |

Bladder, 102 hours after IU treatment

| | | | | | |
|---|---|---|---|---|---|
| Ciprofloxacin^{a} | 1 | 3.16 | 0.21 | −2.23 (p = 0.0033) | N.A. |
| Lactated Ringer's | 1 | 5.75 | 0.62 | N.A. | N.A. |
| crPhage 1E6 | 0 | 4.19 | 0.31 | −1.56 (n.s.) | +1.06 (n.s.) |
| crPhage 1E8 | 0 | 4.01 | 0.24 | −1.74 (n.s.) | +0.85 (n.s.) |
| crPhage 1E10 | 5 | 2.81 | 0.14 | −2.94 (p <0.0001) | −0.35 (n.s.) |

Kidney, 54 hours after IU treatment

| | | | | | |
|---|---|---|---|---|---|
| Ciprofloxacin^{a} | 0 | 3.47 | 0.19 | −1.66 (n.s.) | N.A. |
| Lactated Ringer's | 0 | 5.44 | 0.31 | N.A. | N.A. |
| crPhage 1E6 | 0 | 5.49 | 0.25 | +0.05 (n.s.) | +2.02 (p = 0.0063) |
| crPhage 1E8 | 0 | 5.08 | 0.23 | −0.36 (n.s.) | +1.61 (n.s.) |
| crPhage 1E10 | 0 | 4.65 | 0.23 | −0.79 (n.s.) | +1.18 (n.s.) |

Kidney, 102 hours after IU treatment

| | | | | | |
|---|---|---|---|---|---|
| Ciprofloxacin^{a} | 3 | 2.03 | 0.17 | −3.11 (p <0.0001) | N.A. |
| Lactated Ringer's | 0 | 5.63 | 0.32 | N.A. | N.A. |
| crPhage 1E6 | 0 | 4.91 | 0.36 | −0.72 (n.s.) | +2.88 (p <0.0001) |
| crPhage 1E8 | 0 | 4.21 | 0.37 | −1.42 (n.s.) | +2.18 (p = 0.0044) |
| crPhage 1E10 | 1 | 3.03 | 0.28 | −2.60 (p = 0.0002) | +1.00 (n.s.) |

ANOVA = analysis of variance;
Cipro = ciprofloxacin;
CFU = colony forming units;
IU = intraurethral;
LOD = limit of detection;
LR = lactated ringer's solution;
N.A. = not applicable;
n.s. not significant;
SEM = standard error of the mean
^{a}Ciprofloxacin was administered intravenously and is compared with intravenous LR.
Note:
Samples that did not produce CFUs were considered to be at the limit of detection (kidney LOD = 40 CFU/g; bladder LOD = 300 CFU/g for study 037192 and 350 CFU/g for study 037204). Statistics were performed by doing a two-way ANOVA of log-transformed data. The p-value from the ANOVA is reported.

TABLE 18
Bladder, Kidney, Blood, and Urine PFUs after IU treatment

| | | | |
|---|---|---|---|
| | | Mean log PFU/g | |
| Treatment | # samples <LOD | or PFU/mL | SEM |

Bladder, 54 hours after IU treatment

| | | | |
|---|---|---|---|
| crPhage 1E6 | 3 | 6.14 | 0.53 |
| crPhage 1E8 | 1 | 5.54 | 0.36 |
| crPhage 1E10 | 6 | 5.26 | 0.50 |

Bladder, 102 hours after IU treatment

| | | | |
|---|---|---|---|
| crPhage 1E6 | 6 | 4.52 | 0.17 |
| crPhage 1E8 | 9 | 4.39 | 0.18 |
| crPhage 1E10 | 7 | 4.26 | 0.05 |

Kidney, 54 hours after IU treatment

| | | | |
|---|---|---|---|
| crPhage 1E6 | 3 | 4.77 | 0.36 |
| crPhage 1E8 | 1 | 5.30 | 0.30 |
| crPhage 1E10 | 1 | 4.54 | 0.34 |

Kidney, 102 hours after IU treatment

| | | | |
|---|---|---|---|
| crPhage 1E6 | 5 | 4.00 | 0.25 |
| crPhage 1E8 | 6 | 4.14 | 0.52 |
| crPhage 1E10 | 3 | 4.43 | 0.30 |

Blood, 54 hours after IU treatment

| | | | |
|---|---|---|---|
| crPhage 1E6 | 10 | <LOD | 0.00 |
| crPhage 1E8 | 10 | <LOD | 0.00 |
| crPhage 1E10 | 10 | <LOD | 0.00 |

Blood, 102 hours after IU treatment

| | | | |
|---|---|---|---|
| crPhage 1E6 | 10 | <LOD | 0 00 |
| crPhage 1E8 | 9 | 2.70 | 0.00 |
| crPhage 1E10 | 10 | <LOD | 0.00 |

Urine, 78 hours after IU treatment

| | | | |
|---|---|---|---|
| crPhage 1E6 | 2 | 6.38 | 0.76 |
| crPhage 1E8 | 0 | 7.43 | 0.28 |
| crPhage 1E10 | 0 | 7.77 | 0.18 |

IU = intraurethral;
LOD = limit of detection;
PFU = plaque-forming unit;
SEM = standard error of the mean
Note:
Samples that did not produce PFUs were considered to be at the limit of detection (kidney LOD = 2000 PFU/g or 3.3 log PFU/g; bladder LOD = 15,000 PFU/g or 4.18 log PFU/g for study 037192 and 17,500 PFU/g or 4.24 log PFU/g for study 037204; blood LOD = 500 PFU/mL or 2.70 log PFU/mL; urine LOD = 500 PFU/mL or 2.70 log PFU/mL).

### Example 11. Tolerability of Experimental Phages in Female Mice

The objective of this study was to determine the tolerability of four experimental phage treatments, crT7M (3.7×10⁹ PFU/dose/phage), crT4 (1.4×10⁷ PFU/dose/phage), crT7 (1.1×10⁸ PFU/dose/phage), and a crPhage cocktail (1.3×10⁹ PFU/dose/phage) containing a mixture of all phages, in female mice.

Group 1 mice were dosed, by IP injection, with saline vehicle and mice in Groups 2-5 mice were dosed five times, by IP injection, with 1× phage at 12-hour intervals.

Body temperatures were measured using a Physitemp® digital thermometer equipped with a rectal probe. A small amount of lubricating jelly was placed on the tip of the probe and the probe was inserted into the rectum of each mouse. Once the temperature reading stabilized the reading was entered into the data collection system.

TABLE 19
Dosing Regimen

| Dose | Number of | | Dose | Dose Volume | Dose schedule |
|---|---|---|---|---|---|
| Group | Animals | Test Article | Route | (mL/animal) | (hours) |
| 1 | 5 | Saline | IP | 0.1 | 0, 12, 24, 36, 48 |
| 2 | 5 | crT7M | IP | 0.1 | 0, 12, 24, 36, 48 |
| 3 | 5 | crT4 | IP | 0.1 | 0, 12, 24, 36, 48 |
| 4 | 5 | crT7 | IP | 0.1 | 0, 12, 24, 36, 48 |
| 5 | 5 | crT phage | IP | 0.1 | 0, 12, 24, 36, 48 |
| | | cocktail | | | |

IP = intraperitoneal
Note:
crT cocktail = cocktail of crT7M, crT4, and crT7 phages.

Mortality: All mice in all of the groups survived through the end of the study.

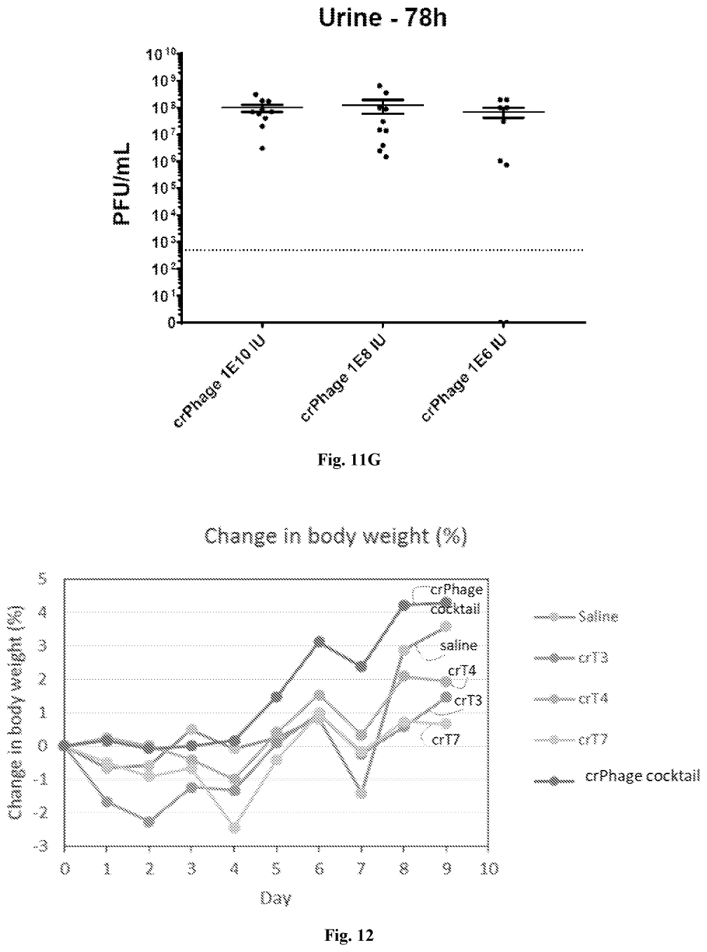

Body weight (Table 20, FIG. 12). The group of mice dosed once with crT7M lost 1.2-2.3% of their starting weight during dosing. By Day 5 the mean body weight increased to near the starting weight and then increased to +1.5% by the end of the study. The mean body weight of mice dosed with crT4 was relatively constant during dosing with a maximum loss of 1% on Day 4. The mean body weight then increased to +1.9% by the end of the study. Similar to crT4, body weights of mice dosed with crT7 remained relatively constant during dosing. On Day 4 the mean body weight was 2.4% lower than the starting weight, but was then +0.7% higher by the end of the study. Mice dosed with the phage cocktail exhibited the least amount of weight loss during dosing and then gained the most weight (+4.3%) by the end of the study.

TABLE 20
Mean body weight (g) and percent weight difference from Day 1 (prior to
dosing).

| | | Pre- | Day of study | | | | | | | | |
|---|---|---|---|---|---|---|---|---|---|---|---|
| Grp | Treatment | dose | 1 | 2 | 3 | 4 | 5 | 6 | 7 | 8 | 9 |
| s | Saline | 24.3 | 24.2 | 24.2 | 24.5 | 24.3 | 24.4 | 24.5 | 24.0 | 25.1 | 25.2 |
| 3 | 7 | 24.4 | 24.0 | 23.8 | 24.1 | 24.1 | 24.4 | 24.6 | 24.3 | 24.5 | 24.7 |
| a | aT4 | 24.5 | 24.5 | 24.5 | 24.4 | 24.2 | 24.6 | 24.8 | 24.5 | 25.0 | 24.9 |
| 4 | e | 24.3 | 24.2 | 24.1 | 24.2 | 23.9 | 24.2 | 24.6 | 24.3 | 24.5 | 248.5 |
| 5 | | 25.5 | 25.5 | 25.0 | 25.6 | 25.5 | 26.5 | 26.3 | 26.1 | 26.6 | 26.6 |

% weight difference from Day 1

| | | | | | | | | | | | |
|---|---|---|---|---|---|---|---|---|---|---|---|
| 1 | Saline | — | −0.7 | −0.6 | 0.5 | −0.1 | 0.2 | 0.8 | −1.4 | 2.9 | 3.6 |
| 2 | crTTM | — | −1.7 | −2.3 | −1.2 | −1.3 | 0.1 | 1.0 | −0.2 | 0.6 | 1.5 |
| 3 | crT4 | — | 0.2 | 0.0 | −0.4 | −1.0 | 0.4 | 1.5 | −0.3 | 2.1 | 1.9 |
| 4 | crT7 | — | −0.5 | −0.9 | −0.7 | −2.4 | −0.4 | 1.0 | −0.2 | 0.7 | 0.7 |
| 5 | crT Phage Cocktall | | 0.2 | −0.1 | 0.0 | 0.2 | 1.5 | 3.1 | 2.4 | 4.2 | 4.3 |

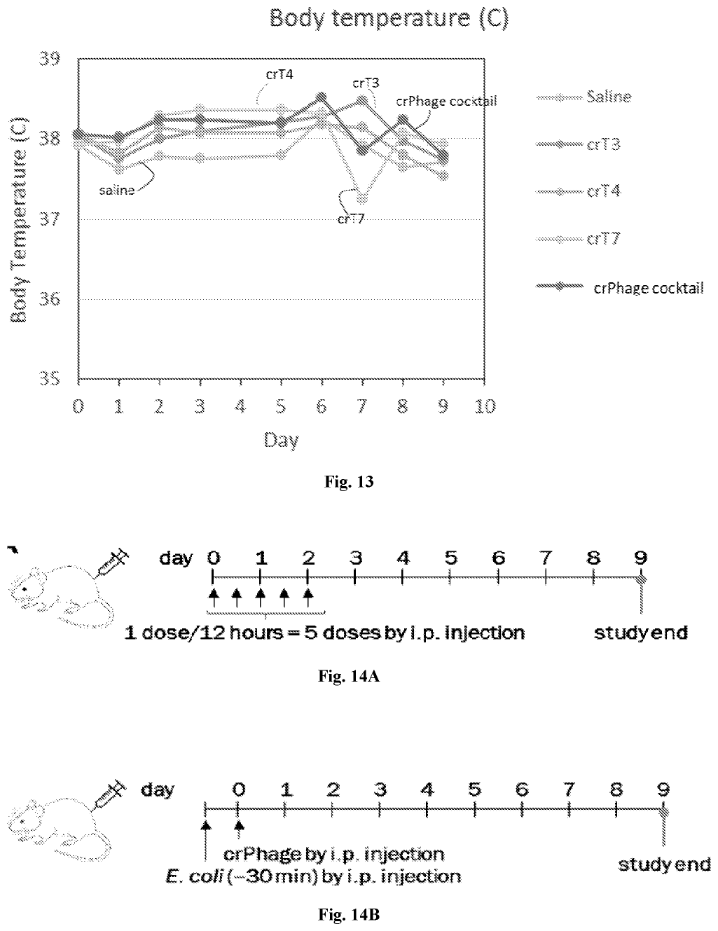

Body temperature results are exemplified in Table 21, FIG. 13. Body temperatures remained fairly constant over the course of the study. Mean temperatures of the phage-treated groups were within one degree of the saline-treated mice at each time point.

TABLE 21
Mean body temperatures (° C.) of mice treated with saline, crT7M, crT4, crT7 or
crPhage Cocktail.

| | | Pre- | Day of study | | | | | | | | |
|---|---|---|---|---|---|---|---|---|---|---|---|
| Grp | Treatmentr | dose | 1 | 2 | 3 | 4^{a} | 5 | 6 | 7 | 8 | 9 |
| 1 | Saine | 37.9 | 37.6 | 37.8 | 37.8 | 36.6 | 38.3 | 37.8 | 37.9 | 37.6 | 37.7 |
| 2 | crT7M | 38.0 | 38.1 | 38.0 | 38.1 | 36.4 | 38.2 | 38.3 | 38.5 | 38.0 | 37.7 |
| 3 | crT4 | 38.1 | 38.1 | 38.1 | 38.1 | 36.5 | 38.1 | 38.2 | 38.1 | 37.8 | 37.5\| |
| 4 | erT7 | 37.9 | 38.2 | 38.3 | 38.4 | 37.1 | 38.4 | 38.3 | 37.2 | 38.1 | 37.9 |
| 5 | crPhage Cocktan | 38.1 | 38.0 | 38.2 | 38.2 | 37.2 | 38.2 | 38.5 | 37.9 | 38.2 | 37.8 |

### Example 12. In Vivo Studies

As a standard set of proof-of-concept studies in vivo, a series of animal studies were conducted to test the tolerability of a crPhage formulation (FIG. 14A), the ability to rescue mice in a peritonitis model (FIG. 14B) and the quantitative measurement of in vivo bioburden reduction (FIG. 14C).

Prior to survival studies in the peritonitis model, the tolerability of each crPhage was determined in vivo. In the tolerability study, each mouse received one 100 μL dose per day by intraperitoneal (IP) injection for 5 days with 2.0×10¹¹ PFU/day/mouse of crT7, 5 days with 3.7×10⁹ PFU/day/mouse of crT7M or 1 day with 6.0×10⁸ PFU/day/mouse of crT4. crT7 and crT7M were suspended in sterile, endotoxin-free 0.9% saline, while crT4 was suspended in sterile, endotoxin-free 1× tris-buffered saline (pH 7.4) supplemented with 10 mM of each CaCl₂ and MgCl₂. No overt toxicity was observed during veterinary observation and no measurable changes in body temperature or body weight were noted after dosing with each crPhage preparation (FIG. 15A-FIG. 15F).

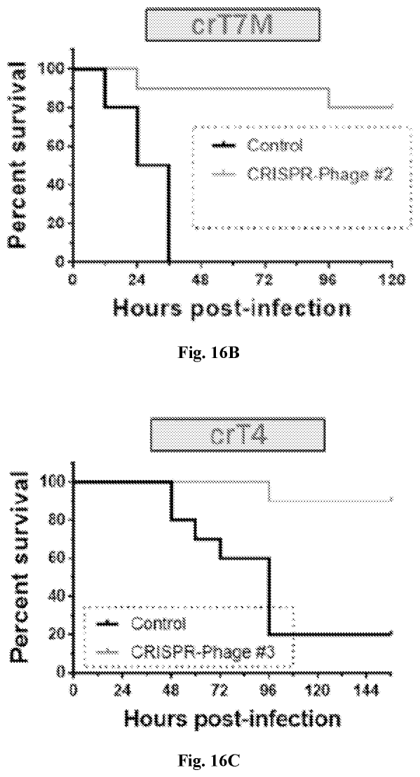

Based on the activity of 3 crPhages against a fecal isolate (ATCC 8739, crT7M and crT7) or lab strain (MG1655, crT4), as well as successful tolerability at the highest tested doses (FIG. 16A-FIG. 16C), the 3 crPhages were evaluated in a murine peritonitis model using E. coli. Mice were injected IP with a lethal dose of E. coli followed within 30 minutes by IP injections of saline or crPhages (FIG. 16A-FIG. 16C). crT7 and crT7M were suspended in sterile, endotoxin-free 0.9% saline, while crT4 was suspended in sterile, endotoxin-free 1× tris-buffered saline (pH 7.4) supplemented with 10 mM each CaCl₂ and MgCl₂. Single-dose administration of crPhage (2.0×10¹¹ PFU/dose of crT7, 3.7×10⁹ PFU/dose of crT7M or 6.0×10⁸ PFU/dose of crT4) resulted in significant protection in this acute, highly lethal bacterial challenge (FIG. 16A-FIG. 16C). These data demonstrate that bacteriophages are able to infect and kill sufficient numbers of target bacteria.

The original crPhage stocks used in this study have a potency of 2×10¹² PFU/mL of crT7, 4×10¹² PFU/mL of crT7M, and 2×10¹¹ PFU/mL of crT4 with each phage suspended in sterile, endotoxin-free 1× tris-buffered saline (pH 7.4). The 3 crPhages were pooled into a cocktail with a final concentration of 1×10¹¹ PFU/mL of each phage containing an estimated endotoxin content of <10³ EU/mL.

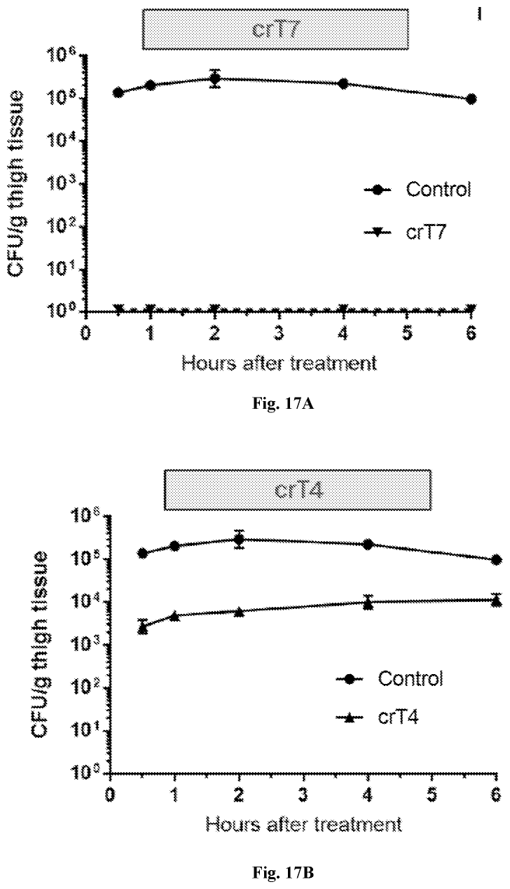

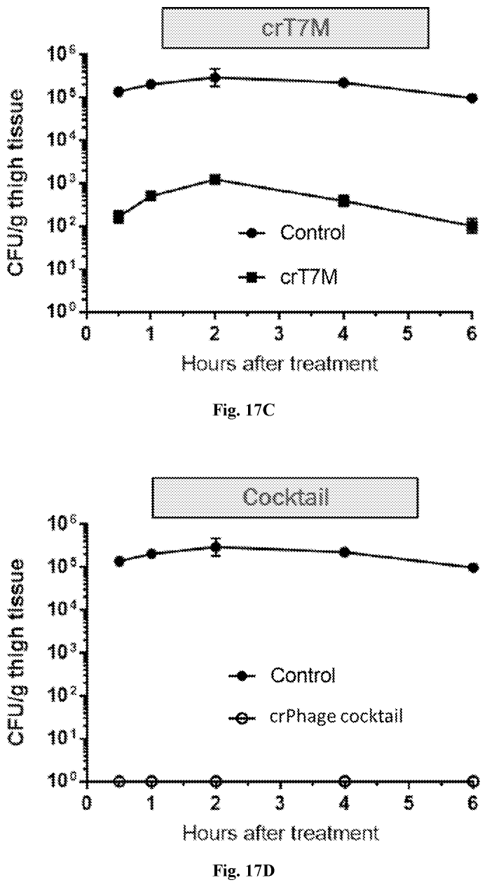

As another demonstration of in vivo efficacy, a thigh infection model in mice was conducted to measure bioburden reduction after crPhage treatment. One and 4 days prior to bacterial inoculation, mice were made neutropenic by IP injection of 150 mg/kg cyclophosphamide into the left abdomen. Mice were inoculated with 10⁵ CFU of E. coli strain MG1655 by intramuscular injection into the thigh 30 minutes prior to phage treatment. Each individual crPhage or cocktail of 3 crPhages were administered by intramuscular injection into the same thigh with 100 μL of crPhage solution, corresponding to a dose of 2.0×10¹¹ PFU/dose of crT7, 4.0×10¹¹ PFU/dose of crT7M, 2.0×10¹⁰ PFU/dose of crT4, or the cocktail containing 1.0×10¹⁰ PFU/dose of each phage. After injection with each crPhage, CFU reductions measured approximately 2-log for crT4 (FIG. 17B), 3-log for crT7M (FIG. 17C) and >5-log for both crT7 (FIG. 17A) and the combined crPhage cocktail (FIG. 17D). Taken together these data exemplify that crPhages have the potential to be highly effective antimicrobial agents in vivo.

### Example 13. Persistence and Distribution of crPhage in the Urinary and GI Tract

The objectives of in vivo studies were to evaluate the persistence and distribution of engineered bacteriophages (crPhages) over time in both target tissues and distal organs (including the bladder, kidney, blood, liver, spleen, and gastrointestinal [GI] tract) after intraurethral (IU) or oral administration in healthy murine models by titration and quantitative polymerase chain reaction (qPCR), respectively.

A cocktail of clustered regularly spaced short palindromic repeats (CRISPR)-enhanced bacteriophages crT7 and crT7M was used in Study 16(a) and a cocktail of CRISPR-enhanced bacteriophages crT7, crT7M, and crT4 was used in Study 16(b).

The 3 CRISPR-enhanced bacteriophages were constructed to carry a similar deoxyribonucleic acid (DNA) sequence encoding functional self-targeting CRISPR ribonucleic acid (RNA) embedded in the wild-type phage genome. crT4 was engineered by deleting the hoc gene and replacing it with a CRISPR ribonucleic acid (crRNA) cassette. crT7 was engineered by deleting gp0.7, gp4.3, gp4.5 and gp4.7 and replacing it with a crRNA cassette. crT7M was engineered by deleting gp0.6, 0.65, 0.7, gp4.3 and gp4.5 and replacing it with a crRNA cassette. Each phage was engineered with a similar crRNA cassette that contained two elements: (1) a leuO transcription factor gene derived from E. coli downstream of a synthetic promoter, and (2) a repeat-spacer-repeat encoding a crRNA targeting the ftsA gene downstream of a synthetic promoter.

In Study 16(a), female CD-1 mice were treated with approximately 1.0×10⁹ plaque forming units (PFU)/dose/phage of a crT7/crT7M cocktail suspended in ix Tris-buffered saline (pH 7.4) by IU instillation. The IU instillation was done by placement of a silicone-tipped syringe into the urethra and solution was injected directly into the bladder. Each mouse was dosed with an approximately 50 μL of vehicle or phage cocktail by IU instillation while under isoflurane anesthesia. At time points of 0 (immediately following IU administration), 0.5, 1, 6, 12, 24 and 72 hours post-inoculation, 3 mice per time point were sacrificed and the bladder, kidney, blood, liver and spleen were collected and phage content determined. crPhages were quantified as a pooled measurement using conventional phage titration against a known host that is susceptible to both crT7 and crT7M.

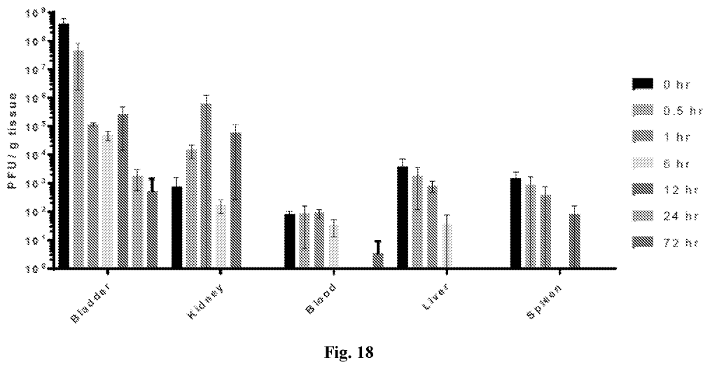

Phage titration detection of crPhages following IU administration showed that active crPhages were detected up to 72 hours after dosing, with no increase in phage levels during this time period, which was expected due to the lack of a target E. coli replication host in the normal tissues and the inability to replicate in mammalian cells. The crPhage levels decreased over time in bladder and were undetectable in kidney, liver, blood, and spleen 72 hours post IU instillation (Table 22 and FIG. 18).

TABLE 22
Study 16(a): Phage titration detection of crPhages in the murine urinary
tract and other organs after single IU dose

| Time | | Bladder (PFU/g tissue) | Kidney (PFU/g tissue) | Blood (PFU/mL) | Liver (PFU/g tissue) | Spleen (PFU/g tissue) |
|---|---|---|---|---|---|---|
| point | | Mean (SEM) | Mean (SEM) | Mean (SEM) | Mean (SEM) | Mean (SEM) |
| 0 | hr | 4.0 × 10 [symbol] (2.0 × 10 [symbol] ) | 7 × 10 [symbol] (7 × 10 [symbol] ) | 8 × 10 [symbol] (2 × 10 [symbol] ) | 2 × 10 [symbol] (2 × 10 [symbol] ) | 1 × 10 [symbol] (9 × 10 [symbol] ) |
| 0.5 | hr | 4.4 × 10⁷ (4.2 × 10 [symbol] ) | 1 × 10 [symbol] (6 × 10 [symbol] ) | 8 × 10 [symbol] (8 × 10 [symbol] ) | 1 × 10 [symbol] (1 × 10 [symbol] ) | 8 × 10 [symbol] (8 × 10 [symbol] ) |
| 1 | hr | 1.16 × 10⁷ (1.61 × 10 [symbol] ) | 5 × 10 [symbol] (5 × 10 [symbol] ) | 9 × 10 [symbol] (3 × 10 [symbol] ) | 5.0 × 10 [symbol] (2.0 × 10 [symbol] ) | 4 × 10 [symbol] (4 × 10 [symbol] ) |
| 6 | hr | 4.8 × 10⁴ (1.7 × 10 [symbol] ) | 1 × 10 [symbol] (8 × 10 [symbol] ) | 3 × 10 [symbol] (2 × 10 [symbol] ) | 2 × 10 [symbol] (2 × 10 [symbol] ) | BLD |
| 12 | hr | 3 × 10 [symbol] (2 × 10 [symbol] ) | 5 × 10 [symbol] (5 × 10 [symbol] ) | BLD | BLD | 8 × 10 [symbol] (8 × 10 [symbol] ) |
| 24 | hr | 2 × 10 [symbol] (1 × 10 [symbol] ) | BLD | BLD | BLD | BLD |
| 72 | hr | 5 × 10 [symbol] (5 × 10 [symbol] ) | BLD | 3 × 10 [symbol] (3 × 10 [symbol] ) | BLD | BLD |

BLD = below the limit of detection;
IU = intraurethral;
PFU = plaque-forming unit;
SEM = standard error of the mean [symbol]
indicates data missing or illegible when filed

Because phage titration is difficult to use in the quantification of individual crPhages present in a given sample due to their overlapping host ranges, a qPCR-based method was developed and validated for the detection of each crPhage within a given cocktail. Linear dilutions of known quantities (ng or copies) of crPhage DNA were mixed into background mouse genomic DNA (gDNA) and the curve was used to define the quantity of phage copies/per total DNA in the test samples. The limit of detection was set by the highest quantitation cycle (Cq) detected in the linear range of the standard curve.

Quantitative PCR is a highly specific method to detect and quantify DNA, enabling detection levels down to 50 copies/ng of total DNA in complex samples, and is theoretically able to measure the total amount of each engineered crPhage within samples as the primers are designed to recognize a specific phage genome containing an identical crRNA cassette insert. Although it is a highly specific and sensitive method of detection, qPCR does not distinguish active versus non-active phage, whereas plaquing assays are able to do so. As proof-of-principle, Study 16(b) was conducted.

In Study 16(b), mice were gavaged with 0.2 mL of 6% sodium bicarbonate to reduce stomach acid levels approximately 30 minutes prior to crPhage dosing to mitigate potential phage degradation. A single dose of 2.7×10⁹ PFU total of each crT7, crT7M and crT4 in 200 μL 1× Tris-buffered saline (pH 7.4) was administered by oral gavage to N=3 mice per condition/time point. At time points of pre-dose, 1, 2, 4, 8, 12, 24, 48 and 72 hours post-inoculation, 3 mice per time point were sacrificed and phage content was determined in the duodenum, jejunum, ileum, cecum, colon, kidney, blood, liver and spleen.

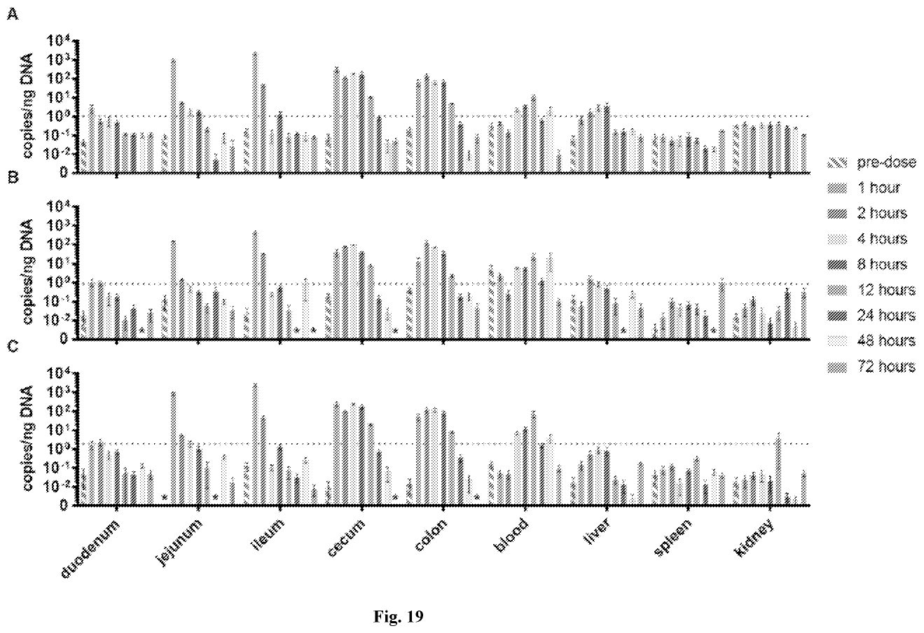

It was possible to successfully detect each crPhage by qPCR during transit through the GI tract across 48 hours. Quantitative PCR detection of crPhages following oral gavage showed that by 72 hours, crPhage is successfully cleared and was not quantifiable over background in any of the tissues examined (Table 23 and FIG. 19). These data corroborate the observation of loss of crPhages over time after a single IU administration as quantified by titration. Notably, these data suggest that oral administration of crPhages results in systemic exposure, specifically in blood and liver tissues, as observed following IU administration.

TABLE 23
Study 16(b): Quantitative PCR detection of crPhages in the murine GI tract and
other organs after single oral dose (copies/ng DNA)

| | | | | | | | | | |
|---|---|---|---|---|---|---|---|---|---|
| | Duodrnum | Jejunum | Deum | Cocum | Colon | Bleed | Liver | Spleen | Kidney |
| Time | Mean | Mean | Mean | Mean | Mean | Mean | Mean | Mean | Mean |
| point | (SEM) | (SEM) | (SEM) | (SEM) | (SEM) | (SEM) | (SEM) | (SEM) | (SEM) |

crT7

| | | | | | | | | | |
|---|---|---|---|---|---|---|---|---|---|
| Pre-dose | 0.044 (0.17) | 0.083 (0.020) | 0.18 (0.045) | 0.086 (0.033) | 0.21 (0.062) | 0.31 (0.33) | 0.068 (0.034) | 0.083 (0.034) | 0.31 (0.029) |
| 1 hr | 2.8 (1.3) | 1000 (180) | 2200 (350) | 110 (93) | 66 (26) | 0.42 (0.087) | 0.73 (0.22) | 0.078 (0.031) | 0.41 (0.081) |
| 2 hr | 0.56 (0.18) | 5.4 (0.52) | 44 (62) | 120 (17) | 140 (42) | 0.14 (0.059) | 1.75 (0.72) | 0.054 (0.022) | 0.26 (0.038) |
| 4 hr | 0.56 (0.15) | 2.1 (0.69) | 6.4 (5.5) | 150 (22) | 70 (14) | 1.7 (0.56) | 2.6 (0.89) | 0.053 (0.023) | 0.30 (0.084) |
| 8 hr | 0.49 (0.15) | 1.7 (9.47) | 1.9 (0.40) | 180 (57) | 65 (22) | 3.3 (0.78) | 3.5 (1.7) | 0.089 (0.060) | 0.37 (0.13) |
| 12 hr | 0.12 (0.014) | 0.22 (0.051) | 0.83 (0.40) | 11 (1.1) | 4.7 (0.42) | 11 (3.8) | 0.16 (0.048) | 0.066 (0.016) | 0.42 (0.088) |
| 24 hr | 0.11 (9.018) | 0.0053 | 0.13 (0.022) | 0.76 (0.16) | 0.41 (0.11) | 0.61 (0.17) | 0.17 (0.064) | 0.021 | 0.27 (0.035) |
| | | (0.0043) | | | | | | (0.0068) | |
| 48 hr | 0.10 (0.028) | 0.082 (0.044) | 0.10 (0.044) | 0.035 (0.022) | 0.010 | 2.1 (0.05) | 0.17 (0.053) | 0.020 | 0.25 (0.056) |
| | | | | | (0.0051) | | | (0.0067) | |
| 72 hr | 0.11 (0.029) | 0.027 (0.026) | 0.077 (9.014) | 0.052 (0.018) | 0.076 (0.035) | 0.0068 | 0.078 (0.030) | 0.18 (0.018) | 0.11 (0.011) |
| | | | | | | 0.0078 | | | |

crT4

| | | | | | | | | | |
|---|---|---|---|---|---|---|---|---|---|
| Pre-dose | 0.018 (0.012) | 0.13 (0.088) | 0.025 (0.016) | 0.21 (0.055) | 0.40 (0.12) | 5.9 (2.2) | 0.12 (0.083) | 0.0037 | 0.014 |
| | | | | | | | | (0.0027) | (0.0067) |
| 1 hr | 1.0 (0.4) | 140 (12) | 440 (80) | 30 (16) | 14 (6.4) | 2.1 (0.82) | 0.082 (0.051) | 0.014 (0.011) | 0.044 (0.028) |
| 2 hr | 0.85 (0.29) | 1.5 (0.083) | 34 (2.2) | 79 (3.6) | 180 (3.5) | 0.26 (0.14) | 1.4 (0.40) | 0.098 (0.049) | 0.12 (0.060) |
| 4 hr | 0.28 (0.12) | 0.56 (0.18) | 4.7 (4.2) | 84 (12) | 78 (8.6) | 4.4 (8.6) | 0.91 (0.20) | 0.051 (0.021) | 0.039 (0.050) |
| 8 hr | 0.18 (0.068) | 0.31 (0.067) | 0.51 (0.18) | 37 (6.5) | 36 (9.6) | 5.2 (1.3) | 0.48 (0.20) | 0.067 (0.028) | 0.0072 |
| | | | | | | | | | (0.0061) |
| 12 hr | 0.0087 | 0.053 (0.026) | 0.031 (0.030) | 6.2 (2.1) | 2.3 (0.34) | 24 (8.8) | 0.084 (0.065) | 0.045 (0.025) | 0.082 (0.021) |
| | (0.0048) | | | | | | | | |
| 24 hr | 0.041 (0.023) | 0.36 (0.18) | 0.0010 (0.0) | 0.15 (0.083) | 0.18 (0.062) | 1.3 (0.45) | 0.0010 (0.0) | 0.010 (0.014) | 0.31 (0.18) |
| 48 hr | 0.010 (0.0) | 0.008 (0.27) | 0.81 (0.70) | 0.026 (0.015) | 0.10 (0.078) | 20 (16) | 0.25 (0.096) | 0.0010 (0.0) | 0.0044 |
| | | | | | | | | | (0.0034) |
| 72 hr | 0.023 (0.015) | 0.034 (0.022) | 0.010 (0.0) | 0.010 (0.0) | 0.041 (0.040) | 0.98 (0.038) | 0.046 (0.031) | 1.0 (0.64) | 0.33 (0.17) |

crT7M

| | | | | | | | | | |
|---|---|---|---|---|---|---|---|---|---|
| Pre-dose | 0.040 (0.042) | 0.0010 (0.0) | 0.13 (0.063) | 0.010 | 0.015 (0.011) | 0.18 (0.015) | 0.016 (0.015) | 0.042 (0.027) | 0.018 (0.013) |
| | | | | (0.0000) | | | | | |
| 1 hr | 1.7 (0.78) | 880 (140) | 2400 (380) | 240 (67) | 52 (20) | 0.051 (0.020) | 0.15 (0.075) | 0.084 (0.036) | 0.024 (0.015) |
| 2 hr | 2.4 (0.94) | 5.7 (0.49) | 47 (0.8) | 100 (7.4) | 120 (33) | 0.048 (0.023) | 0.54 (0.23) | 0.12 (0.041) | 0.063 (0.016) |
| 4 hr | 0.81 (0.29) | 2.2 (0.72) | 7.2 (5.8) | 180 (33) | 110 (22) | 5.3 (1.4) | 0.81 (0.28) | 0.031 (0.015) | 0.042 (0.020) |
| 8 hr | 0.72 (0.20) | 1.1 (0.38) | 1.3 (0.40) | 180 (44) | 82 (22) | 11 (3.1) | 0.81 (0.37) | 0.072 (0.022) | 0.021 (0.018) |
| 12 hr | 0.040 (0.049) | 0.11 (0.068) | 0.070 (0.044) | 20 (2.1) | 8.3 (0.94) | 74 (29) | 0.026 (0.011) | 0.31 (0.074) | 3.6 (3.5) |
| 24 hr | 0048 (0.017) | 0.0010 (0.0) | 0.032 (0.016) | 0.70 (0.22) | 0.36 (0.18) | 1.6 (0.52) | 0.014 | 0.014 | 0.0032 |
| | | | | | | | (0.0092) | (0.0092) | (0.0015) |
| 48 hr | 0.14 (0.036) | 0.41 (0.11) | 0.27 (0.079) | 0.065 (0.047) | 0.022 (0.017) | 3.0 (1.8) | 0.0024 | 0.065 (0.020) | 0.020 |
| | | | | | | | (0.0014) | | (0.0010) |
| 72 hr | 0.048 (0.023) | 0.016 (0.015) | 0.0078 | 0.061 (0.0) | 0.001 (0.0) | 0.1 (0.035) | 0.17 (0.030) | 0.041 (0.013) | 0.054 (0.019) |

DNA= deoxyribonucleic acid;
GI = gastrointestinal;
PCR - polymerization chain reaction;
SEM = standard error of the mean

Detection of crPhages using titration and qPCR methods showed crPhage is successfully cleared 72 hours post IU and oral administration, respectively. Both studies corroborated the loss of crPhages over time as well as the systemic exposure achieved, specifically in blood and liver tissues, following the administration of crPhages in mice.

### Example 14. A 7-Day Repeat Dose Toxicity Study in Mice

The objective of this study was to evaluate the toxicity in female CD-1 mice following intravenous (IV) or intraurethral (IU) administration of crT37 Phage for 7 consecutive days.

The test article in this study was a cocktail containing a mixture of 2 different crPhages: crT7 and crT7M (this mixture is referred to as crT37 Phage).

The experimental crT37 Phage was administered either IV or IU (0.5×10¹¹ PFU/dose/phage) once daily for 7 consecutive days to female Crl:CD-1 mice. Groups 1 and 2 (9 female mice/group) were dosed with 0.1 mL of vehicle (1×TBS) or test article in a tail vein. Groups 3 and 4 (9 female mice/group) were dosed with 0.05 mL of vehicle or test article into the bladder using a catheter.

TABLE 24A
Dosing regimen

| | | | | | Number of |
|---|---|---|---|---|---|
| | | | | | Animals |
| | | | | Dose | for |
| Dose | Number of | Test | | Volume | Necropsy |
| Group | Animals | Article | Dose Route | (mL/animal) | Day 8 |
| 1 | 9 | Vehicle | Intravenous | 0.1 | 6 |
| 2 | 9 | crT37 Phage | Intravenous | 0.1 | 6 |
| 3 | 9 | Vehicle | Intraurethral | 0.05 | 6 |
| 4 | 9 | crT37 Phage | Intraurethral | 0.05 | 6 |

Animals were monitored for clinical signs twice daily over the duration of the study. Detailed clinical observations were performed once during the predose period and prior to necropsy on Day 8. Body weights were measured once during the predose period and on Days 1, 3, and 7. Food consumption was measured during the 7-day dosing period. On Day 8, six animals per group were randomly chosen for necropsy, and the remaining three were discarded without necropsy. At necropsy, body weights were collected from animals fasted for at least 4 hours and were used for calculation of organ weights relative to body weight. Clinical pathology assessments, hematology (3 mice/group) and serum chemistry parameters (3 mice/group), were performed on the day of the scheduled necropsy. Postmortem assessment included necropsy and measurement of selected organ weights. A full tissue list was collected at necropsy. Collected tissues were sectioned with one section frozen in liquid nitrogen (stored frozen at ≤−70° C.) and a second section was preserved in 10% neutral-buffered formalin.

There was no crT37 Phage-related mortality or moribundity, and no crT37 Phage-related effects on body weight. Animals were normal at all observations.

Phage-related effects on hematology were limited to a lower hemoglobin level and decreases in other RBC mass-related parameters, increased reticulocyte counts, and decreased eosinophil count in the Phage IV-treated group. Phage-related effects on serum chemistry were limited to higher cholesterol and triglyceride levels in the Phage IU-treated group (Table 24).

TABLE 24
Mean Changes in Blood and Organ Parameters at Day 8
Relative to Start Date: crPhage-Related Effects

| | Mean (SD) change at Day 8 relative to start date | |
|---|---|---|
| Parameter | Vehicle | crPhage |

Clinical chemistry (n = 3 animals/group)

| | | |
|---|---|---|
| Cholesterol (mg/dL) | 124.0 (17.6) ^{a} | 165.0 (6.9) ^{b} |
| Triglycerides (mg/dL) | 127.3 (6.4) ^{a} | 206.3 (38.0) ^{b} |

CRISPR = clustered regularly interspaced short palindromic repeats;
crPhage = CRISPR-enhanced phage;
SD = standard deviation
^{a} Significant (p <0.05) per an analysis off variance test on all groups.
^{b} Significant (p <0.05) per a Dunnet 2-sided test.

Phage-related effects on organ weights (absolute and relative to body and brain weights) consisted of increased spleen and kidney weights and decreased lung weights in the Phage IV-treated group.

Once daily IV and IU administration of crT37 Phage was well tolerated. Possible test article-related effects in the Phage IV-treated group were limited to decreases red blood cell mass-related parameters, increased reticulocyte counts, decreased eosinophils, and increased spleen, kidney, and decreased lung weights. Possible test article-related effects in the Phage IU-treated group were limited to higher cholesterol and triglyceride levels.

### Example 15. In Vitro Kill Curves

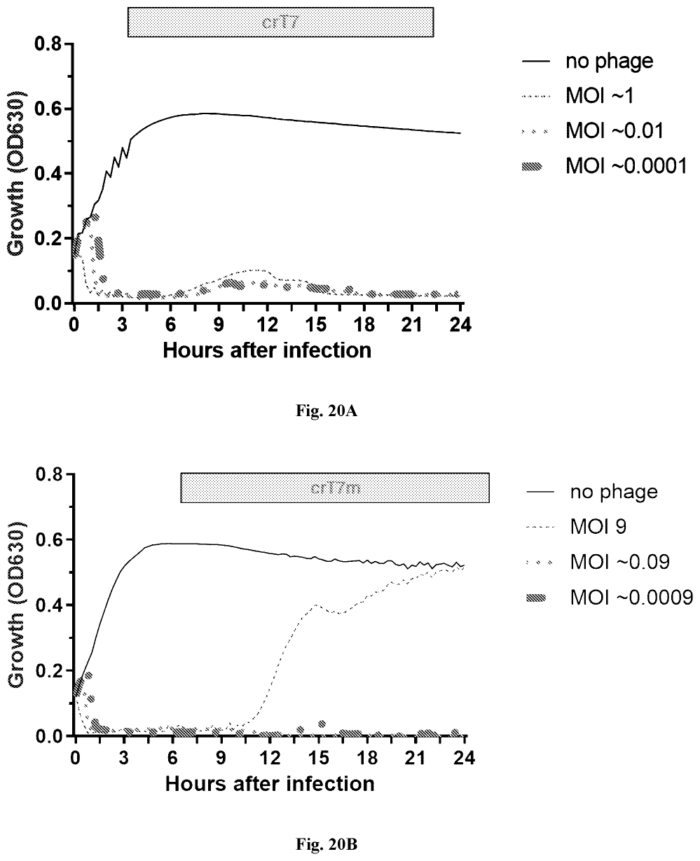

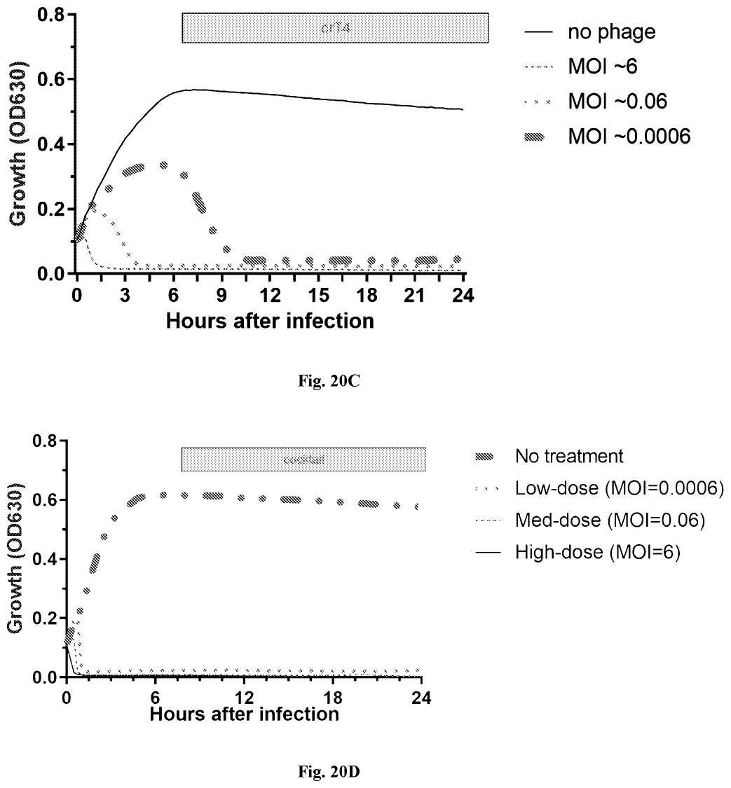

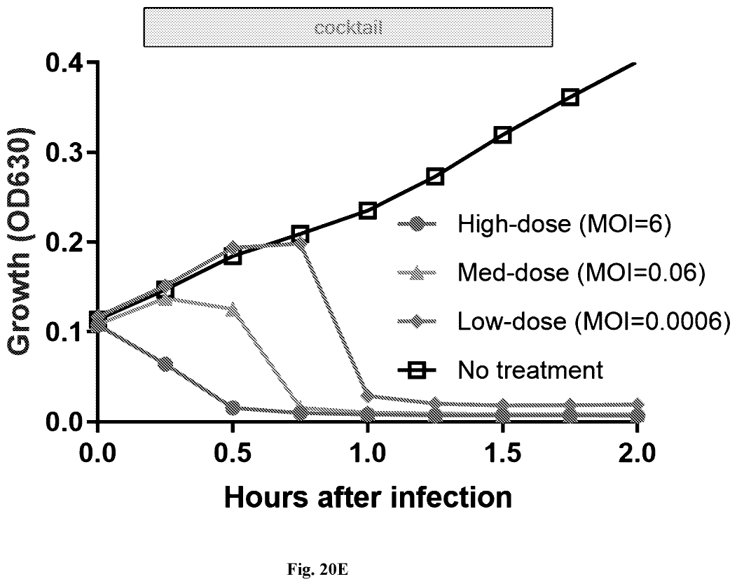

Each crPhage was produced by standard lytic amplification, filtration and left suspended in the original growth media (Lysogeny broth [LB] broth). All experiments were conducted in LB broth. E. coli strain MG1655 was grown to mid-log phase and then mixed to the indicated MOI with either crPhage, crPhage cocktail or LB only negative control. Treated populations were grown under aerobic, shaking conditions at 37° C. for 24 hours in a plate reader to monitor growth of treated populations by optical density ([OD] 630 nm) (FIG. 20A-FIG. 20E). All crPhages lysed the target strain independent of MOI. An emergent resistant population in the crT7M high-dose population (FIG. 20B) and in the crT4 low-dose population (MOI=0.0006, FIG. 3C) was observed by 24 hours of continuous culture in the presence of the initial phage dose. However, when challenged with a cocktail of crT7, crT7M and crT4, no resistant population was observed (FIG. 20D).

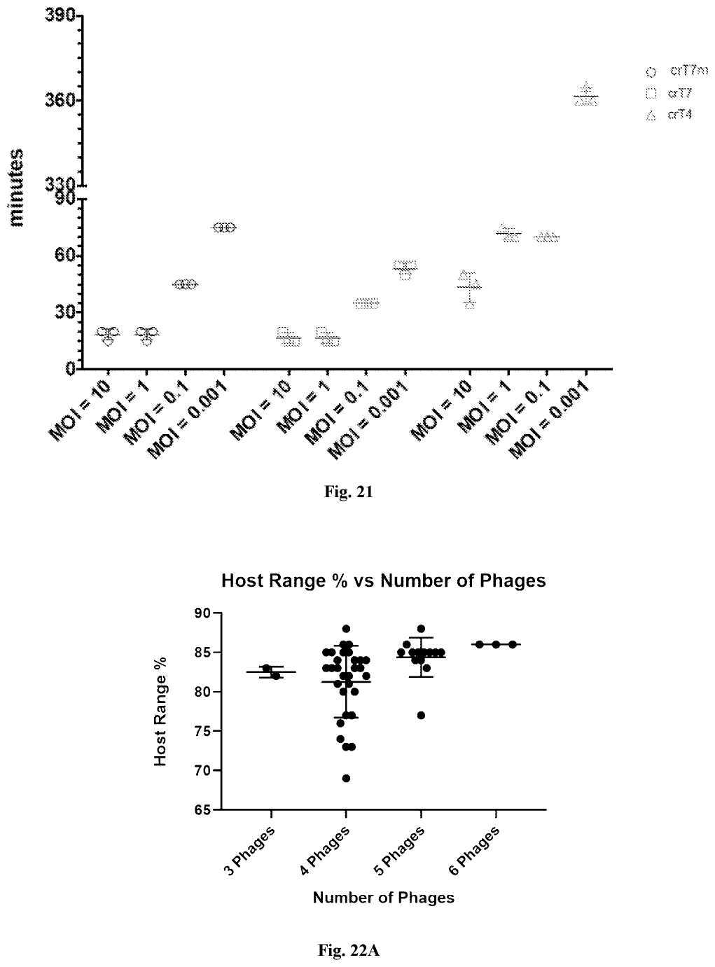

A dose-dependent relationship was observed between concentrations of crPhage and observable time-to-lysis. A follow-on experiment was conducted to quantify time-to-lysis compared to MOI (FIG. 21). Time-to-lysis is the time at which the first derivative of the growth curve reaches 0 after the bacterial population crashes due to presumed lytic phage amplification. For all 3 crPhages tested, MOI in excess of 1.0 appeared to result in fastest time-to-lysis, presumably being limited by the lytic period of each phage. The observed time-to-lysis of approximately 15-20 minutes for crT7M and crT7 and 45-50 minutes for crT4 largely agree with the values known for the wild-type lytic phages T7 (˜17 minutes; Nguyen and Kang, 2014), T7M (˜15-20 minutes; similar to T7) and T4 (35 minutes; (Dressman and Drake, 1999), respectively.

### Example 16: Different Phage Cocktails and the Effect on Host Range

Ten crPhage candidates identified in Table 25 were assigned into different cocktail combinations of 3 to 6 phages. Each combination was tested against a panel of E. coli isolates by mixing bacteria and phage at a defined MOI and measuring optical density growth curves for approximately 18 hours. Phages were added at an MOI of 0.1 to 10 with material prepared from purified phage preparations. After approximately 18 hours, the total area under the curve (AUC) was calculated and compared to no phage added controls. AUC ratios under 0.65 of phage to no phage added controls were designated as a positive infection event. The total number of positive infection events was divided by the total number of strains tested to determine host range percentage. The cocktail tested with a combination of 3 or more phages showed a host range of greater than 80%, as depicted in FIG. 21.

TABLE 25
Phage used

| Phage | ATCC Accession Number/Patent Deposit Number | Host range |
|---|---|---|
| P00ex | PTA-126324 | 47% |
| P00dd | PTA-126322 | 48% |
| P004k | PTA-126319 | 41% |
| P0031 | PTA-126315 | 27% |
| P0071 | PTA-126320 | 54% |
| P00jc | PTA-126325 | 40% |
| P0078 | PTA-126321 | 25% |
| P0045 | PTA-126318 | 19% |
| p0033L | PTA-126316 | 51% |
| P00e8 | PTA-126323 | 47% |

### Example 17: LBP-EC01 Cocktail is Effective Across a Wide Host Range

crPhage cocktail LBP-EC01 is composed of phages identified in Table 26 was tested against a panel of 176 E. coli isolates by mixing bacteria and phage at a defined MOI and measuring optical density growth curves for approximately 20 hours. Phages were added at an MOI of 0.0000001 to 10 with material prepared from purified phage preparations. After approximately 20 hours, the total area under the curve (AUC) was calculated and compared to no phage added controls. AUC ratios under 0.65 of phage to no phage added controls were designated as a positive infection event. The total number of positive infection events was divided by the total number of strains tested to determine host range percentage.

| Phage | ATCC Accession Number/Patent Deposit Number | Host range |
|---|---|---|
| P00ex | PTA-126324 | 47% |
| P004k | PTA-126319 | 41% |
| P0031 | PTA-126315 | 27% |

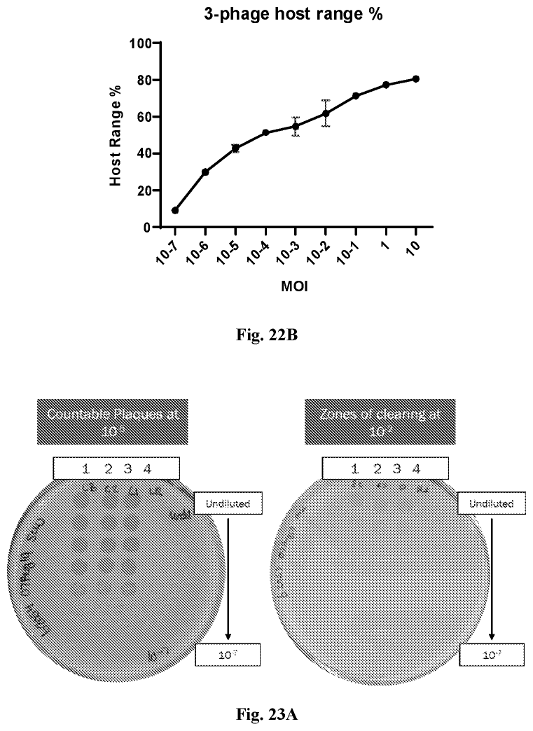

The results are depicted in FIG. 22. At an MOI of 10⁻⁴ or greater, the LBP-EC01 had a host range of greater than 50%.

LBP-EC01 was tested in technical triplicate against a panel of E. coli isolates by preparing a double agar overlay of each target strain and spotting 10-fold serial dilutions of the LBP-EC01 on the agar overlay. After incubation overnight, lysis by the spotted LBP-EC01 preparation was assessed by observation of plaques or zones of clearing. Zones of clearing were considered as serial dilutions where lysis was observed without formation of plaques. The lowest dilution at which zones of clearing lysis was observed was denoted as ‘ZC[dilution number]’. EC01 technical triplicates are in lanes 1, 2, and 3 and diluent, Lactated Ringer's, in lane 4 on the plate.

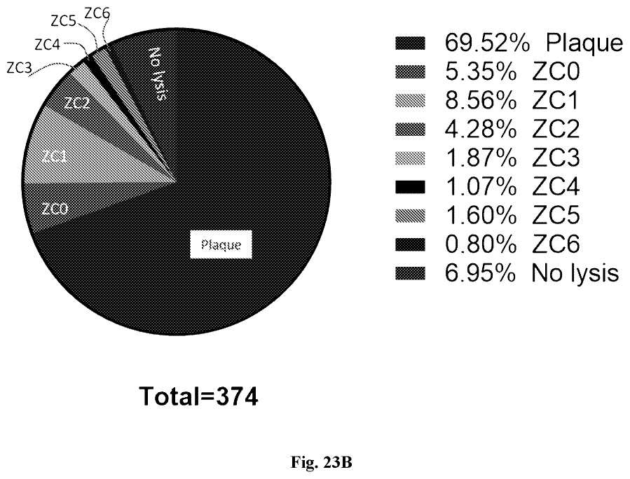

The results of this assay are depicted in FIG. 23A and quantified in FIG. 23B. At a MOI of 10⁻⁵, countable plagues were observed. At an MOI of 10⁻², zone of clearing were observed in the agar overlay. LBP-EC01 contains PTA-126324, PTA-126319, and PTA-126315, and the experiment was carried out with a 1e9 PFU/mL input titer.

### Example 18. A Multi-Center Randomized, Double-Blind Study to Assess the Safety, Tolerability, Pharmacokinetics and Pharmacodynamics of Phage Cocktail in Patients with Lower Urinary Tract Colonization Caused by E. coli

### Proposed Indication

A Phase 1b, multicenter, randomized, double-blind study to assess the safety, tolerability, pharmacokinetics (PK), and pharmacodynamics (PD) of a LBP-EC01 in patients with lower urinary tract colonization caused by E. coli. The study is conducted in approximately 6 sites in the US. LBP-EC01 is a biologic compound comprising a cocktail of up to 6 distinct CRISPR-enhanced obligate lytic bacteriophages (e.g. p00ex-2 (ATCC number PTA-126324), p0031-8 (ATCC number PTA-126315), p004k-5 (ATCC number PTA-126319)) that selectively lyse E. coli, including MDR, ESBL, and carbapenem-resistant, as well as non-MDR and non-carbapenem-resistant, strains. The mechanism is 2-fold: 1) obligate lytic bacteriophages kill target cells via lytic replication and 2) CRISPR-Cas3 kills by targeting and destroying chromosomal DNA to prevent cellular replication.

### Study Design

This is a first-in-human study in patients with urinary tract colonization by E. coli who have indwelling urinary catheters, or who require intermittent urinary catheterization, and/or patients with asymptomatic bacteriuria caused by E. coli.

No formal hypothesis is tested in this Phase 1b study. Approximately 30 patients are randomized 2:1 to receive either the maximum feasible dose twice daily (BID) of the LBP-EC01 or inert placebo for 7 days by intravesical catheter.

The study will consist of a Screening Period of up to 21 days. On Day −1 patients will either be hospitalized or in a facility that can provide proper administration of study drug and then randomized to LBP-EC01 or placebo. The entire 7-day Treatment Period in which patients will receive 13 doses of Investigational Medicinal Product (IMP) will be conducted with the patient in the clinic/hospital. In circumstances where the patient may not be able to be in clinic/hospital for the full 7 days of treatment, upon consultation between the treating physician and the study Medical Monitor, patients may be considered to be treated and monitored in clinic/hospital for Days 1-3 and then may be allowed to come back to the clinic/hospital for the PM dose on Day 3 and the remaining treatment from Days 4-7. The End of Treatment (EOT) will be after the 13th dose on Day 7, after which the patient may be released from the clinic/hospital. The patient will return on Day 14 (±3 days) for the Day 14 Visit, and on Day 28 (±3 days) for the Day 28 Visit. Adverse Event (AE) data will be collected throughout the study, including Day 28 through Day 35. At Day 35, the patient will be contacted by telephone to assess any AEs or lab abnormalities since the Day 28 Visit. At the discretion of the investigator, the patient may be asked to return to the clinic/hospital for a Day 35 Visit (±3 days). Day 35 is the End of Study (EOS). Sensitivity of E. coli from patient's urine samples to crPhage cocktail is tested to identify persisting or recurrent strains during the conduct of the study. There are no planned changes to the crPhage cocktail while a patient is being treated or in follow-up. This data is compiled at the completion of the study to determine if any potential escape or resistance to LBP EC01 has developed to help inform possible changes to LBP-EC01 design in future clinical testing.

### Dose

Intra-bladder administration twice daily dosing (BID) with the maximal feasible dose of LBP-EC01 defined by the manufacturing process.

### Study Population

Male or female patients ≥18 years of age with a documented UTI caused by E. coli within the past 12 months and a current lower urinary tract colonization (≥10³ CFU/mL) caused by E. coli and who meet at least one of the following:
Has an indwelling urinary catheter
Requires intermittent catheterization
Has medical documentation of a history of asymptomatic bacteriuria (i.e., lower urinary tract colonization) with E. coli at least once in the past 12 months

Patients have experience with catheterization or have Medical Monitor approval if the patient has limited, or no prior experience with catheterization. Patients are in good general health as evidenced by medical history and physical examination. Women of childbearing potential and men with female partners of childbearing potential use two forms of effective contraception, at least 1 of which is a physical barrier method, during the study and continued for 2 weeks after completing the study.

Patients with clinical signs or symptoms of an active UTI or other infection requiring antimicrobial treatment or who have received Gram-negative bacteria antimicrobials within the past 14 days are not allowed to enroll. In addition, patients with a surgically-modified bladder, except for a repaired ruptured bladder, who have severe autonomic dysreflexia, or who have active severe, progressive or uncontrolled renal, hepatic, hematologic, gastrointestinal, endocrine, pulmonary, cardiac, or neurologic disease are also excluded from the study.

### Study Objectives and Endpoints

The primary objective of Study is to evaluate the safety, tolerability and PK of LBP-EC01 in patients ≥18 years of age with lower urinary tract colonization caused by E. coli.

The secondary objective is to evaluate the pharmacodynamics (PD) of crPhage cocktail.

Exploratory objective is to explore the influence of crPhage cocktail on the urinary tract microbiota.

Primary endpoints for the study include:
Safety and tolerability analysis of AEs
PK analysis

Secondary endpoints for the study include:
Reduction in urinary E. coli burden at EOT (Day 7), Day 14, and EOS (Day 28)
Time to 1 log reduction in urinary E. coli count from baseline
Recurrence of E. coli colonization or incidence of infection based on clinical signs and symptoms
Evaluation of possible immunogenicity by measuring changes in immunoglobulin A (IgA), immunoglobulin E (IgE), immunoglobulin G (IgG), and immunoglobulin M (IgM) levels

### Study Duration

Estimated duration of enrollment, treatment period and follow-up is approximately 5 months.

### Patient Duration

Study duration for patients will be up to 56 days, which includes up to 21 days for screening, 7 days of Investigational Medicinal Product (IMP) treatment, a Day 14 assessment, a Day 28 assessment (28 days after first dose), and at EOS (35 days after first dose). Patients will be in the clinic or hospital the evening prior to receiving the first dose of treatment and throughout the 7 days of treatment.

Once the study is complete, patients should resume their regular medications and any catheters installed specifically for the conduct of the study are replaced by new catheters at the discretion of their physician.

### Anticipated Risks to Human Subjects

There are no known risks of LBP-EC01, as it is not evaluated in clinical studies. However, the following are identified as potential risks based on phage clinical literature: immunologic reactions, increase in endotoxin exposure, and transfer of bacterial virulence factors or antibiotic resistance genes. To date, clinical experience with wild-type phage therapies has not revealed any signals.

Risks in this study are perceived to be low due to known lack of toxicity of phage therapy, whereas the benefits of establishing dosing and PK parameters as well as safety, are considered essential to progressing this type of therapy to the clinic. A potential concern for safety is the possibility for endotoxin release associated with active bacterial lysis. While LBP-EC01 contains small amounts of endotoxin, the manufacturing specifications provide strict limits to the amount of endotoxin in the final drug product. The endotoxin exposure is similar to that seen with rapidly effective bactericidal antibiotic therapy. The clinical study includes close monitoring of the effects of endotoxin release.

Instrumentation or instillation of solutions into the bladder could induce autonomic dysreflexia in certain spinal injuries. Patients with severe autonomic dysreflexia will be excluded. Any instrumentation will be minimized and the intravesical dose volume will be approximately 60 mL and warmed to room temperature before instillation. All procedures related to drug preparation, instrumentation and catheter handling will be performed under sterile conditions to avoid introduction of infection. A potential safety concern with bacteriophage therapy (both wild type and CRISPR-enhanced bacteriophage), is the potential to transfer bacterial virulence factors or antibiotic resistance genes. The bacteriophages selected for LBP-EC01 production were screened to exclude those with virulence genes or antibiotic resistance genes.

While preferred embodiments of the present disclosures have been shown and described herein, it will be obvious to those skilled in the art that such embodiments are provided by way of example only. Numerous variations, changes, and substitutions will now occur to those skilled in the art without departing from the disclosures. It should be understood that various alternatives to the embodiments of the disclosures described herein may be employed in practicing the disclosures. It is intended that the following claims define the scope of the disclosures and that methods and structures within the scope of these claims and their equivalents be covered thereby.

What is claimed is:

1. A nucleic acid sequence comprising (a) a first CRISPR array designed to be operable with a first Type I CRISPR-Cas system, and (b) a second CRISPR array designed to be operable with a second Type I CRISPR-Cas system, wherein the first Type I CRISPR-Cas system and the second Type I CRISPR-Cas system are different Type I CRISPR-Cas systems.

2. The nucleic acid sequence of claim 1, wherein the first CRISPR array comprises a first spacer sequence and the second CRISPR array comprises a second spacer sequence.

3. The nucleic acid sequence of any one of claims 1-2, wherein the first CRISPR array and the second CRISPR array further comprises at least one repeat sequence.

4. The nucleic acid sequence of claim 3, wherein the at least one repeat sequence is operably linked to the first spacer sequence at either its 5′ end or its 3′ end and/or the second spacer sequence at either its 5′ end or its 3′ end.

5. The nucleic acid sequence of any one of claims 2-4, wherein the first and/or second spacer sequence is complementary to a target nucleotide sequence of an essential gene in a target bacterium.

6. The nucleic acid sequence of claim 5, wherein the target nucleotide sequence comprises all or a part of a promoter sequence of the essential gene.

7. The nucleic acid sequence of claim 5, wherein the target nucleotide sequence comprises all or a part of a nucleotide sequence located on a coding strand of a transcribed region of the essential gene.

8. The nucleic acid sequence of any one of claims 5-7, wherein the essential gene is ftsA.

9. The nucleic acid sequence of any one of claims 2-4, wherein the first and/or second spacer sequence is complementary to a target nucleotide sequence in a non-essential gene.

10. The nucleic acid sequence of any one of claims 2-4, wherein the first and/or second spacer is completely to a target nucleic acid sequence in a noncoding sequence.

11. The nucleic acid sequence of any one of claims 1-10, wherein the first CRISPR array and the second CRISPR array are on same nucleic acid sequence.

12. The nucleic acid sequence of any one of claims 1-11, wherein the nucleic acid sequence further comprises a leuO coding sequence.

13. The nucleic acid sequence of any one of claims 1-12, wherein the nucleic acid sequence further comprises a leader sequence.

14. The nucleic acid sequence of any one of claims 1-13, wherein the nucleic acid sequence further comprises a promoter sequence.

15. The nucleic acid sequence of any one of claims 1-14, wherein the nucleic acid sequence is at least 80%, at least 90%, at least 95%, at least 99%, or 100% identical to a nucleic acid sequence set forth as SEQ ID NO: 1.

16. The nucleic acid sequence of any one of claims 1-15, wherein the first Type I CRISPR-Cas system is a Type I-A system, Type I-B system, Type I-C system, Type I-D system, Type I-E system, or Type I-F system.

17. The nucleic acid sequence of any one of claims 1-16, wherein the first Type I CRISPR-Cas system is a Type I-E system.

18. The nucleic acid sequence of any one of claims 1-17, wherein the second Type I CRISPR-Cas system is a Type I-A system, Type I-B system, Type I-C system, Type I-D system, Type I-E system, or Type I-F system.

19. The nucleic acid sequence of any one of claims 1-18, wherein the second Type I CRISPR-Cas system is a Type I-F system.

20. The nucleic acid sequence of any one of claims 1-19, wherein the first Type I CRISPR-Cas system, the second Type I CRISPR-Cas system, or both are endogenous to the target bacterium.

21. The nucleic acid sequence of any one of claims 1-19, wherein the first Type I CRISPR-Cas system, the second Type I CRISPR-Cas system, or both are exogenous to the target bacterium.

22. The nucleic acid sequence of any one of claims 1-20, wherein the target bacterium is E. coli.

23. The nucleic acid sequence of claim 22, wherein the E. coli is a multidrug-resistant (MDR) strain.

24. The nucleic acid sequence of claim 22, wherein the E. coli is an extended spectrum beta-lactamase (ESBL) strain.

25. The nucleic acid sequence of claim 22, wherein the E. coli is a carbapenem-resistant strain.

26. The nucleic acid sequence of claim 22, wherein the E. coli is a non-multidrug-resistant (non-MDR) strain.

27. The nucleic acid sequence of claim 22, wherein the E. coli is a non-carbapenem-resistant strain.

28. The nucleic acid sequence of any one of claims 20-25, wherein the E. coli causes urinary tract infection.

29. The nucleic acid sequence of any one of claims 22-28, wherein the E. coli causes inflammatory bowel disease (IBD).

30. A bacteriophage comprising a nucleic acid sequence comprising (a) a first CRISPR array designed to be operable with a first Type I CRISPR-Cas system, and (b) a second CRISPR array designed to be operable with a second Type I CRISPR-Cas system, wherein the first Type I CRISPR-Cas system and the second Type I CRISPR-Cas system are different Type I CRISPR-Cas systems.

31. The bacteriophage of claim 30, wherein the first CRISPR array comprises a first spacer sequence and the second CRISPR array comprises a second spacer sequence.

32. The bacteriophage of any one of claims 30-31, wherein the first CRISPR array and the second CRISPR array further comprises at least one repeat sequence.

33. The bacteriophage of claim 32, wherein the at least one repeat sequence is operably linked to the first spacer sequence at either its 5′ end or its 3′ end and/or the second spacer sequence at either its 5′ end or its 3′ end.

34. The bacteriophage of any one of claims 31-33, wherein the first spacer sequence and/or the second spacer sequence is complementary to a target nucleotide sequence of an essential gene in a target bacterium.

35. The bacteriophage of claim 34, wherein the target nucleotide sequence comprises all or a part of a promoter sequence of the essential gene.

36. The bacteriophage of claim 34, wherein the target nucleotide sequence comprises all or a part of a nucleotide sequence located on a coding strand of a transcribed region of the essential gene.

37. The bacteriophage of any one of claims 34-36, wherein the essential gene is ftsA.

38. The bacteriophage of any one of claims 30-36, wherein the first CRISPR array and the second CRISPR array are on same nucleic acid sequence.

39. The bacteriophage of any one of claims 30-38, wherein the nucleic acid sequence further comprises a leuO coding sequence.

40. The bacteriophage of any one of claims 30-39, wherein the nucleic acid sequence further comprises a leader sequence.

41. The bacteriophage of any one of claims 30-40, wherein the nucleic acid sequence further comprises a promoter sequence.

42. The bacteriophage of any one of claims 30-41, wherein the nucleic acid sequence is at least 80%, at least 90%, at least 95%, at least 99%, or 100% identical to a nucleic acid sequence set forth as SEQ ID NO: 1.

43. The bacteriophage of any one of claims 30-42, wherein the first Type I CRISPR-Cas system is a Type I-A system, Type I-B system, Type I-C system, Type I-D system, Type I-E system, or Type I-F system.

44. The bacteriophage of any one of claims 30-43, wherein the first Type I CRISPR-Cas system is a Type I-E system.

45. The bacteriophage of any one of claims 30-44, wherein the second Type I CRISPR-Cas system is a Type I-A system, Type I-B system, Type I-C system, Type I-D system, Type I-E system, or Type I-F system.

46. The bacteriophage of any one of claims 30-45, wherein the second Type I CRISPR-Cas system is a Type I-F system.

47. The bacteriophage of any one of claims 30-46, wherein the first Type I CRISPR-Cas system, the second Type I CRISPR-Cas system, or both are endogenous to the target bacterium.

48. The bacteriophage of any one of claims 30-46, wherein the first Type I CRISPR-Cas system, the second Type I CRISPR-Cas system, or both are exogenous to the target bacterium.

49. The bacteriophage of any one of claims 30-48, wherein the target bacterium is killed solely by lytic activity of the bacteriophage.

50. The bacteriophage of any one of claims 30-48, wherein the target bacterium is killed solely by activity of the first Type I CRISPR-Cas system or the second Type I CRISPR-Cas system.

51. The bacteriophage of any one of claims 30-48, wherein the target bacterium is killed by lytic activity of the bacteriophage in combination with activity of the first Type I CRISPR-Cas system or the second Type I CRISPR-Cas system.

52. The bacteriophage of any one of claims 30-48, wherein the target bacterium is killed by the activity of the first Type I CRISPR-Cas system or the second Type I CRISPR-Cas system, independently of the lytic activity of the bacteriophage.

53. The bacteriophage of any one of claims 30-48, wherein the activity of the first Type I CRISPR-Cas system or the second Type I CRISPR-Cas system supplements or enhances the lytic activity of the bacteriophage.

54. The bacteriophage of any one of claims 30-48, wherein the lytic activity of the bacteriophage and the activity of the first Type I CRISPR-Cas system or the second Type I CRISPR-Cas system are synergistic.

55. The bacteriophage of any one of claims 30-54, wherein the lytic activity of the bacteriophage, the activity of the first Type I CRISPR-Cas system or the second Type I CRISPR-Cas system, or both, is modulated by a concentration of the bacteriophage.

56. The bacteriophage of any one of claims 30-55, wherein the target bacterium is E. coli.

57. The bacteriophage of claim 56, wherein the E. coli is a multidrug-resistant (MDR) strain.

58. The bacteriophage of claim 56, wherein the E. coli is an extended spectrum beta-lactamase (ESBL) strain.

59. The bacteriophage of claim 56, wherein the E. coli is a carbapenem-resistant strain.

60. The bacteriophage of claim 56, wherein the E. coli is a non-multidrug-resistant (non-MDR) strain.

61. The bacteriophage of claim 56, wherein the E. coli is a non-carbapenem-resistant strain.

62. The bacteriophage of any one of claims 56-61, wherein the E. coli causes urinary tract infection.

63. The bacteriophage of any one of claims 56-62, wherein the E. coli causes inflammatory bowel disease (IBD).

64. The bacteriophage of any one of claims 30-63, wherein the bacteriophage is an obligate lytic bacteriophage.

65. The bacteriophage of any one of claims 30-63, wherein the bacteriophage is a temperate bacteriophage with a lysogeny gene removed, replaced, or inactivated, thereby rendering the bacteriophage lytic.

66. The bacteriophage of any one of claims 30-65, wherein the bacteriophage is PTA-126317, PTA-126320, PTA-126316, PTA-126324, PTA-126315, or PTA-126319.

67. The bacteriophage of any one of claims 30-66, wherein the nucleic acid sequence is inserted into a non-essential bacteriophage gene.

68. The bacteriophage of any one of claims 30-67, wherein the bacteriophage is at least 80%, at least 90%, at least 95%, at least 99%, or 100% identical to a bacteriophage deposited under ATCC Patent Deposit number PTA-126317.

69. The bacteriophage of any one of claims 30-68, wherein the bacteriophage is at least 80%, at least 90%, at least 95%, at least 99%, or 100% identical to a bacteriophage deposited under ATCC Patent Deposit number PTA-126320.

70. The bacteriophage of any one of claims 30-68, wherein the bacteriophage is at least 80%, at least 90%, at least 95%, at least 99%, or 100% identical to a bacteriophage deposited under ATCC Patent Deposit number PTA-126316.

71. The bacteriophage of any one of claims 30-68, wherein the bacteriophage is at least 80%, at least 90%, at least 95%, at least 99%, or 100% identical to a bacteriophage deposited under ATCC Patent Deposit number PTA-126324.

72. The bacteriophage of any one of claims 30-68, the bacteriophage is at least 80%, at least 90%, at least 95%, at least 99%, or 100% identical to a bacteriophage deposited under ATCC Patent Deposit number PTA-126315.

73. The bacteriophage of any one of claims 30-68, wherein the bacteriophage is at least 80%, at least 90%, at least 95%, at least 99%, or 100% identical to a bacteriophage deposited under ATCC Patent Deposit number PTA-126319.

74. A PTA-126317 bacteriophage comprising a nucleic acid sequence comprising (a) a first CRISPR array designed to be operable with a first Type I CRISPR-Cas system, or (b) a leuO coding sequence.

75. A PTA-126320 bacteriophage comprising a nucleic acid sequence comprising (a) a first CRISPR array designed to be operable with a first Type I CRISPR-Cas system, or (b) a leuO coding sequence.

76. A PTA-126316 bacteriophage comprising a nucleic acid sequence comprising (a) a first CRISPR array designed to be operable with a first Type I CRISPR-Cas system, or (b) a leuO coding sequence.

77. A PTA-126324 bacteriophage comprising a nucleic acid sequence comprising (a) a first CRISPR array designed to be operable with a first Type I CRISPR-Cas system, or (b) a leuO coding sequence.

78. A PTA-126315 bacteriophage comprising a nucleic acid sequence comprising (a) a first CRISPR array designed to be operable with a first Type I CRISPR-Cas system, or (b) a leuO coding sequence.

79. A PTA-126319 bacteriophage comprising a nucleic acid sequence comprising (a) a first CRISPR array designed to be operable with a first Type I CRISPR-Cas system, or (b) a leuO coding sequence.

80. The bacteriophage of any one of claims 74-79, wherein the nucleic acid sequence comprises (a) a first CRISPR array designed to be operable with a first Type I CRISPR-Cas system.

81. The bacteriophage of any one of claims 74-78, wherein the nucleic acid sequence comprises (b) a leuO coding sequence.

82. The bacteriophage of any one of claims 74-81, wherein the nucleic acid sequence comprises (a) a first CRISPR array designed to be operable with a first Type I CRISPR-Cas system, and (b) a leuO coding sequence.

83. The bacteriophage of any one of claims 74-82, wherein the nucleic acid sequence further comprises (c) a second CRISPR array designed to be operable with a second Type I CRISPR-Cas system.

84. The bacteriophage of claim 83, wherein the first Type I CRISPR-Cas system and the second Type I CRISPR-Cas system are different Type I CRISPR-Cas systems.

85. The bacteriophage of any one of claims 83-84, wherein the first CRISPR array comprises first spacer sequence and the second CRISPR array comprises a second spacer sequence.

86. The bacteriophage of any one of claims 83-85, wherein the first CRISPR array and the second CRISPR array further comprises at least one repeat sequence.

87. The bacteriophage of claim 86, wherein the at least one repeat sequence is operably linked to the first spacer sequence at either its 5′ end or its 3′ end and/or the second spacer sequence at either its 5′ end or its 3′ end.

88. The bacteriophage of any one of claims 85-86, wherein the first spacer sequence and/or the second spacer sequence is complementary to a target nucleotide sequence of an essential gene in a target bacterium.

89. The bacteriophage of claim 88, wherein the target nucleotide sequence comprises all or a part of a promoter sequence of the essential gene.

90. The bacteriophage of claim 88, wherein the target nucleotide sequence comprises all or a part of a nucleotide sequence located on a coding strand of a transcribed region of the essential gene.

91. The bacteriophage of any one of claims 88-90, wherein the essential gene is ftsA.

92. The bacteriophage of any one of claims 83-91, wherein the first CRISPR array and the second CRISPR array are on same nucleic acid sequence.

93. The bacteriophage of any one of claims 74-92, wherein the nucleic acid sequence further comprises a leader sequence.

94. The bacteriophage of any one of claims 74-93, wherein the nucleic acid sequence further comprises a promoter sequence.

95. The bacteriophage of any one of claims 74-94, wherein the nucleic acid sequence is at least 80%, at least 90%, at least 95%, at least 99%, or 100% identical to a nucleic acid sequence set forth as SEQ ID NO: 1.

96. The bacteriophage of any one of claims 74-95, wherein the first Type I CRISPR-Cas system is a Type I-A system, Type I-B system, Type I-C system, Type I-D system, Type I-E system, or Type I-F system.

97. The bacteriophage of any one of claims 74-96, wherein the first Type I CRISPR-Cas system is a Type I-E system.

98. The bacteriophage of any one of claims 83-97, wherein the second Type I CRISPR-Cas system is a Type I-A system, Type I-B system, Type I-C system, Type I-D system, Type I-E system, or Type I-F system.

99. The bacteriophage of any one of claims 83-97, wherein the second Type I CRISPR-Cas system is a Type I-F system.

100. The bacteriophage of any one of claims 88-99, wherein the first Type I CRISPR-Cas system, the second Type I CRISPR-Cas system, or both are endogenous to the target bacterium.

101. The bacteriophage of any one of claims 88-100, wherein the first Type I CRISPR-Cas system, the second Type I CRISPR-Cas system, or both are exogenous to the target bacterium.

102. The bacteriophage of any one of claims 88-101, wherein the target bacterium is killed solely by lytic activity of the bacteriophage.

103. The bacteriophage of any one of claims 88-102, wherein the target bacterium is killed solely by activity of the first Type I CRISPR-Cas system or the second Type I CRISPR-Cas system.

104. The bacteriophage of any one of claims 88-103, wherein the target bacterium is killed by lytic activity of the bacteriophage in combination with activity of the first Type I CRISPR-Cas system or the second Type I CRISPR-Cas system.

105. The bacteriophage of any one of claims 88-104, wherein the target bacterium is killed by the activity of the first Type I CRISPR-Cas system or the second Type I CRISPR-Cas system, independently of the lytic activity of the bacteriophage.

106. The bacteriophage of any one of claims 74-105, wherein the activity of the first Type I CRISPR-Cas system or the second Type I CRISPR-Cas system supplements or enhances the lytic activity of the bacteriophage.

107. The bacteriophage of any one of claims 74-106, wherein the lytic activity of the bacteriophage and the activity of the first Type I CRISPR-Cas system or the second Type I CRISPR-Cas system are synergistic.

108. The bacteriophage of any one of claims 74-107, wherein the lytic activity of the bacteriophage, the activity of the first Type I CRISPR-Cas system or the second Type I CRISPR-Cas system, or both, is modulated by a concentration of the bacteriophage.

109. The bacteriophage of any one of claims 88-108, wherein the target bacterium is E. coli.

110. The bacteriophage of claim 109, wherein the E. coli is a multidrug-resistant (MDR) strain.

111. The bacteriophage of claim 109, wherein the E. coli is an extended spectrum beta-lactamase (ESBL) strain.

112. The bacteriophage of claim 109, wherein the E. coli is a carbapenem-resistant strain.

113. The bacteriophage of claim 109, wherein the E. coli is a non-multidrug-resistant (non-MDR) strain.

114. The bacteriophage of claim 109, wherein the E. coli is a non-carbapenem-resistant strain.

115. The bacteriophage of any one of claims 109-114, wherein the E. coli causes urinary tract infection.

116. The bacteriophage of any one of claims 109-115, wherein the E. coli causes inflammatory bowel disease (IBD).

117. The bacteriophage of any one of claims 74-116, wherein the bacteriophage is an obligate lytic bacteriophage.

118. The bacteriophage of any one of claims 74-117, wherein the nucleic acid sequence is inserted into a non-essential bacteriophage gene.

119. The bacteriophage of any one of claim 74 or 80-118, wherein the bacteriophage is at least 80%, at least 90%, at least 95%, at least 99%, or 100% identical to a bacteriophage deposited under ATCC Patent Deposit number PTA-126317.

120. The bacteriophage of any one of claim 75, or 80-118, wherein the bacteriophage is at least 80%, at least 90%, at least 95%, at least 99%, or 100% identical to a bacteriophage deposited under ATCC Patent Deposit number PTA-126320.

121. The bacteriophage of any one of claim 76, or 80-118, wherein the bacteriophage is at least 80%, at least 90%, at least 95%, at least 99%, or 100% identical to a bacteriophage deposited under ATCC Patent Deposit number PTA-126316.

122. The bacteriophage of any one of claim 77, or 80-118, wherein the bacteriophage is at least 80%, at least 90%, at least 95%, at least 99%, or 100% identical to a bacteriophage deposited under ATCC Patent Deposit number PTA-126324.

123. The bacteriophage of any one of claim 78, or 80-118, wherein the bacteriophage is at least 80%, at least 90%, at least 95%, at least 99%, or 100% identical to a bacteriophage deposited under ATCC Patent Deposit number PTA-126315.

124. The bacteriophage of any one of claims 79-118, wherein the bacteriophage is at least 80%, at least 90%, at least 95%, at least 99%, or 100% identical to a bacteriophage deposited under ATCC Patent Deposit number PTA-126319.

125. A composition comprising:
at least two bacteriophages selected from a list consisting of:
(i) a bacteriophage comprising at least 80% identity to a bacteriophage deposited under ATCC Patent Deposit number PTA-126317,
(ii) a bacteriophage comprising at least 80% identity to a bacteriophage deposited under ATCC Patent Deposit number PTA-126320,
(iii) a bacteriophage comprising at least 80% identity to a bacteriophage deposited under ATCC Patent Deposit number PTA-126316,
(iv) a bacteriophage comprising at least 80% identity to a bacteriophage deposited under ATCC Patent Deposit number PTA-126324,
(v) a bacteriophage comprising at least 80% identity to a bacteriophage deposited under ATCC Patent Deposit number PTA-126315, and
(vi) a bacteriophage comprising at least 80% identity to a bacteriophage deposited under ATCC Patent Deposit number PTA-126319.

126. A composition comprising:
at least three bacteriophages selected from a list consisting of:
(i) a bacteriophage comprising at least 80% identity to a bacteriophage deposited under ATCC Patent Deposit number PTA-126317,
(ii) a bacteriophage comprising at least 80% identity to a bacteriophage deposited under ATCC Patent Deposit number PTA-126320,
(iii) a bacteriophage comprising at least 80% identity to a bacteriophage deposited under ATCC Patent Deposit number PTA-126316,
(iv) a bacteriophage comprising at least 80% identity to a bacteriophage deposited under ATCC Patent Deposit number PTA-126324,
(v) a bacteriophage comprising at least 80% identity to a bacteriophage deposited under ATCC Patent Deposit number PTA-126315, and
(vi) a bacteriophage comprising at least 80% identity to a bacteriophage deposited under ATCC Patent Deposit number PTA-126319.

127. A composition comprising:
at least six bacteriophages selected from a list consisting of:
(i) a bacteriophage comprising at least 80% identity to a bacteriophage deposited under ATCC Patent Deposit number PTA-126317,
(ii) a bacteriophage comprising at least 80% identity to a bacteriophage deposited under ATCC Patent Deposit number PTA-126320,
(iii) a bacteriophage comprising at least 80% identity to a bacteriophage deposited under ATCC Patent Deposit number PTA-126316,
(iv) a bacteriophage comprising at least 80% identity to a bacteriophage deposited under ATCC Patent Deposit number PTA-126324,
(v) a bacteriophage comprising at least 80% identity to a bacteriophage deposited under ATCC Patent Deposit number PTA-126315, and
(vi) a bacteriophage comprising at least 80% identity to a bacteriophage deposited under ATCC Patent Deposit number PTA-126319.

128. A composition comprising:
(a) a bacteriophage comprising at least 80% identity to a bacteriophage deposited under ATCC Patent Deposit number PTA-126324;
(b) a bacteriophage comprising at least 80% identity to a bacteriophage deposited under ATCC Patent Deposit number PTA-126315; and
(c) a bacteriophage comprising at least 80% identity to a bacteriophage deposited under ATCC Patent Deposit number PTA-126319.

129. The composition of any one of claims 125-128, wherein (a) the bacteriophage comprises at least 90%, at least 95%, at least 99%, or 100% identity to a bacteriophage deposited under ATCC Patent Deposit number PTA-126324.

130. The composition of any one of claims 125-129, wherein (b) the bacteriophage comprises at least 90%, at least 95%, at least 99%, or 100% identity to a bacteriophage deposited under ATCC Patent Deposit number PTA-126315.

131. The composition of any one of claims 125-130, wherein (c) the bacteriophage comprises at least 90%, at least 95%, at least 99%, or 100% identity to a bacteriophage deposited under ATCC Patent Deposit number PTA-126319.

132. A composition comprising:
(a) a bacteriophage comprising at least 80% identity to a bacteriophage deposited under ATCC Patent Deposit number PTA-126317, and
(b) at least one more bacteriophage selected from the list consisting of:
(i) a bacteriophage comprising at least 80% identity to a bacteriophage deposited under ATCC Patent Deposit number PTA-126320,
(ii) a bacteriophage comprising at least 80% identity to a bacteriophage deposited under ATCC Patent Deposit number PTA-126316,
(iii) a bacteriophage comprising at least 80% identity to a bacteriophage deposited under ATCC Patent Deposit number PTA-126324,
(iv) a bacteriophage comprising at least 80% identity to a bacteriophage deposited under ATCC Patent Deposit number PTA-126315, and
(v) a bacteriophage comprising at least 80% identity to a bacteriophage deposited under ATCC Patent Deposit number PTA-126319.

133. A composition comprising:
(a) a bacteriophage comprising at least 80% identity to a bacteriophage deposited under ATCC Patent Deposit number PTA-126320, and
(b) at least one more bacteriophage selected from the list consisting of:
(i) a bacteriophage comprising at least 80% identity to a bacteriophage deposited under ATCC Patent Deposit number PTA-126317,
(ii) a bacteriophage comprising at least 80% identity to a bacteriophage deposited under ATCC Patent Deposit number PTA-126316,
(iii) a bacteriophage comprising at least 80% identity to a bacteriophage deposited under ATCC Patent Deposit number PTA-126324,
(iv) a bacteriophage comprising at least 80% identity to a bacteriophage deposited under ATCC Patent Deposit number PTA-126315, and
(v) a bacteriophage comprising at least 80% identity to a bacteriophage deposited under ATCC Patent Deposit number PTA-126319.

134. A composition comprising:
(a) a bacteriophage comprising at least 80% identity to a bacteriophage deposited under ATCC Patent Deposit number PTA-126316, and
(b) at least one more bacteriophage selected from the list consisting of:
(i) a bacteriophage comprising at least 80% identity to a bacteriophage deposited under ATCC Patent Deposit number PTA-126317,
(ii) a bacteriophage comprising at least 80% identity to a bacteriophage deposited under ATCC Patent Deposit number PTA-126320,
(iii) a bacteriophage comprising at least 80% identity to a bacteriophage deposited under ATCC Patent Deposit number PTA-126324,
(iv) a bacteriophage comprising at least 80% identity to a bacteriophage deposited under ATCC Patent Deposit number PTA-126315, and
(v) a bacteriophage comprising at least 80% identity to a bacteriophage deposited under ATCC Patent Deposit number PTA-126319.

135. A composition comprising:
(a) a bacteriophage comprising at least 80% identity to a bacteriophage deposited under ATCC Patent Deposit number PTA-126324, and
(b) at least one more bacteriophage selected from the list consisting of:
(i) a bacteriophage comprising at least 80% identity to a bacteriophage deposited under ATCC Patent Deposit number PTA-126317,
(ii) a bacteriophage comprising at least 80% identity to a bacteriophage deposited under ATCC Patent Deposit number PTA-126320,
(iii) a bacteriophage comprising at least 80% identity to a bacteriophage deposited under ATCC Patent Deposit number PTA-126316,
(iv) a bacteriophage comprising at least 80% identity to a bacteriophage deposited under ATCC Patent Deposit number PTA-126315, and
(v) a bacteriophage comprising at least 80% identity to a bacteriophage deposited under ATCC Patent Deposit number PTA-126319.

136. A composition comprising:
(a) a bacteriophage comprising at least 80% identity to a bacteriophage deposited under ATCC Patent Deposit number PTA-126315, and
(b) at least one more bacteriophage selected from the list consisting of:
(i) a bacteriophage comprising at least 80% identity to a bacteriophage deposited under ATCC Patent Deposit number PTA-126317,
(ii) a bacteriophage comprising at least 80% identity to a bacteriophage deposited under ATCC Patent Deposit number PTA-126320,
(iii) a bacteriophage comprising at least 80% identity to a bacteriophage deposited under ATCC Patent Deposit number PTA-126316,
(iv) a bacteriophage comprising at least 80% identity to a bacteriophage deposited under ATCC Patent Deposit number PTA-126324,
(v) a bacteriophage comprising at least 80% identity to a bacteriophage deposited under ATCC Patent Deposit number PTA-126319.

137. A composition comprising:
(a) a bacteriophage comprising at least 80% identity to a bacteriophage deposited under ATCC Patent Deposit number PTA-126319, and
(b) at least one more bacteriophage selected from the list consisting of:
(i) a bacteriophage comprising at least 80% identity to a bacteriophage deposited under ATCC Patent Deposit number PTA-126317,
(ii) a bacteriophage comprising at least 80% identity to a bacteriophage deposited under ATCC Patent Deposit number PTA-126320,
(iii) a bacteriophage comprising at least 80% identity to a bacteriophage deposited under ATCC Patent Deposit number PTA-126316,
(iv) a bacteriophage comprising at least 80% identity to a bacteriophage deposited under ATCC Patent Deposit number PTA-126324, and
(v) a bacteriophage comprising at least 80% identity to a bacteriophage deposited under ATCC Patent Deposit number PTA-126315.

138. A pharmaceutical composition comprising:
(a) (i) the nucleic acid sequence of any one of claims 1-29, (ii) the bacteriophage of any one of claims 30-124, or (iii) the composition of any one of claims 126-137; and
(b) a pharmaceutically acceptable excipient.

139. The pharmaceutical composition of claim 138, wherein the pharmaceutical composition is in the form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof.

140. A method of killing a target bacterium comprising introducing into a target bacterium (a) the bacteriophage of any one of claims 30-124, (b) the composition of any one of claims 126-137, or (c) the pharmaceutical composition of any one of claims 138-139.

141. A method modifying a mixed population of bacterial cells having a first bacterial species that comprises a target nucleotide sequence in the essential gene and a second bacterial species that does not comprise a target nucleotide sequence in the essential gene, the method comprising introducing into the mixed population of bacterial cells (a) the bacteriophage of any one of claims 30-124, (b) the composition of any one of claims 126-137, or (c) the pharmaceutical composition of any one of claims 138-139.

142. A method of treating a disease in an individual in need thereof, the method comprising administering to the individual ((a) the bacteriophage of any one of claims 30-124, (b) the composition of any one of claims 126-137, or (c) the pharmaceutical composition of any one of claims 138-139.

143. The method of claim 142, wherein the disease is a bacterial infection.

144. The method of claim 142, wherein the disease is a urinary tract infection (UTI).

145. The method of claim 142, wherein the disease is inflammatory bowel disease (IBD).

146. The method of any one of claims 142-145, wherein the individual is a mammal.

147. The method of any one of claims 142-146, wherein the administering is intra-arterial, intravenous, intraurethral, intramuscular, oral, subcutaneous, inhalation, or any combination thereof.

148. The method of any one of claims 142-147, wherein (a) the bacteriophage, (b) the composition, or (c) the pharmaceutical composition is administered at a dose of phage between 10⁶ and 10¹⁰ PFU.

149. The method of any one of claims 142-148, wherein (a) the bacteriophage, (b) the composition, or (c) the pharmaceutical composition is administered 1, 2, 3, 4, or 5 times daily.

150. The method of any one of claims 142-149, wherein (a) the bacteriophage, (b) the composition, or (c) the pharmaceutical composition is administered 2 times daily.

151. The method of any one of claims 143-150, wherein (a) the bacteriophage, (b) the composition, or (c) the pharmaceutical composition is administered every 12 hours.

152. A method of treating a urinary tract infection (UTI) in an individual in need thereof, the method comprising administering to the individual (a) the bacteriophage of any one of claims 30-124, (b) the composition of any one of claims 126-137, or (c) the pharmaceutical composition of any one of claims 138-139.

153. The method of claim 152, wherein the UTI is caused by E. coli.

154. The method of claim 153, wherein the E. coli is a multidrug-resistant (MDR) strain.

155. The method of claim 153, wherein the E. coli is an extended spectrum beta-lactamase (ESBL) strain.

156. The method of claim 153, wherein the E. coli is a carbapenem-resistant strain.

157. The method of claim 153, wherein the E. coli is a non-multidrug-resistant (non-MDR) strain.

158. The method of claim 153, wherein the E. coli is a non-carbapenem-resistant strain.

159. The method of any one of claims 152-158, wherein the individual is a mammal.

160. The method of any one of claims 152-159, wherein the administering is intra-arterial, intravenous, intraurethral, intramuscular, oral, subcutaneous, inhalation, or any combination thereof.

161. The method of any one of claims 152-160, wherein (a) the bacteriophage, (b) the composition, or (c) the pharmaceutical composition is administered at a dose of phage between 10⁶ and 10¹⁰ PFU.

162. The method of any one of claims 152-161, wherein (a) the bacteriophage, (b) the composition, or (c) the pharmaceutical composition is administered 1, 2, 3, 4, or 5 times daily.

163. The method of any one of claims 152-162, wherein (a) the bacteriophage, (b) the composition, or (c) the pharmaceutical composition is administered 2 times daily.

164. The method of any one of claims 152-163, wherein (a) the bacteriophage, (b) the composition, or (c) the pharmaceutical composition is administered every 12 hours.


SEQUENCE LISTING (1 sequence)

SEQ ID NO: 1 - DNA, 1362 bp, Unknown
  Description of Unknown bacteriophage sequence
gataattagtgctgcgggtaggtaaggataaaaaagggtggcagcaggagattgagatggttttgctttattaacaacgggctaaacgtgtagtatttgagctagcgaattcgagctcggtaccacctcgagttccccgcgccagcggggataaaccgctgaagtagaaaaacgtgttacagcatcagtcgagttccccgcgccagcggggataaaccgggagagagtgagcgatcctccgttaacatagttcactgccgtacaggcagcttagaaagtattattcgacggcggtgggattgcttcacgttcactgccgtacaggcagcttagaaaatcaaaattgctgtctgccaggtgatcgctttgacagctagctcagtcctaggtactgtgctagcataaaggaggtaaataatgccagaggtacaaacagatcatccagagacagcggagttaagcaaaccacagctacgcatggtcgatctcaacttattaaccgttttcgatgccgtgatgcaggagcaaaacattactcgtgccgctcatgttctgggaatgtcgcaacctgcggtcagtaacgctgttgcacgcctgaaggtgatgtttaatgacgagctttttgttcgttatggccgtggtattcaaccgactgctcgcgcatttcaactttttggttcagttcgtcaggcattgcaactagtacaaaatgaattgcctggttcaggttttgaacccgcgagcagtgaacgtgtatttcatctttgtgtttgcagcccgttagacagcattctgacctcgcagatttataatcacattgagcagattgcgccaaatatacatgttatgttcaagtcgtcattaaatcagaacactgaacatcagctgcgttatcaggaaacggagtttgtgattagttatgaggacttccatcgtcctgaatttaccagcgtaccattatttaaagatgaaatggtgctggtagccagcaaaaatcatccaacaattaagggcccgttactgaaacatgatgtttataacgaacaacatgcggcggtttcgctcgatcgtttcgcgtcatttagtcaaccttggtatgacacggtagataagcaagccagtatcgcgtatcagggcatggcaatgatgagcgtacttagcgtggtgtcgcaaacgcatttggtcgctattgcgccgcgttggctggctgaagagttcgctgaatccttagaattacaggtattaccgctgccgttaaaacaaaacagcagaacctgttatctctcctggcatgaagctgccgggcgcgataaaggccatcagtggatggaagagcaattagtctcaatttgcaaacgctaa